(19) United States
(12) Patent Application Publication
(10) Pub. No.: US 2023/0181644 A1
(43) Pub. Date: Jun. 15, 2023

(54) METHODS OF GENERATING CELLS

(71) Applicant: LYELL IMMUNOPHARMA, INC., South San Francisco, CA (US)

(72) Inventor: Suman Kumar VODNALA, San Mateo, CA (US)

(73) Assignee: LYELL IMMUNOPHARMA, INC., South San Francisco, CA (US)

(21) Appl. No.: 18/050,417

(22) Filed: Oct. 27, 2022

Related U.S. Application Data
(60) Provisional application No. 63/273,137, filed on Oct. 28, 2021.

(51) Int. Cl. A61K 35/17; C07K 14/725; C12N 15/67; C12N 5/078

(52) U.S. Cl. CPC A61K 35/17; C07K 14/7051; C12N 15/67; C12N 5/0634; C07K 2319/02; C07K 2319/03; C07K 2319/33

(57) ABSTRACT

The present disclosure provides methods of preparing immune cells, e.g., T cells and/or NK cells, comprising contacting the cells with programmable cell-signaling scaffolds in a medium comprising at least about 5 mM potassium ion. In some aspects, the methods disclosed herein increase the number of less-differentiated cells in the population of cells. In some aspects, the cultured cells are engineered, e.g., to comprise a chimeric antigen receptor (CAR) or an engineered T cell receptor (TCR). In some aspects, the cells are administered to a subject in need thereof.

### CROSS-REFERENCE TO RELATED APPLICATIONS

This application claims priority benefit of U.S. Provisional Application No. 63/273,137, filed Oct. 28, 2021, which is herein incorporated by reference in its entirety.

### FIELD

The present disclosure relates to methods of culturing cells, e.g., pluripotent, multipotent, and/or immune cells (e.g., T cells, NK cells, and/or TILs). In some aspects, the methods disclosed herein promote enrichment of less-differentiated cells and/or undifferentiated cells in culture. Cells cultured using the methods disclosed herein can be used for various cell therapies, including but not limited to chimeric antigen receptor (CAR) T cell therapy and TCR T cell therapy including neoantigen directed-T cell therapies.

### BACKGROUND

Cancer immunotherapy relies on harnessing T cells—the immune system's primary killers of infected and diseased cells—to attack and kill tumor cells. However, there is an important stumbling block for immunotherapy: T cells' ability to kill can fade, a phenomenon often referred to as exhaustion or terminal differentiation of T cells. Immune checkpoint blockade, ex vivo-expanded Tumor-Infiltrating Lymphocytes (TILs) therapy, chimeric antigen receptor (CAR) T cell therapy, and T cell receptor-engineered (TCR) T cell therapy are treatments that make use of functionally active T cells isolated from patients and require highly functional T cells in order to be effective. These T cells are engineered and expanded ex vivo to recognize antigens on target cancer cells. T cell therapies have not been consistently effective at curing solid cancers, in part because the T cells lose their ability to proliferate or kill over time.

One means of overcoming T cell exhaustion is to selectively administer T cells having a less-differentiated state. For example, T memory stem cells (T_{SCM}) persist for a greater period in patients following administration than do more differentiated T central memory (T_{CM}) or T effector memory (T_{EM}) cells, and T_{SCM} elicit a more pronounced and prolonged effect on tumor size than more differentiated cells. However, there remains a need in the art for methods of efficiently enriching for less differentiated and/or naïve T cells from a mixed population of isolated T cells.

### BRIEF SUMMARY

Some aspects of the present disclosure are directed to methods of preparing a population of human immune cells for immunotherapy comprising contacting human immune cells with a programmable cell-signaling scaffold (PCS) in a medium comprising potassium ion at a concentration higher than 5 mM.

Some aspects of the present disclosure are directed to methods of activating a population of human immune cells for immunotherapy comprising contacting human immune cells with a programmable cell-signaling scaffold (PCS) in a medium comprising potassium ion at a concentration higher than 5 mM.

Some aspects of the present disclosure are directed to methods of increasing the yield of activated human immune cells during ex vivo or in vitro culture comprising contacting human immune cells with a programmable cell-signaling scaffold (PCS) in a medium comprising potassium ion at a concentration higher than 5 mM.

Some aspects of the present disclosure are directed to methods of increasing stemness of activated human immune cells while increasing the yield of activated human immune cells during ex vivo or in vitro culture for an immunotherapy comprising contacting human immune cells with a programmable cell-signaling scaffold (PCS) in a medium comprising potassium ion at a concentration higher than 5 mM.

Some aspects of the present disclosure are directed to methods of expanding a population of activated stem-like immune cells ex vivo or in vitro comprising contacting immune cells with a programmable cell-signaling scaffold (PCS) in a medium comprising potassium ion at a concentration higher than 5 mM.

In some aspects, the PCS comprises (i) a base layer comprising high surface area mesoporous silica micro-rods (MSR); (ii) a continuous, fluid-supported lipid bilayer (SLB) layered on the MSR base layer; (iii) a plurality of surface cues loaded onto the scaffold; and (iv) a plurality of soluble cues loaded onto the scaffold.

In some aspects, the surface cue is loaded onto the SLB layer. In some aspects, the soluble cue is loaded onto the MSR base layer.

In some aspects, the soluble cue is released from the scaffold in a controlled-release manner. In some aspects, the soluble cue is released from the scaffold in a sustained manner for at least 30 days.

In some aspects, the plurality of soluble cues comprises IL-1, IL-2, IL-4, IL-5, IL-7, IL-10, IL-12, IL-15, IL-17, IL-21, transforming growth factor beta (TGF-β), or an agonist thereof, a mimetic thereof, a variant thereof, a functional fragment thereof, or a combination thereof. In some aspects, the plurality of soluble cues comprises (i) IL-2, an agonist thereof, a mimetic thereof, a variant thereof, a functional fragment thereof, or a combination thereof and (ii) a second soluble cue comprising IL-7, IL-21, IL-15, IL-15 superagonist, or any combination thereof. In some aspects, the plurality of soluble cues comprises (i) IL-2, an agonist thereof, a mimetic thereof, a variant thereof, a functional fragment thereof, or a combination thereof, (ii) a second soluble cue comprising IL-7, IL-21, IL-15, IL-15 superagonist, or any combination thereof, and (iii) a third soluble cue comprising IL-7, IL-21, IL-15, IL-15 superagonist, or any combination thereof. In some aspects, the plurality of soluble cues comprises an N-terminal IL-2 fragment comprising the first 30 amino acids of IL-2 (pl-30), an IL-2 superkine peptide, an IL-2 partial agonist peptide, or a combination thereof.

In some aspects, the plurality of surface cues comprises a T-cell stimulatory molecule, a T-cell co-stimulatory molecule, or both a T-cell stimulatory molecule and a T cell co-stimulatory molecule. In some aspects, the T-cell stimulatory molecule and the T-cell co-stimulatory molecule are each, independently, loaded onto the fluid-supported lipid bilayer (SLB).

In some aspects, the T-cell stimulatory molecule and the T-cell co-stimulatory molecule are loaded via affinity pairing or chemical coupling. In some aspects, the affinity coupling comprises a biotin-streptavidin pair, an antibody-antigen pair, an antibody-hapten pair, an affinity pair, a capture protein pair, an Fc receptor-IgG pair, a metal-chelating lipid pair, or a combination thereof. In some aspects, the chemical coupling comprises azide-alkyne chemical (AAC) reaction, dibenzo-cyclooctyne ligation (DCL), tetrazine-alkene ligation (TAL), or any combination thereof.

In some aspects, the T-cell stimulatory molecule and the T-cell co-stimulatory molecule are each, independently, coated onto the fluid-supported lipid bilayer (SLB). In some aspects, the T-cell stimulatory molecule and the T-cell co-stimulatory molecule are each, independently, partly embedded onto the fluid-supported lipid bilayer (SLB). In some aspects, the T-cell stimulatory molecule and T-cell co-stimulatory molecule are each, independently, loaded onto the mesoporous silica micro-rods (MSR). In some aspects, the T-cell stimulatory molecule and the T-cell co-stimulatory molecule are each, independently, antibody molecules or antigen-binding fragments thereof.

In some aspects, the T-cell stimulatory molecule comprises an anti-CD3 antibody or an antigen-binding portion thereof, an anti-macrophage scavenger receptor (MSR1) antibody or an antigen-binding portion thereof, an anti-T-cell receptor (TCR) antibody or an antigen-binding portion thereof, an anti-CD2 antibody or an antigen-binding portion thereof, an anti-CD47 antibody or an antigen-binding portion thereof, a major histocompatibility complex (MIC) molecule loaded with an MHC peptide or a multimer thereof, an MHC-immunoglobulin (Ig) conjugate or a multimer thereof, or a combination thereof.

In some aspects, the T-cell co-stimulatory molecule comprises an antibody, or an antigen-binding portion thereof, which specifically binds to a co-stimulatory antigen comprising CD28, 4.1BB (CD137), OX40 (CD134), CD27 (TNFRSF7), GITR (CD357), CD30 (TNFRSF8), HVEM (CD270), LTfiR (TNFRSF3), DR3 (TNFRSF25), ICOS (CD278), CD226 (DNAM1), CRTAM (CD355), TIM1 (HAVCR1, KIM1), CD2 (LFA2, 0X34), SLAM (CD150, SLAMF1), 2B4 (CD244, SLAMF4), Ly108 (NTBA, CD352, SLAMF6), CD84 (SLAMF5), Ly9 (CD229, SLAMF3), CRACC (CD319, BLAME), or any combination thereof.

In some aspects, the T-cell stimulatory molecule and the T-cell co-stimulatory molecule comprise bispecific antibodies or antigen binding portions thereof. In some aspects, the T-cell stimulatory molecule and T-cell co-stimulatory molecule comprise a pair comprising CD3/CD28, CD3/ICOS, CD3/CD27, CD3/CD137, or a combination thereof.

In some aspects, the scaffold further comprises an immunoglobulin molecule that binds specifically to an Fc-fusion protein.

In some aspects, the scaffold further comprises a recruitment compound comprising granulocyte macrophage-colony stimulating factor (GM-CSF), chemokine (C—C motif) ligand 21 (CCL-21), chemokine (C—C motif) ligand 19 (CCL-19), Chemokine (C—X—C Motif) ligand 12 (CXCL12), interferon gamma (IFNy), a FMS-like tyrosine kinase 3 (Flt-3) ligand, or any combination thereof. In some aspects, the recruitment compound comprises granulocyte macrophage colony stimulating factor (GM-CSF).

In some aspects, the scaffold further comprises an antigen. In some aspects, the antigen comprises a tumor antigen. In some aspects, the tumor antigen is adenomatous polyposis coli protein (APC), adenosine deaminase-binding protein (AD Abp), a-fetoprotein, AFP (alpha-fetoprotein), AIM-2, AIM-3, and WT1), ART1, ART4, B7-H3, B7-H6, BAGE, BCMA, B-cyclin, BMI1, Braf, brain glycogen phosphorylase, BRAP, C13orf24, C6orf153, C9orf 112, CA-125, CA9 (carbonic anhydrase 9), CASP-8, cathepsin B, Cav-1, CCL-1 (C—C motif chemokine ligand 1), CD123, CD138, CD171, CD19, CD20, CD21, CD22, CD23, CD24, CD30, CD33, CD352, CD38, CD40, CD44, CD44v6, CD44v7/8, CD45, CD47, CD5, CD56, CD66e, CD70, CD74, CD74, CD79a, CD79b, CD98, cdc27, CDK-1, CDK4, CEA, CEA (carcinoembryonic antigen), c-erbB-2, Claudin 18.2, Claudin 6, c-MET, Colorectal associated antigen (CRC)-C017-1A/GA733, Connexin 37, COX-2, CT-7, cyclophilin b, CYNL2, Dipeptidyl peptidase IV (DPPIV), DLL3 (delta-like protein 3), DLL4, EBV-encoded nuclear antigen (EBNA)-I, E-cadherin, EGFRvIII, ENPP3 (ectonucleotide pyrophosphatase/phosphodiesterase family member 3), EpCAM, EPG-2 (epithelial glycoprotein 2), EPG-40, EPHa2 (ephrine receptor A2), EphA2/Eck, ephrinB2, ERBB dimers, ESO-1, estrogen receptor, ETBR (endothelin B receptor), EZH2, FAP-α (fibroblast activation protein α), FBP (a folate binding protein), FCRL5, fetal AchR (fetal acetylcholine receptor), fodrin, Fra-1/Fosl 1, FR-α (folate receptor alpha), GAGE-1, GAGE-family of tumor antigens, Ganglioside/GD2, GCC (guanyl cyclase C), GD2, GD2 gangliosides, GD3, GLEA2, GM2, GnT-V, GnT-V, GOLGA, gp100 (glycoprotein 100), gp75, GPC2 (glypican-2), GPC3, gplOO, GPNMB (glycoprotein NMB), GPRC5D (G Protein Coupled Receptor 5D), GUI, H60, hepatitis B surface antigen, HER2, HER3, HER4, HLA-A complexed with peptides derived from AFP, HLA-A1 (human leukocyte antigen A1), HLA-A2 (human leukocyte antigen A2), HMW-MAA (human high molecular weight-melanoma-associated antigen), HSPH1, Ig kappa, Ig lambda, IGF1R (insulin-like growth factor 1 receptor), Ig-idiotype, IL-13Ra2 (IL-13 receptor alpha 2), IL13Ralpha, IL-22Ra (IL-22 receptor alpha), ING4, KDR (kinase insert domain receptor), Ki67, KIAA0376, KRAS, Ku70/80, LAGE-I, Lewis Y, LI cell adhesion molecule (LI-CAM), Liv-1, Livin, lmp-1, LRRC8A (leucine rich repeat containing 8 Family member A), MAGE-1, MAGE-2, MAGE-3, MAGE-A, MAGE-A3, MAGE-A6, MART-1 (melan A), MCSP (melanoma-associated chondroitin sulfate proteoglycan), melanoma-associated antigen (MAGE)-A1, mesothelin, MHC/peptide complexes (e.g., MICA, MICB, midkin, MRP-3, MUC16, mucin 1 (MUC1), MUM-1, murine cytomegalovirus (MCMV), NAG, NCAM (neural cell adhesion molecule), Nectin-4, Nestin, NKG2D (natural killer group 2 member D) ligands, NKTR, NSEP1, NY-ESO, NY-ESO-1, OLIG2, oncofetal antigen, P1A, p53, PAP, PD-1, PD-L1, pl20ctn, p15, Pmell 17, PRAME (preferentially expressed antigen of melanoma), progesterone receptor, PROX1, PSA (prostate specific antigen), PSCA (prostate stem cell antigen), PSMA, PSMA (prostate specific membrane antigen), RAE-1 proteins, RAGE, ras, RBPSUH, RCAS1, ROR1, ROR2, RTN4, SART1, SART2, SART3, SCP-I, SIRPα (signal-regulatory protein alpha), SLIT, SLITRK6 (NTRK-like protein 6), Smad family of tumor antigens, SOX10, SOX11, SOX2, SSX-2 (HOM-MEL-40), SSX-4, SSX-5, SSX-I, SSX-I, STEAP1 (six transmembrane epithelial antigen of the prostate 1), Survivin, survivin, TAG72 (tumor-associated glycoprotein 72), T-cell receptor/CD3-zeta chain, TNKS2, TPBG (trophoblast glycoprotein), TPR, Trop-2, TRP-1, TRP-2, Tyrosinase, U2AF1L, UL16-binding protein-like transcript 1 (Mult1), UPAR, VEGFR1 (vascular endothelial growth factor receptor 1), VEGFR2, WT-1, αvβ6 or another integrin, β-catenin, β1,6-N, β-catenin, γ-catenin, íívíηβ, and antigens from HIV, HBV, HCV, HPV, and other pathogens, a patient-specific neoantigen, or an immunogenic peptide thereof, and any combination thereof.

In some aspects, the weight ratio of the supported lipid bilayer (SLB) to the mesoporous silica micro-rods (MSR) is between about 10:1 and about 1:20. In some aspects, the continuous, fluid-supported lipid bilayer (SLB) comprises a lipid selected from the group consisting of (DMPC), dipalmitoylphosphatidylcholine (DPPC), distearoylphosphatidylcholine (DSPC), palmitoyl-oleoylphosphatidylcholine (POPC), dioleoylphosphatidylcholine (DOPC), dioleoylphosphatidylethanolamine (DOPE), dimyristoylphosphatidylethanolamine (DMPE) and dipalmitoylphosphatidylethanolamine (DPPE), 1-stearoyl-2-myristoyl-sn-glycero-3-phosphocholine (8:0-14:0 PC), or a combination thereof. In some aspects, the mesoporous silica microrod-lipid bilayer (MSR-SLB) scaffold retains a continuous, fluid architecture for at least 14 days. In some aspects, the dry weight ratio of the mesoporous silica micro-rods (MSR) to the T-cell activating/co-stimulatory molecules is between 1:1 to 50:1.

In some aspects, the method further comprises modifying the immune cells with a polynucleotide encoding a ligand binding protein.

In some aspects, the immune cells comprise a polynucleotide encoding an antigen receptor. In some aspects, the antigen receptor is selected from an antibody, an engineered antibody such as scFv, a CAR, an engineered TCR, a TCR mimic, a chimeric signaling receptor (CSR), or any combination thereof. In some aspects, the CAR is designed as a standard CAR, a split CAR, an off-switch CAR, an on-switch CAR, a first-generation CAR, a second-generation CAR, a third-generation CAR, or a fourth-generation CAR.

In some aspects, the antigen receptor comprises (i) an antigen-binding domain, (ii) a transmembrane domain, (iii) a costimulatory domain, (iv) an intracellular signaling domain, or (v) any combination of (i)-(iv).

In some aspects, the antigen-binding domain specifically binds an antigen selected from the group consisting of AFP (alpha-fetoprotein), avP6 or another integrin, BCMA, Braf, B7-H3, B7-H6, CA9 (carbonic anhydrase 9), CCL-1 (C—C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof. In some aspects, the antigen-binding domain specifically binds ROR1. In some aspects, the antigen-binding domain specifically binds GPC2.

In some aspects, the costimulatory domain comprises a costimulatory domain of an interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, or any combination thereof. In some aspects, the costimulatory domain comprises a 4-1BB/CD137 costimulatory domain. In some aspects, the transmembrane domain comprises a transmembrane domain of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, CD19, or any combination thereof. In some aspects, the transmembrane domain comprises a CD28 transmembrane domain. In some aspects, the intracellular signaling domain comprises an intracellular signaling domain derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcεRI, CD66d, CD32, DAP10, DAP12, or any combination thereof. In some aspects, the intracellular signaling domain comprises a CD3 zeta intracellular signaling domain.

In some aspects, the antigen receptor comprises an engineered TCR. In some aspects, the engineered TCR specifically binds a tumor antigen/MHC complex. In some aspects, the tumor antigen is derived from AFP, CD19, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, neoantigen, or any combinations thereof.

In some aspects, the exogenous polynucleotide comprises a regulatory element, and wherein a vector comprises the exogenous polynucleotide. In some aspects, the vector is a polycistronic expression vector. In some aspects, the regulatory element comprises a promoter. In some aspects, the promoter comprises a dl587rev primer-binding site substituted (MND) promoter, EF1a promoter, ubiquitin promoter, or combinations thereof. In some aspects, the vector comprises a viral vector, a mammalian vector, or a bacterial vector. In some aspects, the vector comprises an adenoviral vector, a lentivirus, a Sendai virus vector, a baculoviral vector, an Epstein Barr viral vector, a papovaviral vector, a vaccinia viral vector, a herpes simplex viral vector, a hybrid vector, or an adeno associated virus (AAV) vector. In some aspects, the vector is a lentivirus.

In some aspects, the concentration of potassium ion is higher than about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, or about 90 mM. In some aspects, the concentration of potassium ion is selected from the group consisting of about 40 mM, about 45 mM, about 50 mM, about 55 mM, about 60 mM, about 65 mM, about 70 mM, about 75 mM, and about 80 mM. In some aspects, the concentration of potassium ion is between about 30 mM and about 80 mM, about 40 mM and about 80 mM, about 50 mM and 80 mM, about 60 mM and about 80 mM, about 70 mM and about 80 mM, about 40 mM and about 70 mM, about 50 mM and about 70 mM, about 60 mM and about 70 mM, about 40 mM and about 60 mM, about 50 mM and about 60 mM, or about 40 mM and about 50 mM. In some aspects, the concentration of potassium ion is about 50 mM, about 60 mM, or about 70 mM.

In some aspects, the medium further comprises sodium ion. In some aspects, the medium further comprises NaCl. In some aspects, the medium comprises less than about 140 mM, about 130 mM, about 120 mM, about 110 mM, about 100 mM, about 90 mM, about 80 mM, about 70 mM, about 60 mM, about 50 mM, or about 40 mM NaCl.

In some aspects, the medium is hypotonic or isotonic. In some aspects, the sum of the potassium ion concentration and the NaCl concentration, multiplied by two is less than 280. In some aspects, the sum of the potassium ion concentration and the NaCl concentration, multiplied by two is more than 240 and less than 280. In some aspects, the sum of the potassium ion concentration and the NaCl concentration, multiplied by two is more than or equal to 280 and less than 300. In some aspects, the concentration of potassium ion is about 60 mM, and the concentration of NaCl is less than 80 mM, less than 75 mM, less than 70 mM, less than 65 mM, or less than 60 mM. In some aspects, the concentration of potassium ion is about 55 mM, and the concentration of NaCl is less than 85 mM, less than 80 mM, less than 75 mM, less than 70 mM, or less than 65 mM. In some aspects, the concentration of potassium ion is about 50 mM, and the concentration of NaCl is less than 90 mM, less than 85 mM, less than 80 mM, less than 75 mM, or less than 70 mM.

In some aspects, the medium further comprises one or more cytokines. In some aspects, the one or more cytokines comprise interleukin-2 (IL-2), interleukin-7 (IL-7), interleukin-21 (IL-21), interleukin-15 (IL-15), or any combination thereof. In some aspects, the one or more cytokines comprise IL-2, IL-7, and IL-15.

In some aspects, the medium further comprises calcium ion, glucose, or both calcium ion and glucose.

In some aspects, the medium further comprises a cell expansion agent. In some aspects, the cell expansion agent comprises a GSK3B inhibitor, an ACLY inhibitor, a PI3K inhibitor, an AKT inhibitor, or any combination thereof. In some aspects, the PI3K inhibitor is selected from hydroxyl citrate, LY294002, pictilisib, CAL101, IC87114, and any combination thereof. In some aspects, the AKT inhibitor is selected from MK2206, A443654, AKTi-VIII, and any combination thereof.

In some aspects, the medium is capable of: (a) increasing the number and/or percentage of less differentiated and/or undifferentiated cells; (b) increasing transduction efficiency; (c) increasing stem-like immune cells; (d) increasing in vivo viability; (e) increasing cell potency; (f) preventing cell exhaustion; (g) increasing the number and/or percentage of effector-like cells; or (h) any combination thereof; in the final cell product as compared to the starting immune cells and/or the immune cells cultured in a medium without the high concentration of potassium ion.

In some aspects, the medium further comprises glucose. In some aspects, the concentration of glucose is more than about 10 mM. In some aspects, the concentration of glucose is from about 10 mM to about 25 mM, about 10 mM to about 20 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of glucose is about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM. In some aspects, the concentration of glucose is about 15.4 mM, about 15.9 mM, about 16.3 mM, about 16.8 mM, about 17.2 mM, or about 17.7 mM.

In some aspects, the medium further comprises calcium ion. In some aspects, the concentration of calcium ion is more than about 0.4 mM. In some aspects, the concentration of calcium ion is from about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM. In some aspects, the concentration of calcium ion is about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

In some aspects, the medium comprises IL-2 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-2 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-2 is about 1.0 ng/mL. In some aspects, the concentration of IL-2 is about 10 ng/mL.

In some aspects, the medium comprises IL-21 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-21 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-21 is about 1.0 ng/mL. In some aspects, the concentration of IL-21 is about 10 ng/mL.

In some aspects, the medium comprises IL-7 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-7 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-7 is about 1.0 ng/mL. In some aspects, the concentration of IL-7 is about 10 ng/mL.

In some aspects, the medium comprises IL-15 at a concentration from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL. In some aspects, the concentration of IL-15 is about 0.1 ng/mL, about 0.5 ng/mL, about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, or about 20 ng/mL. In some aspects, the concentration of IL-15 is about 1.0 ng/mL. In some aspects, the concentration of IL-15 is about 10 ng/mL.

Some aspects of the present disclosure are directed to a population of human immune cells prepared by a method disclosed herein. In some aspects, the human immune cells comprise T cells. In some aspects, the immune cells are CD3⁺, CD45RO⁻, CCR7⁺, CD45RA⁺, CD62L⁺, CD27⁺, CD28⁺, TCF7⁺, or any combination thereof. In some aspects, at least about 10% to at least about 70% of the total number of T cells in the population of human immune cells are stem-like T cells. In some aspects, at least about 10% to at least about 40% of the total number of T cells in the population of human immune cells are CD39⁻/CD69⁻ T cells. In some aspects, at least about 10% to at least about 70% of the total number of T cells in the population of human immune cells are CD39⁻/TCF7+ T cells. In some aspects, the population of human immune cells comprises CD8⁺ T cells.

Some aspects of the present disclosure are directed to a pharmaceutical composition comprising a population of human immune cells disclosed herein, and a pharmaceutically acceptable carrier.

Some aspects of the present disclosure are directed to a method of killing target cells, comprising contacting the target cells with a population of immune cells disclosed herein or a pharmaceutical composition disclosed herein under conditions that allow killing of the target cells by the immune cells.

Some aspects of the present disclosure are directed to a method of treating a patient in need thereof, comprising administering a population of human immune cells disclosed herein or a pharmaceutical composition disclosed herein to the patient.

Some aspects of the present disclosure are directed to use of a population of human immune cells disclosed herein for the manufacture of a medicament for treating a patient in need thereof in a method of disclosed herein.

### BRIEF DESCRIPTION OF THE DRAWINGS/FIGURES

FIG. 1 shows the fold expansion of combined CD4⁺ and CD8⁺ T cells at the end of an 8-day production process. The fold expansion was calculated by dividing the total number of T cells on day 8 by the total number of T cells on day 0. Each dot represents a donor from a study. The data were pooled from three independent studies.

FIG. 2 shows the percentage of CD4⁺ or CD8⁺ T cells within the EGFR⁺ROR1 CAR-T cell population at the end of the 8-day production process. Mean and standard deviation were shown. The data were pooled from three independent studies.

FIGS. 3A-3C show the phenotypic analysis of stem-like CAR-T cells, as defined by CD45RA+ and CCR7+, at the end of the 8-day production process (FIG. 3A). Percentage of stem-like CAR-T cells within all CD3+ EGFR+ CAR-T cells (FIG. 3B). Percentage of stem-like CAR-T cells within CD4+ EGFR+ CAR-T cells (FIG. 3C). Percentage of stem-like CAR-T cells within CD8+ EGFR+ CAR-T cells. Each dot represents a donor from a study. The data were pooled from two independent studies.

FIG. 4 shows the phenotypic analysis of stem-like CAR-T cells, as defined by a more stringent gating strategy (CCR7+ CD45RA+ CD62L+ CD45RO−) across 5 independent donors. Cells were gated on all CD3+ EGFR+ CAR-T cells at the end of the 8-day production process. Each bar indicates the percentage of stem-like T cells within all CAR-T cells.

FIGS. 5A-5E show the intracellular cytokine expression of anti-ROR1 CAR T cells in response to target cell stimulation. FIG. 5A shows a representative flow cytometry plot of intracellular IL-2 and IFN-gamma (IFNg) and gating a strategy for intracellular cytokine analysis, with quadrants B, C, D, and E corresponding to FIGS. 5B, 5C, 5D, and 5E, respectively. T cells were first gated on live EGFR+ CD45+ CD3+ T CAR-T cells, and subsequently gated by IFNg and IL-2 expression. FIG. 5Bs shows the percentage of polyfunctional CAR-T cells, as defined by T cells expressing both IFNg and IL-2, within CD3+ CAR-T cells. FIG. 5C shows the percentage of CAR-T cells that express only IL-2. FIG. 5D shows the percentage of CAR-T cells that express only IFNg. FIG. 5E shows the percentage of “non-functional” CAR-T cells, as defined by those that express neither IL-2 nor IFNg in response to target cell stimulation. Each symbol represents a unique donor.

FIGS. 6A-6F are target cell clearance curves in response to CAR-T cells in an in vitro sequential stimulation assay. Target cells are visualized and quantified using the constitutively expressed fluorescent protein NLR. The amount of viable target cells in the culture is defined as the total NLR intensity and expressed as “total target cell intensity.” A reduction in the total target cell intensity signifies target cell death. FIGS. 6A-6C show the kinetics of target cell death over time during sequential stimulation in response to CAR-T cells produced using the transact process (TA), the PCS 0.3% aCD3/28, or the PCS 0.5% aCD3/28 process, for three different donors, respectively (Donor 6 (FIG. 6A), Donor 7 (FIG. 6B), and Donor 8 (FIG. 6C)). FIGS. 6D-6F show the kinetics of target cell death over time during sequential stimulation in response to CAR-T cells produced using the transact process (TA), the PCS 0.5% aCD3/28, the PCS 0.75% aCD3/28 or the PCS 1% aCD3/28 process, for the three different donors, respectively (Donor 6 (FIG. 6D), Donor 7 (FIG. 6E), and Donor 8 (FIG. 6F)).

FIGS. 7A-7C are target cell clearance curves in response to CAR-T cells using an in vitro potency assay at low effector to target (E:T) ratios (here 1:125, i.e. for every 125 target cells there is 1 CAR-T cell) for three different donors, respectively (Donor 6 (FIG. 7A), Donor 7 (FIG. 7B), and Donor 8 (FIG. 7C)). Target cells were visualized and quantified using the constitutively expressed fluorescent protein NLR. The amount of viable target cells in the culture is defined as the total NLR intensity and expressed as “total target cell intensity.” A reduction in the total target cell intensity signified target cell death. The graphs show kinetics of target cell death over time during sequential stimulation in response to CAR-T cells produced using a mock transact process (TA), the TA process, a mock PCS process, and the PCS 0.5% aCD3/28 process.

FIGS. 8A-8C show the cumulative number of CAR-T cells in response to target cell stimulation in an in vitro sequential stimulation assay for three different donors, respectively (Donor 6 (FIG. 8A), Donor 7 (FIG. 8B), and Donor 8 (FIG. 8C)). The number of CAR-T cells was obtained by multiplying the percentage of live EGFR+ CD45+ NLR− cells by the total number of cells in the well.

FIG. 9 is a graphical representation of the fold expansion of combined CD4+ and CD8+ T cells at the end of an 8-day production process. The fold expansion was calculated by dividing the total number of T cells on day 8 by the total number of T cells on day 0. Each dot represents a unique donor.

FIG. 10 is a bar graph showing the phenotypic analysis of stem-like CAR-T cells, as defined by CCR7+ CD45RA+ CD62L+ CD45RO− expression, across 3 independent donors. Cells were gated on all CD3+ EGFR+ CAR-T cells at the end of the 8-day production process. Each bar indicates the percentage of stem-like T cells within all CAR-T cells.

FIGS. 11A-11B show the intracellular cytokine expression of anti-ROR1 CAR T cells in response to target cell stimulation. FIG. 11A shows the percentage of polyfunctional CAR-T cells, as defined by T cells expressing both IFNg and IL-2 following target cell stimulation, within CD3+ CAR-T cells. FIG. 11B shows the percentage of “non-functional” CAR-T cells, as defined by those that express neither IL-2 nor IFNg following target cell stimulation. Each symbol represents a unique donor.

FIGS. 12A-12C are target cell clearance curves in response to CAR-T cells in an in vitro sequential stimulation assay in three independent donors, respectively (Donor 7 (FIG. 12A), Donor 8 (FIG. 12B), and Donor 9 (FIG. 12C)). Target cells are visualized and quantified using the constitutively expressed fluorescent protein NLR. The amount of viable target cells in the culture is defined as the total NLR intensity and expressed as “total target cell intensity.” A reduction in the total target cell intensity signifies target cell death.

FIGS. 13A-13C are target cell clearance curves in response to CAR-T cells in an in vitro serial stimulation assay in three independent donors, respectively (Donor 7 (FIG. 13A), Donor 8 (FIG. 13B), and Donor 9 (FIG. 13C)). Target cells are visualized and quantified using its constitutively expressed fluorescent protein NLR. The amount of viable target cells in the culture is defined as the total NLR intensity and expressed as “total target cell intensity”. A reduction in the total target cell intensity signifies target cell death.

FIGS. 14A-14F are graphical representations of cell activation (FIGS. 14A-14B), expansion (FIGS. 14C-14D), and transduction (FIGS. 14A-14F) for cells cultured in control (TCM) and various MRM (MRM-1, MRM-2, MRM-3, MRM-4, and standard MRM) cultures and activated with a first PCS (0.5% density, 1:1; FIGS. 14A, 14C, and 14E) or a second PCS (0.1% density, 1:1; FIGS. 14B, 14D, and 14F) composition.

### DETAILED DESCRIPTION

The efficacy of cellular immunotherapy is dependent on a number of factor including the persistence, multipotency, and asymmetric cell division of the cell product that is infused in to the patient. The media and methods used in culturing and/or engineering of the cells used for cell therapy can profoundly affect the metabolic, epigenetic, and phenotypic attributes of these cells thereby affecting their therapeutic potential.

The present disclosure is directed to methods of culturing cells, cells prepared by the methods, and/or compositions or kits for the cell culturing methods. Some aspects of the present disclosure are directed to methods of preparing a population of human immune for immunotherapy, methods of activating a population of human immune cells for immunotherapy, methods of increasing the yield of activated human immune cells during ex vivo or in vitro culture, methods of increasing stemness of activated human immune cells while increasing the yield of activated human immune cells during ex vivo or in vitro culture for an immunotherapy, and methods of expanding a population of activated stem-like immune cells ex vivo or in vitro; comprising contacting immune cells with programmable cell-signaling scaffolds (PCS) in a medium comprising potassium ion at a concentration higher than 5 mM.

In some aspects, the disclosure provides methods of generating a population of immune cells, e.g., T cells or NK cells, for adoptive cell therapy (ACT), wherein the immune cells, e.g., T cells or NK cells, have a less differentiated state and retain the ability to proliferate. In some aspects, the immune cells, e.g., T cells or NK cell, have a less differentiated state and maintain the ability to target and kill tumor cells. In some aspects, the immune cells, e.g., T cells or NK cell, have a less differentiated state, retain the ability to proliferate, and maintain the ability to target and kill tumor cells. In some aspects, immune cells, e.g., T cells or NK cell, cultured according to the methods disclosed herein, have increased efficacy in ACT, as compared to cells cultured according to conventional methods, e.g., in a medium having less than 5 mM potassium ion. In some aspects, immune cells, e.g., T cells or NK cell, cultured according to the methods disclosed herein, have increased persistence upon administration to a subject in ACT, as compared to immune cells cultured according to conventional methods, e.g., in a medium having less than 5 mM potassium ion. Such increased persistence refers to the ability of the immune cell, e.g., T cells or NK cell, to infiltrate and function in the tumor microenvironment, ability to resist exhaustion, and the persistence of stemness to ensure continued expansion and durability of response. In some aspects, immune cells, e.g., T cells or NK cell, cultured according to the methods disclosed herein, are stem-like cells. Such cells are capable of self-renewal, proliferation and differentiation. In some aspects, immune cells, e.g., T cells or NK cell, cultured according to the methods disclosed herein, are stem-like cells which also express effector-like markers. In some aspects, immune cells, e.g., T cells or NK cell, cultured according to the methods disclosed herein, are stem-like cells which also maintain the ability to target and kill tumor cells.

The cell culturing methods of the present disclosure are capable of increasing multipotency and/or pluripotency of the cultured cells or increasing transduction efficiency when the cells are being transduced with a vector. In some aspects, the culturing methods are capable of reducing and/or preventing cell exhaustion when the cells are cultured and/or the cells are used in therapy in vivo. In some aspects, the culturing methods are also capable of increasing in vivo viability, in vivo persistence, in vivo effector function, or any combination thereof. In some aspects, the culturing methods disclosed herein are capable of enriching oligoclonal or polyclonal tumor reactive stem-like T-cells and/or CD8⁺ TILs. In some aspects, the culturing methods disclosed herein are capable of preserving clonal diversity of the TILs derived from cancer patients.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order that is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

Throughout the disclosure, the term “a” or “an” entity refers to one or more of that entity; for example, “a chimeric polypeptide,” is understood to represent one or more chimeric polypeptides. As such, the terms “a” (or “an”), “one or more,” and “at least one” can be used interchangeably herein. In addition, “or” is used to mean an open list of the components in the list. For example, “wherein X comprises A or B” means X comprises A, X comprises B, X comprises A and B, or X comprises A or B and any other components.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range, unless otherwise explicitly stated.

Abbreviations used herein are defined throughout the present disclosure. Various aspects of the disclosure are described in further detail in the following subsections.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% (e.g., a range of values that fall within 10%, 9%, 8%, 7%, 6%, 5%, 4%, 3%, 2%, 3% 2%, 1%, or less in either direction (greater than or less than) of the stated reference value unless otherwise stated or otherwise evident from the context (except where such number would exceed 100% of a possible value)). For example, “about 55 mM,” as used herein, includes 49.5 mM to 60.5 mM. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As used herein, the term “approximately,” as applied to one or more values of interest, refers to a value that is similar to a stated reference value. In some aspects, the term “approximately,” like the term, “about,” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As described herein, any concentration range, percentage range, ratio range, or integer range is to be understood to include the value of any integer within the recited range and, when appropriate, fractions thereof (such as one tenth and one hundredth of an integer), unless otherwise indicated.

The term “control media,” “conventional culture media,” or “reference culture media” as used herein refers to any media in comparison to a metabolic reprogramming media (MRM) disclosed herein. Control media can comprise the same components as the metabolic reprogramming media except certain ion concentrations, e.g., potassium ion. In some aspects, metabolic reprogramming media described herein are prepared from control media by adjusting one or more ion concentrations, e.g., potassium ion concentration, as described herein. In some aspects, control media comprise basal media, e.g., CTS™ OPTMIZER™. In some aspects, control media thus comprises one or more additional components, including, but not limited to, amino acids, glucose, glutamine, T cell stimulators, antibodies, substituents, etc. that are also added to the metabolic reprogramming media, but control media have certain ion concentrations different from the metabolic reprogramming media. In some aspects, the control media does not comprise programmable cell-signaling scaffolds (PCS), as disclosed herein. Unless indicated otherwise, the terms “media” and “medium” can be used interchangeably.

The term “culturing” as used herein refers to the controlled growth of cells ex vivo and/or in vitro. As used herein, “culturing” includes the growth of cells, e.g., immune cells, e.g., one or more engineered immune cell disclosed herein, during cell expansion, or cell engineering (e.g., transduction with a construct for expressing a CAR, a TCR, or a TCRm). In some aspects, the cultured cells are obtained from a subject, e.g., a human subject/patient. In some aspects, the cultured cells comprise immune cells obtained from a human subject. In some aspects, the cultured cells comprise one or more engineered immune cell disclosed herein. In some aspects, the cultured cells comprise T cells or NK cells obtained from a human subject/patient. In some aspects, the T cells and/or NK cells are purified prior to the culture. In some aspects, the T cells and/or NK cells are tumor-infiltrating T cells and/or NK cells. In some aspects, the cultured cells comprise one or more engineered immune cell disclosed herein.

The term “expand” or “expansion,” as used herein in reference to immune cell culture refers to the process of stimulating or activating the cells and culturing the cells. The expansion process can lead to an increase in the proportion or the total number of desired cells, e.g., an increase in the proportion or total number of less differentiated immune cells, in a population of cultured cells, after the cells are stimulated or activated and cultured. Expansion does not require that all cell types in a population of cultured cells are increased in number. Rather, in some aspects, only a subset of cells in a population of cultured cells are increased in number during expansion, while the number of other cell types may not change or may decrease.

As used herein, the term “yield” refers to the total number of cells following a culture method or a portion thereof. In some aspects, the term “yield” refers to a particular population of cells, e.g., stem-like T cells in a population of T cells. The yield can be determined using any methods, including, but not limited to, estimating the yield based on a representative sample.

As used herein, the term “metabolic reprogramming media,” “metabolic reprogramming medium,” or “MRM,” refers to a medium of the present disclosure, wherein the medium has an increased potassium concentration. In some aspects, the metabolic reprogramming media comprises potassium ion at a concentration higher than 5 mM. In some aspects, the metabolic reprogramming media comprises potassium ion at a concentration higher than 40 mM. In some aspects, the metabolic reprogramming media comprises a concentration of potassium ion of at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM. In some aspects, the metabolic reprogramming media comprises about 40 mM to about 80 mM NaCl, about 40 mM to about 90 mM KCl, about 0.5 mM to about 2.8 mM calcium, and about 10 mM to about 24 mM glucose. In some aspects, the metabolic reprograming media further comprises an osmolality of about 250 to about 300 mOsmol. In some aspects, the metabolic reprogramming medium further comprises programmable sell-signaling scaffolds (PCS), as disclosed herein.

As used herein, the term “higher than” means greater than but not equal to. For example, “higher than 4 mM” means any amount that is more than 4 mM, but which does not include 4 mM.

As used herein, the term “tonicity” refers to the calculated effective osmotic pressure gradient across a cell membrane, represented by the sum of the concentration of potassium ion and the concentration of sodium chloride (NaCl), multiplied by two. Tonicity can be expressed in terms of the osmolality (mOsm/kg) or osmolarity (mOsm/L) of the solution, e.g., the media. Osmolality and osmolarity are measurements of the solute osmotic concentration of a solvent per mass (osmolality) and per volume (osmolarity). As used herein, an isotonic medium has a tonicity of about 280 mOsm/L (e.g., ([K+]+[NaCl])×2=280).

As used herein, a hypotonic solution has a tonicity of less than 280 mOsm/L (e.g., ([K+]+[NaCl])×2<280). In some aspects, a hypotonic medium has a tonicity from at least about 210 mOsm/L to less than about 280 mOsm/L. In some aspects, a hypotonic medium has a tonicity from at least about 220 mOsm/L to less than about 280 mOsm/L. In some aspects, a hypotonic medium has a tonicity from at least about 230 mOsm/L to less than about 280 mOsm/L. In some aspects, a hypotonic medium has a tonicity from at least about 240 mOsm/L to less than about 280 mOsm/L. In some aspects, a hypotonic medium described herein has a tonicity of about 250 mOsm/L.

As used herein, a hypertonic solution has a tonicity of greater than 300 mOsm/L (e.g., ([K+]+[NaCl])×2>300). In some aspects, a hypertonic medium described herein has a tonicity of about 320 mOsm/L. In some aspects, the tonicity of the solution, e.g., medium is adjusted by increasing or decreasing the concentration of potassium ions and NaCl. In some aspects, the tonicity of a medium can be maintained by offsetting the increase of one solute with a decrease in a second solute. For example, increasing the concentration of potassium ion in a medium without changing the concentration of sodium ions can increase the tonicity of the medium. However, if the concentration of potassium ions is increased and the concentration of sodium ions is decreased, the tonicity of the original medium can be maintained.

As used herein, the terms “potassium,” “potassium ion,” “potassium cation,” and “K+” are used interchangeably to refer to elemental potassium. Elemental potassium exists in solution as a positive ion. However, it would be readily apparent to a person of ordinary skill in the art that standard means of preparing a solution comprising potassium ion include diluting a potassium containing salt (e.g., KCl) into a solution. As such, a solution, e.g., a medium, comprising a molar (M) concentration of potassium ion, can be described as comprising an equal molar (M) concentration of a salt comprising potassium.

As used herein, the terms “sodium ion” and “sodium cation” are used interchangeably to refer to elemental sodium. Elemental sodium exists in solution as a monovalent cation. However, it would be readily apparent to a person of ordinary skill in the art that standard means of preparing a solution comprising sodium ion include diluting a sodium-containing salt (e.g., NaCl) into a solution. As such, a solution, e.g., a medium, comprising a molar (M) concentration of sodium ion, can be described as comprising an equal molar (M) concentration of a salt comprising sodium.

As used herein, the terms “calcium ion” and “calcium cation” are used interchangeably to refer to elemental calcium. Elemental calcium exists in solution as a divalent cation. However, it would be readily apparent to a person of ordinary skill in the art that standard means of preparing a solution comprising calcium ion include diluting a calcium-containing salt (e.g., CaCl₂)) into a solution. As such, a solution, e.g., a medium, comprising a molar (M) concentration of calcium ion, can be described as comprising an equal molar (M) concentration of a salt comprising calcium.

As used herein, the term “immune cell” refers to a cell of the immune system. In some aspects, the immune cell is selected from a T lymphocyte (“T cell”), B lymphocyte (“B cell”), natural killer (NK) cell, natural killer T lymphocytes (NKT cells), macrophage, eosinophil, mast cell, dendritic cell or neutrophil. As used herein, a “population” of cells refers to a collection of more than one cell, e.g., a plurality of cells. In some aspects, the population of cells comprises more than one immune cell, e.g., a plurality of immune cells. In some aspects, the population of cells is comprises a heterogeneous mixture of cells, comprising multiple types of cells, e.g., a heterogeneous mixture of immune cells and non-immune cells. In some aspects, the population of cells comprises a plurality of T cells.

As used herein, the terms “T cell” and “T lymphocyte” are interchangeable and refer to any lymphocytes produced or processed by the thymus gland. Non-limiting classes of T cells include effector T cells and T helper (Th) cells (such as CD4⁺ or CD8⁺ T cells). In some aspects, the T cell is a Th1 cell. In some aspects, the T cell is a Th2 cell. In some aspects, the T cell is a Tc17 cell. In some aspects, the T cell is a Th17 cell. In some aspects, the T cell is a T_{reg} cell. In some aspects, the T cell is a tumor-infiltrating cell (TIL).

As used herein, the term “memory” T cells refers to T cells that have previously encountered and responded to their cognate antigen (e.g., in vivo, in vitro, or ex vivo) or which have been stimulated, e.g., with an anti-CD3 antibody (e.g., in vitro or ex vivo). Immune cells having a “memory-like” phenotype upon secondary exposure, such memory T cells can reproduce to mount a faster and strong immune response than during the primary exposure. In some aspects, memory T cells comprise central memory T cells (T_{CM} cells), effector memory T cells (T_{EM} cells), tissue resident memory T cells (T_{RM} cells), stem cell-like memory T cells (T_{SCM} cells), or any combination thereof.

As used herein, the term “stem cell-like memory T cells,” “T memory stem cells,” or “T_{SCM} cells” refers to memory T cells that express CD95, CD45RA, CCR7, and CD62L and are endowed with the stem cell-like ability to self-renew and the multipotent capacity to reconstitute the entire spectrum of memory and effector T cell subsets.

As used herein, the term “central memory T cells” or “T_{CM} cells” refers to memory T cells that express CD45RO, CCR7, and CD62L. Central memory T cells are generally found within the lymph nodes and in peripheral circulation.

As used herein, the term “effector memory T cells” or “T_{EM} cells” refers to memory T cells that express CD45RO but lack expression of CCR7 and CD62L. Because effector memory T cells lack lymph node-homing receptors (e.g., CCR7 and CD62L), these cells are typically found in peripheral circulation and in non-lymphoid tissues.

As used herein, the term “tissue resident memory T cells” or “T_{RM} cells” refers to memory T cells that do not circulate and remain resident in peripheral tissues, such as skin, lung, and gastrointestinal tract. In some aspects, tissue resident memory T cells are also effector memory T cells.

As used herein, the term “naïve T cells” or “T_{N} cells” refers to T cells that express CD45RA, CCR7, and CD62L, but which do not express CD95. T_{N} cells represent the most undifferentiated cell in the T cell lineage. The interaction between a T_{N} cell and an antigen presenting cell (APC) induces differentiation of the T_{N} cell towards an activated TEFF cell and an immune response.

As used herein, the term “stemness,” “stem cell-like,” “stem-like,” or “less-differentiated” refers to an immune cell (e.g., a T cell or an NK cell), that expresses markers consistent with a more naïve phenotype. For example, a less differentiated T cell can express one or more marker characteristic of a T_{N} or a T_{SCM} cell. In some aspects, a “less-differentiated” or “stem-like” T cell expresses CD45RA, CCR7, and CD62L. In some aspects, a “less-differentiated” or “stem-like” T cell expresses CD45RA, CCR7, CD62L, and TCF7. In some aspects, a “less-differentiated” or “stem-like” T cell does not express CD45RO or is CD45RO^{low}. In some aspects, the methods disclosed herein promote immune cells (e.g., T cells and/or NK cells) having a less-differentiated phenotype. Without being bound by any particular mechanism, in some aspects, the methods disclosed herein block, inhibit, or limit differentiation of less-differentiated immune cells (e.g., T cells and/or NK cells), resulting in an increased number of stem-like cells in culture. For example, it is generally thought that to effectively control tumors, adoptive transfer of less-differentiated immune cells, e.g., T cells and/or NK cells, with a stem cell-like memory or central memory phenotype are preferred. See Gattinoni, L., et al., J. Clin. Invest. 115:1616-1626 (2005), Gattinoni, L., et al. Nat Med 15(7):808-814 (2009), Lynn, R. C., et al., Nature 576(7786): 293-300 (2019); Gattinoni, L., et al. Nat Rev 12:671-684 (2012), Klebanoff, C., et al., J. Immunother 35(9):651-670 (2012) and Gattinoni, L., et al., Nat Med 17(10): 1290-1297 (2011).

Stemness is characterized by the capacity to self-renew, the multipotency, and the persistence of proliferative potential. In some aspects, stemness is characterized by a particular gene signature, e.g., a combined pattern of expression across a multitude of genes. In some aspects, the stem-like cells can be identified by a transcriptome analysis, e.g., using stemness gene signatures disclosed herein. In some aspects, the gene signature comprises one or more genes selected from ACTN1, DSC1, TSHZ2, MYB, LEF1, TIMD4, MAL, KRT73, SESN3, CDCA7L, LOC283174, TCF7, SLC16A10, LASS6, UBE2E2, IL7R, GCNT4, TAF4B, SULT1B1, SELP, KRT72, STXBP1, TCEA3, FCGBP, CXCR5, GPA33, NELL2, APBA2, SELL, VIPR1, FAM153B, PPFIBP2, FCER1G, GJB6, OCM2, GCET2, LRRN1, IL6ST, LRRC16A, IGSF9B, EFHA2, LOC129293, APP, PKIA, ZC3H12D, CHMP7, KIAA0748, SLC22A17, FLJ13197, NRCAM, C5orf13, GIPC3, WNT7A, FAM117B, BEND5, LGMN, FAM63A, FAM153B, ARHGEF11, RBM11, RIC3, LDLRAP1, PELI1, PTK2, KCTD12, LMO7, CEP68, SDK2, MCOLN3, ZNF238, EDAR, FAM153C, FAAH2, BCL9, C17orf48, MAP1D, ZSWIM1, SORBS3, IL4R, SERPINFI, C16orf45, SPTBN1, KCNQ1, LDHB, BZW2, NBEA, GAL3ST4, CRTC3, MAP3K1, HLA-DOA, RAB43, SGTB, CNN3, CWH43, KLHL3, PIM2, RGMB, C16orf74, AEBP1, SNORD115-11, SNORD115-11, GRAP, and any combination thereof (see, e.g., Gattinoni et al., Nature Medicine 17(10):1290-97 (2011)). In some aspects, the gene signature comprises one or more gene selected from NOG, TIMD4, MYB, UBE2E2, FCER1G, HAVCR1, FCGBP, PPFIBP2, TPST1, ACTN1, IGF1R, KRT72, SLC16A10, GJB6, LRRN1, PRAGMIN, GIPC3, FLNB, ARRB1, SLC7A8, NUCB2, LRRC7, MYO15B, MAL, AEBP1, SDK2, BZW2, GAL3ST4, PITPNM2, ZNF496, FAM117B, C16orf74, TDRD6, TSPAN32, C18orf22, C3orf44, LOC129293, ZC3H12D, MLXIP, C7orf10, STXBP1, KCNQ1, FLJ13197, LDLRAP1, RAB43, RIN3, SLC22A17, AGBL3, TCEA3, NCRNA00185, FAM153B, FAM153C, VIPR1, MMP19, HBS1L, EEF2K, SNORA5C, UBASH3A, FLJ43390, RP6-213H19.1, INPP5A, PIM2, TNFRSF10D, SNRK, LOC100128288, PIGV, LOC100129858, SPTBN1, PROS1, MMP28, HES1, CACHD1, NSUN5C, LEF1, TTTY14, SNORA54, HSF2, C16orf67, NSUN5B, KIAA1257, NRG2, CAD, TARBP1, STRADB, MT1F, TMEM41B, PDHX, KDM6B, LOC100288322, UXS1, LGMN, NANOS2, PYGB, RASGRP2, C14orf80, XPO6, SLC24A6, FAM113A, MRM1, FBXW8, NDUFS2, KCTD12, and any combination thereof (see, e.g., Gattinoni, L., et al., Nat Med 17(10): 1290-1297 (2011)). In some aspects, the gene signature comprises one or more gene selected from SELL, CCR7, S1PR1, KLF3, TCF7, GPR183, SC5D, FAAH2, LTB, SESN3, MAL, TSHZ2, LEF1, AP3M2, SLC2A3, ICAM2, PLAC8, SCML1, IL7R, ABLIM1, RASGRP2, TRABD2A, SATB1, ALG13, ARID5A, BACH2, PABPC1, GPCPD1, NELL2, TAF4B, FCMR, ARRDC2, C1orf162, FAM177A1, ANKRD12, TXK, SORL1, AQP3, ADTRP, FXYD7, CD28, P2RY8, CRYBG1, TNFSF8, BEX2, PGAP1, PTGER4, MAML2, BEX3, PCSK1N, INPP4B, AC119396.1, CXCR5, LINC00402, CCR4, IL6R, ZBTB10, ITGA6, ARMH1, RILPL2, FOXP1, TESPA1, YPEL5, LPAR6, CMSS1, RIPOR2, ZNF331, EMP3, GIMAP7, WDR74, RIC3, CYSLTR1, ITGB1, CD5, SAMHD1, SERINC5, and any combination thereof (see e.g., Caushi et al., Nature 596: 126-132 (2021)).

As used herein, the term “effector-like” or “effector cell-like” refers to tumor cell killing capacity and cytokine polyfunctionality, e.g., ability of a cell to produce inflammatory cytokines and/or cytotoxic molecules. In some aspects, an effector-like cell is characterized by specific markers expressed by the cell. In some aspects, those effector-like markers comprise one or more of pSTAT5+, STAT5+, pSTAT3+, and STAT3+. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL11RA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof. In some aspects, the effector-like cells are characterized by a transcriptome analysis. In some aspects, the effector-like marker comprises a marker disclosed in Kaech et al., Cell 111:837-51 (2002); Tripathi et al., J. Immunology 185:2116-24 (2010); and/or Johnnidis et al., Science Immunology 6: eabe3702 (Jan. 15, 2021), each of which is incorporated by reference herein in its entirety.

In some aspects, the effector-like cells are characterized using an effector-associated gene set described in Gattinoni, L., et al., Nat Med 17(10):1290-97 (2011). In some aspects, the gene signature for effector-like cells comprises one or more genes selected from MTCH2, RAB6C, KIAA0195, SETD2, C2orf24, NRD1, GNA13, COPA, SELT, TNIP1, CBFA2T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CUP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMI, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf21, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, KLRB1, and any combination thereof (see, e.g., Gattinoni, L., et al., Nat Med 17(10):1290-97 (2011).

In some aspects, the characteristics of a cell (e.g., T cells and/or NK cells) can be assessed using transcriptome analysis by comparing the upregulation and/or downregulation of different set of genes associated with T cell activation (also referred to herein as “TACT genes”), T cell progenitor exhaustion (also referred to herein as “TPE genes”), T cell terminal exhaustion (also referred to herein as “TTE genes”).

In some aspects, the terminally exhausted T cells are characterized using a TTE-associated gene set described in Oliveira et al., Nature 596: 119-125 (2021). In some aspects, the gene signature for TTE cells comprises one or more or all of the genes selected from: KRT86, RDH10, ACP5, CXCR6, HMOX1, LAYN, CLIC3, HAVCR2, AC243829.4, PRF1, SLC2A8, CHST12, GALNT2, ENTPD1, LAG3, GZMB, PDCD1, CARD16, CTLA4, SLA2, CD27, RALA, VCAM1, SYNGR2, NKG7, LSP1, CCL5, RARRES3, CD7, CTSW, MTSS1, PTMS, BATF, KIR2DL4, AKAP5, CD38, RAB27A, GZMH, IGFLR1, ATP8B4, CD63, HOPX, TNFRSF18, ADGRG1, PLPP1, CSF1, TNFSF10, SNAP47, LINC01871, MYO1E, ZBED2, AHI1, ABI3, FASLG, TYMP, ZBTB38, CTSB, PLSCR1, AFAP1L2, ITGAE, TNS3, DUSP16, CASP1, CARS, DUSP5, IFIT1, SLC1A4, GOLIM4, RSAD2, DNPH1, NBL1, ACOT9, ABHD6, OAS1, SLC27A2, ZBP1, CD200R1, OAS3, CMPK2, TNFSF4, POLR1E, CADM1, HELZ2, SYTL2, AGPAT2, UBE2F, GIMAP6, ZBTB32, RIN3, PLEKHF1, CHPF, PACSIN2, ABCB1, SPATS2L, USP18, TMEM9, KLRC1, MPST. In some aspects, progenitor exhausted T cells (TPE) are characterized using a TPE-associated gene set described in Oliveira et al., Nature 596: 119-125 (2021). In some aspects, the gene signature for TPE cells comprises one or more or all of the genes selected from: FXYD6, CAV1, GNG4, XCL1, CRTAM, CXCL13, GEM, XCL2, FXYD2, HLA-DRA, LANCL2, RASSF4, BAG3, HSPA1B, HLA-DQA1, HSPB1, FABP5, MS4A6A, SERPINH1, HLA-DPA1, HLA-DRB1, HSPA1A, RGS2, DRAIC, CD74, HSPD1, HSPA6, HSPE1, CD82, TOX, CD200, HLA-DPB1, NR4A2, VCAM1, BEX3, AIF1, DNAJA1, HSPH1, DNAJB1, HIPK2, LHFPL6, HLA-DMA, GK, TSHZ2, LPL, C16orf45, ZFAND2A, CD80, ETV1, NMB, DEDD2, CMC1, PON2, SEMA4A, ENC1, GRAMD1A, MYL6B, BCAT1, ARMH1, TIAM1, PIKFYVE, MRPL18, INPP5F, LMCD1, SESN3, CCDC6, KIAA1324, CHN1, ANKRD10, CD70, PRRG4, TNFSF4, CORO1B, DNAJB4, MAGEH1, ICAM1, GGT1, NINJ2, BLVRA, FAAH2, TOX2, SLK, CCDC141, ATF3, INPP1, FAM3C, GADD45G, APP, MAL, SIT1, DRAM1, CLECL1, MDFIC, PMCH, HLA-DMB, PHF6, AFAP1L2, BTN2A2, CCL4L2. In some aspects, activated T cells (TACT) are characterized using a TACT-associated gene set described in Oliveira et al., Nature 596: 119-125 (2021). In some aspects, the gene signature for activated T cells comprises one or more or all of the genes selected from: EGR1, HSPA6, FOS, HSPA1B, GADD45B, NR4A1, FOSB, ATF3, DNAJB1, DUSP1, JUNB, CD69, NR4A2, NFKBIA, PPP1R15A, KLF6, DNAJA1, JUN, SRSF7, SLC2A3, ZFP36L1, IER2, HSPA1A, EIF4A2, ID1, IFRD1, CCNL1, RSRP1, SERTAD1, DEDD2, KLF10, AL118516.1, KLF2, ZFAND2A, CLK1, RSRC2, IER3, BTG2, MYLIP, MAFF, CSRNP1, ID2, ZC3H12A, BAG3, SNHG12, TNF, DDIT4, SGK1, SNHG15, DNAJB4, NR4A3, NFKBID, SCML1, RASD1, ATF4, AREG, RASGEF1B, AC020916.1, DDIT3, SNHG8, CITED2, TXNIP, TOB1, PIM2, SOCS3, GADD45G, RGS16, TIPARP, NFKBIZ, CCL4, CD83, PPP1R10, CCL4L2, SESN2, CHMPIB, LEF1, CSKMT, HEXIM1, HSPA2, MRPL18, RBKS, CD55, ARRDC2, SC5D, FAM53C, ATP2B1-AS1, IFNG, MYC, TSC22D2, SERPINH1, LRIF1, ARRDC3, ILF3-DT, INTS6, ZNF10, PRMT9, ATM, SELL, AC243960.1.

In the presence of prolonged antigen exposure, such as in many cancers, more differentiated immune cells, e.g., effector and effector memory T cells, often become exhausted and lose their anti-tumor function. Biomarkers, e.g., T cell markers, can be measured using any methods. In some aspects, T cells are identified using antibody-staining following by gated flow cytometry.

As used herein, the term “basal” media refers to any starting media that is supplemented with one or more of the additional elements disclosed herein, e.g., potassium, sodium, calcium, glucose, IL-2, IL-7, IL-15, IL-21, programmable cell-signaling scaffolds (PCS), or any combination thereof. The basal media can be any media for culturing immune cells, e.g., T cells and/or NK cells. In some aspects, the basal media comprises a balanced salt solution (e.g., PBS, DPBS, HBSS, EBSS), Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ Pro, OPTMIZER™ CTS™ T-Cell Expansion Basal Medium (ThermoFisher), OPTMIZER™, OPTMIZER™ Complete, IMMUNOCULT™ XF (STEMCELL™ Technologies), AIM V™, TEXMACS™ medium, PRIME-XV® T cell CDM, X-VIVO™ 15 (Lonza), TRANSACT™ TIL expansion medium, or any combination thereof. In some aspects, the basal medium is serum free. In some aspects, the basal media comprises PRIME-XV® T cell CDM. In some aspects, the basal media comprises OPTMIZER™. In some aspects, the basal media comprises OPTMIZER™ Pro. In some aspects, the basal medium further comprises immune cell serum replacement (ICSR). For example, in some aspects, the basal medium comprises OPTMIZER™ Complete supplemented with ICSR, AIM V™ supplemented with ICSR, IMMUNOCULT™ XF supplemented with ICSR, RPMI supplemented with ICSR, TEXMACS™ supplemented with ICSR, or any combination thereof. In some aspects, suitable basal media include Click's medium, OPTMIZER™ (CTS™) medium, STEMLINE® T cell expansion medium (Sigma-Aldrich), AIM V™ medium (CTS™), TEXMACS™ medium (Miltenyi Biotech), IMMUNOCULT™ medium (Stem Cell Technologies), PRIME-XV® T-Cell Expansion XSFM (Irvine Scientific), Iscoves medium, and/or RPMI-1640 medium. In some aspects, the basal media comprises NaCl free CTS™ OPTMIZER™ In some aspects, the basal media comprises one or more sodium salt in addition to the NaCl.

As used herein, the term “cytokine” refers to small, secreted proteins released by cells that have a specific effect on the interactions and communications between cells. Non-limiting examples of cytokines include interleukins (e.g., interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21 and IL-23), interferons (IFN; e.g., IFN-α, IFN-β, and IFN-γ), tumor necrosis factor (TNF) family members, and transforming growth factor (TGF) family members. Some aspects of the present disclosure are directed to methods of culturing and/or expanding immune cells, e.g., T cells and/or NK cells or one or more engineered immune cell disclosed herein, in a medium comprising a cytokine. In some aspects, the cytokine is an interleukin. In some aspects, the cytokine comprises IL-2, IL-7, IL-15, IL-21 or any combination thereof. IL-2 (UniProtKB—P60568) is produced by T cells in response to antigenic or mitogenic stimulation. IL-2 is known to stimulate T cell proliferation and other activities crucial to regulation of the immune response. IL-7 (UniProtKB—P13232) is a hematopoietic growth factor capable of stimulating the proliferation of lymphoid progenitors. IL-7 is believed to play a role in proliferation during certain stages of B-cell maturation. IL-15 (UniProtKB—P40933), like IL-2, is a cytokine that stimulates the proliferation of T-lymphocytes. IL-21 (UniProtKB—Q9HBE4) is a cytokine with immunoregulatory activity. IL-21 is thought to promote the transition between innate and adaptive immunity and to induce the production of IgG1 and IgG3 in B-cells. IL-21 may also play a role in proliferation and maturation of natural killer (NK) cells in synergy with IL-15, and IL-21 may regulate proliferation of mature B- and T-cells in response to activating stimuli. In synergy with IL-15 and IL-18, IL-15 also stimulates interferon gamma production in T-cells and NK cells, and IL-21 may also inhibit dendritic cell activation and maturation during a T-cell-mediated immune response

As used herein, the term “transduction efficiency” refers to: (i) the amount of material (e.g., exogenous polynucleotide) that can be physically introduced into a cell within a defined period of time; (ii) the amount of time it takes to physically introduce a given amount of material into a cell; (iii) the level to which a target material, e.g., an exogenous polynucleotide, i.e., a transgene, is taken up by a population of cells (e.g., the percentage of cells that express the transgene); or (iv) any combination of (i)-(iii). In some aspects, by increasing transduction efficiency, the culturing methods provided herein can allow for a greater amount of an exogenous nucleotide sequence to be introduced into a cell and/or decrease the amount of time required to introduce a given amount of an exogenous nucleotide sequence. Not to be bound by any one theory, in some aspects, such an effect can increase the expression of the encoded protein (e.g., c-Jun polypeptide) in the modified immune cell.

As used herein, “administering” refers to the physical introduction of a therapeutic agent or a composition comprising a therapeutic agent to a subject, using any of the various methods and delivery systems. The different routes of administration for a therapeutic agent described herein (e.g., an immune cell modified to express a chimeric binding protein and a c-Jun polypeptide, and cultured as described herein) include intravenous, intraperitoneal, intramuscular, subcutaneous, spinal or other parenteral routes of administration, for example by injection or infusion.

The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricular, intravitreal, epidural, and intrasternal injection and infusion, as well as in vivo electroporation.

Alternatively, a therapeutic agent described herein (e.g., an immune cell modified to express a chimeric binding protein and/or a TCR that binds a tumo antigen, and cultured as described herein) can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, “cell engineering” or “cell modification” (including derivatives thereof) refers to the targeted modification of a cell, e.g., an immune cell disclosed herein. In some aspects, the cell engineering comprises viral genetic engineering, non-viral genetic engineering, introduction of receptors to allow for tumor specific targeting (e.g., a chimeric binding protein and/or a TCR) introduction of one or more endogenous genes that improve T cell function, introduction of one or more synthetic genes that improve immune cell, e.g., T cell, function (e.g., a polynucleotide encoding a c-Jun polypeptide, such that the immune cell exhibits increased c-Jun expression), or any combination thereof.

As used herein, the term “antigen” refers to any natural or synthetic immunogenic substance, such as a protein, peptide, or hapten. As used herein, the term “cognate antigen” refers to an antigen which an immune cell (e.g., T cell) recognizes and thereby, induces the activation of the immune cell (e.g., triggering intracellular signals that induce effector functions, such as cytokine production, and/or for proliferation of the cell). In some aspects, the antigen comprises a tumor antigen. In some aspects, the antigen comprises a neoantigen.

A “cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein comprises primary, metastatic and recurrent cancers. Unless indicated otherwise, the terms “cancer” and “tumor” can be used interchangeably.

The term “hematological malignancy” or “hematological cancer” refers to mammalian cancers and tumors of the hematopoietic and lymphoid tissues. Non-limiting examples of hematological malignancies include those affecting tissues of the blood, bone marrow, lymph nodes, and lymphatic system, including acute lymphoblastic leukemia (ALL), chronic lymphocytic lymphoma (CLL), small lymphocytic lymphoma (SLL), acute myelogenous leukemia (AML), chronic myelogenous leukemia (CIVIL), acute monocytic leukemia (AMoL), Hodgkin's lymphoma, and non-Hodgkin's lymphomas. Hematological malignancies are also referred to as “liquid tumors.” Liquid tumor cancers include, but are not limited to, leukemias, myelomas, and lymphomas, as well as other hematological malignancies.

A “solid tumor,” as used herein, refers to an abnormal mass of tissue. Solid tumors may be benign or malignant. Nonlimiting examples of solid tumors include sarcomas, carcinomas, and lymphomas, such as cancers of the lung, breast, prostate, colon, rectum, and bladder. The tissue structure of a solid tumor includes interdependent tissue compartments including the parenchyma (cancer cells) and the supporting stromal cells in which the cancer cells are dispersed, and which may provide a supporting microenvironment.

In some aspects, the cancer is selected from adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In some aspects, the cancer is selected from chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, myxoid/round cell liposarcoma, or telangiectaltic sarcoma. In some aspects, the cancer is selected from acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma. In some aspects, the cancer is selected from acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum. In some aspects, the cancer is selected from Leukemia, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma. In some aspects, the cancer is selected from metastatic melanoma, non-small cell lung cancer, myeloma, esophageal cancer, synovial sarcoma, myxoid/round cell liposarcoma, gastric cancer, breast cancer, hepatocellular cancer, head and neck cancer, ovarian cancer, prostate cancer, bladder cancer, or any combination thereof.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, NKT cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the terms “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

As used herein, the term “anti-tumor immune response” refers to an immune response against a tumor antigen.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject,” “patient,” “individual,” and “host” are used interchangeably herein. As used herein, the phrase “subject in need thereof” includes subjects, such as mammalian subjects, that would benefit, e.g., from administration of immune cells, e.g., modified to express a c-Jun polypeptide and a chimeric binding protein, and cultured using the methods provided herein, as described herein to control tumor growth.

The term “therapeutically effective amount” or “therapeutically effective dosage” refers to an amount of an agent (e.g., an immune cell modified to express a c-Jun polypeptide and a chimeric binding protein, and cultured as described herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations.

The effective amount of the composition (e.g., immune cells modified and cultured as described herein) can, for example, (i) reduce the number of cancer cells; (ii) reduce tumor size; (iii) inhibit, delay, slow to some extent and can stop cancer cell infiltration into peripheral organs; (iv) inhibit (i.e., slow to some extent and can stop tumor metastasis); (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of a composition disclosed herein (e.g., an immune cell modified to express a chimeric binding protein and a c-Jun polypeptide, and cultured as described herein), which is clinically proven to effect a significant decrease in cancer or slowing of progression (regression) of cancer, such as an advanced solid tumor. The ability of a therapeutic agent of the present disclosure (e.g., an immune cell modified and cultured as described herein) to promote disease regression can be evaluated using a variety of methods known to the skilled practitioner, such as in human subjects during clinical trials, in animal model systems predictive of efficacy in humans, or by assaying the activity of the agent in in vitro assays.

The terms “effective” and “effectiveness” with regard to a treatment include both pharmacological effectiveness and physiological safety. Pharmacological effectiveness refers to the ability of a composition disclosed herein (e.g., immune cells modified and cultured as described herein) to promote cancer regression in the patient. Physiological safety refers to the level of toxicity, or other adverse physiological effects at the cellular, organ, and/or organism level (adverse effects) resulting from administration of a composition disclosed herein (e.g., immune cells modified and cultured as described herein).

The terms “chimeric antigen receptor” and “CAR,” as used herein, refer to a set of polypeptides, typically two in the simplest form, which when in an immune effector cell, provides the cell with specificity for a target cell, typically a cancer cell, and with intracellular signal generation. In some aspects, a CAR comprises at least an extracellular antigen-binding domain, a transmembrane domain and a cytoplasmic signaling domain (also referred to herein as “an intracellular signaling domain”) comprising a functional signaling domain derived from a stimulatory molecule and/or costimulatory molecule as defined below. In some aspects, the set of polypeptides are in the same polypeptide chain, e.g., comprise a chimeric fusion protein. In some aspects, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen-binding domain to an intracellular signaling domain. In some aspects, the stimulatory molecule of the CAR is the zeta chain associated with the T cell receptor complex (e.g., CD3 zeta). In some aspects, the cytoplasmic signaling domain comprises a primary signaling domain (e.g., a primary signaling domain of CD3-zeta). In some aspects, the cytoplasmic signaling domain further comprises one or more functional signaling domains derived from at least one costimulatory molecule as defined below. In some aspects, the costimulatory molecule is chosen from the costimulatory molecules described herein, e.g., 4-1BB (i.e., CD137), CD27, and/or CD28.

In some aspects, the CAR comprises a chimeric fusion protein comprising an antigen-binding domain, a transmembrane domain, and an intracellular signaling domain comprising a functional signaling domain derived from a stimulatory molecule, wherein the antigen-binding domain and the transmembrane domain are linked by a CAR spacer. In some aspects, the CAR comprises a chimeric fusion protein comprising an antigen-binding domain linked to a transmembrane domain via a CAR spacer and an intracellular signaling domain comprising a functional signaling domain derived from a costimulatory molecule and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising two functional signaling domains derived from one or more costimulatory molecule(s) and a functional signaling domain derived from a stimulatory molecule. In some aspects, the CAR comprises a chimeric fusion protein comprising an antigen-binding domain linked to a transmembrane domain via a CAR spacer and an intracellular signaling domain comprising at least two functional signaling domains derived from one or more costimulatory molecule(s) and a functional signaling domain derived from a stimulatory molecule. In some aspects, the CAR comprises an optional leader sequence at the amino-terminus (N-terminus) of the CAR. In some aspects, the CAR further comprises a leader sequence at the N-terminus of the antigen-binding domain, wherein the leader sequence is optionally cleaved from the antigen-binding domain (e.g., a scFv) during cellular processing and localization of the CAR to the cellular membrane.

The antigen-specific extracellular domain of a chimeric antigen receptor recognizes and specifically binds an antigen, typically a surface-expressed antigen of a malignancy. An antigen-specific extracellular domain specifically binds an antigen when, for example, it binds the antigen with an affinity constant or affinity of interaction (K_{D}) between about 0.1 pM to about 10 μM, for example, about 0.1 pM to about 1 μM or about 0.1 pM to about 100 nM. Methods for determining the affinity of interaction are known in the art. An antigen-specific extracellular domain suitable for use in a CAR of the present disclosure can be any antigen-binding polypeptide, a wide variety of which are known in the art. In some aspects, the antigen-binding domain is a single chain Fv (scFv). Other antibody-based recognition domains such as cAb VHH (camelid antibody variable domains) and humanized versions thereof, lgNAR VH (shark antibody variable domains) and humanized versions thereof, sdAb VH (single domain antibody variable domains), and “camelized” antibody variable domains are also suitable for use in a CAR of the present disclosure. In some aspects, T cell receptor (TCR) based recognition domains, such as single chain TCR (scTv, i.e., single chain two-domain TCR containing VαVβ) are also suitable for use in the chimeric binding proteins of the present disclosure.

As used herein, the term “T cell receptor” or “TCR” refers to a heterodimer composed of 2 different transmembrane polypeptide chains: an α chain and a β chain, each consisting of a constant region, which anchors the chain inside the T-cell surface membrane, and a variable region, which recognizes and binds to the antigen presented by MHCs. The TCR complex is associated with 6 polypeptides forming 2 heterodimers, CD3γε and CD3δε, and 1 homodimer CD3 ζ, which together forms the CD3 complex. T-cell receptor-engineered T-cell therapy utilizes the modification of T cells that retain these complexes to specifically target the antigens expressed by particular tumor cells. As used herein, the term “TCR” includes naturally occurring TCRs and engineered TCRs.

A “TCR mimic” or a “TCRm” refers to a type of antibody that recognize epitopes comprising both the peptide and the MHC-I molecule, similar to the recognition of such complexes by the TCR on T cells.

The terms “nucleic acids,” “nucleic acid molecules, “nucleotides,” “nucleotide(s) sequence,” and “polynucleotide” can be used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, in some aspects, a polynucleotide of the present disclosure can comprise a single nucleotide sequence encoding a single protein (e.g., codon-optimized c-Jun nucleotide sequence) (“monocistronic”). In some aspects, a polynucleotide of the present disclosure is polycistronic (i.e., comprises two or more cistrons). In some aspects, each of the cistrons of a polycistronic polynucleotide can encode for a protein disclosed herein (e.g., c-Jun protein, chimeric binding protein, or EGFRt). In some aspects, each of the cistrons can be translated independently of one another.

As used herein, the term “polypeptide” encompasses both peptides and proteins, unless indicated otherwise. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

As used herein, the term “fragment” of a polypeptide (e.g., a c-Jun polypeptide) refers to an amino acid sequence of a polypeptide that is shorter than the naturally-occurring sequence, N- and/or C-terminally deleted or any part of the polypeptide deleted in comparison to the naturally occurring polypeptide. Thus, a fragment does not necessary need to have only N- and/or C-terminal amino acids deleted. A polypeptide in which internal amino acids have been deleted with respect to the naturally occurring sequence is also considered a fragment.

As used herein, the term “functional fragment” or “functional portion” refers to a polypeptide fragment that retains polypeptide function. Accordingly, in some aspects, a functional fragment of an Ig hinge, retains the ability to position an antigen-binding domain (e.g., an scFv) in a chimeric binding protein at a distance from a target epitope (e.g., a tumor antigen) such that the antigen-binding domain (e.g., an scFv) can effectively interact with the target epitope (e.g., a tumor antigen). Similarly, in some aspects, a c-Jun functional fragment is a fragment that when expressed in an immune cell (e.g., CAR T cell), results in an immune cell with, e.g., at least about 20%, at least about 25%, at least about 30%, at least about 35%, at least about 40%, at least about 45%, at least about 50%, at least 55%, at least about 60%, at least about 65%, at least about 70%, at least about 75%, at least about 80%, at least about 85%, at least about 90%, at least about 95%, at least about 99%, or about 100% of the activity of a reference immune cell expressing a corresponding full length c-Jun. Non-limiting examples of such activity are further described elsewhere in the present disclosure.

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides encoded by the polynucleotides disclosed herein (e.g., chimeric binding protein and/or c-Jun) can be recombinantly produced using methods known in the art. In some aspects, the polypeptides encoded by the polynucleotides of the present disclosure (e.g., chimeric binding protein and/or c-Jun) are produced by cells, e.g., T cells, following transfection or modification with at least one polynucleotide or vector encoding the polypeptides described here.

As used herein, a “coding region,” “coding sequence,” or “translatable sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′ to 3′),” is complementary to the nucleobase sequence “A-C-T (3′ to 5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence (e.g., miR-485 nucleic acid sequence). Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “expression” as used herein refers to a process by which a polynucleotide produces a gene product, for example, a c-Jun polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

As used herein, the term “identity” refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.” A “reference nucleotide sequence,” when used herein as a comparison to a nucleotide sequence of the disclosure, refers to a polynucleotide sequence essentially identical to the nucleotide sequence of the disclosure except that sequence is not optimized. For example, in some aspects, the reference nucleotide sequence comprises the wild-type JUN nucleic acid sequence.

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In some aspects, the length of a sequence aligned for comparison purposes is at least about 30%, at least about 40%, at least about 50%, at least about 60%, at least about 70%, at least about 80%, at least about 90%, at least about 95%, or about 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In some aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure that has undergone one or more processes of purification. In some aspects, isolating or purifying as used herein is the process of removing, partially removing (e.g., a fraction) of a composition of the present disclosure.

In some aspects, an isolated composition has no detectable undesired activity or, alternatively, the level or amount of the undesired activity is at or below an acceptable level or amount. In some aspects, an isolated composition has an amount and/or concentration of desired composition of the present disclosure, at or above an acceptable amount and/or concentration and/or activity. In some aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

“Administering” (and grammatical variants thereof) refers to the physical introduction of a therapeutic agent (e.g., an engineered cell described herein) to a subject, using any of the various methods and delivery systems known to those skilled in the art. Exemplary routes of administration include intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof. Administering can also be performed, for example, once, a plurality of times, and/or over one or more extended periods.

“Treatment” or “therapy” (including any grammatical derivatives thereof) of a subject refers to any type of intervention or process performed on, or the administration of an active agent to, a subject with the objective of reversing, alleviating, ameliorating, inhibiting, slowing down, or preventing the onset, progression, development, severity, or recurrence of a symptom, complication, condition, or biochemical indicia associated with a disease. In some aspects, the term refers to inducing an immune response in a subject against an antigen.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein the term “therapeutically effective amount” is the amount of reagent or pharmaceutical compound comprising a composition disclosed herein (e.g., modified immune cell described herein) that is sufficient to a produce a desired therapeutic effect, pharmacologic and/or physiologic effect on a subject in need thereof.

A therapeutically effective amount can be a “prophylactically effective amount” as prophylaxis can be considered therapy. As used herein, “prophylactic” refers to a therapeutic or course of action used to prevent the onset of a disease or condition, or to prevent or delay a symptom associated with a disease or condition. As used herein, a “prophylaxis” refers to a measure taken to maintain health and prevent the onset of a disease or condition, or to prevent or delay a symptom associated with a disease or condition.

As used herein, the term “promoter” refers to DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

As used herein, the terms “ug” and “uM” are used interchangeably with “μg” and “μM,” respectively.

Various aspects of the disclosure are described in further detail in the following subsections.

### II. Methods of the Disclosure

Some aspects of the present disclosure provide a method of preparing a population of human immune cells for immunotherapy comprising contacting human immune cells with a programmable cell-signaling scaffold (PCS) in a medium comprising potassium ion at a concentration higher than 5 mM. Some aspects of the present disclosure provide a method of activating a population of human immune cells for immunotherapy comprising contacting human immune cells with a programmable cell-signaling scaffold (PCS) in a medium comprising potassium ion at a concentration higher than 5 mM. Some aspects of the present disclosure provide a method of increasing the yield of activated human immune cells during ex vivo or in vitro culture comprising contacting human immune cells with a programmable cell-signaling scaffold (PCS) in a medium comprising potassium ion at a concentration higher than 5 mM. Some aspects of the present disclosure provide a method of increasing stemness of activated human immune cells while increasing the yield of activated human immune cells during ex vivo or in vitro culture for an immunotherapy comprising contacting human immune cells with a programmable cell-signaling scaffold (PCS) in a medium comprising potassium ion at a concentration higher than 5 mM. Some aspects of the present disclosure provide a method of expanding a population of activated stem-like immune cells ex vivo or in vitro comprising contacting immune cells with programmable cell-signaling scaffolds (PCS) in a medium comprising potassium ion at a concentration higher than 5 mM.

In some aspects, the disclosure is directed to methods of culturing cells, e.g., immune cells, e.g., T cells or NK cell, comprising placing the cells in a metabolic reprogramming medium comprising potassium at a concentration of at least about 5 mM (e.g., higher than 5 mM), wherein the medium is not hypertonic, e.g., hypotonic or isotonic. Some aspects of the present disclosure are directed to methods of culturing cells, e.g., immune cells, e.g., T cells or NK cell, comprising placing the cells in a medium comprising potassium at a concentration higher than 40 mM, e.g., about 50 mM-80 mM. In some aspects, the immune cells comprise T cells, tumor-infiltrating lymphocytes (TILs), natural killer (NK) cells, regulatory T (T_{reg}) cells, or any combination thereof.

Some aspects of the present disclosure are directed to a method of increasing the yield of immune cells, e.g., T cells or NK cell, during ex vivo or in vitro culturing while increasing stemness of the immune cells comprising contacting the immune cells with a programmable cell-signaling scaffold (PCS) in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of preparing a population of immune cells, e.g., T cells or NK cell, for immunotherapy comprising contacting the immune cells with a programmable cell-signaling scaffold (PCS) in a medium comprising potassium ion at a concentration between 40 mM and 80 mM and NaCl at a concentration between 30 mM and 100 mM, wherein the total concentration of potassium ion and NaCl is between 110 and 140 mM. Some aspects of the present disclosure are directed to a method of increasing stemness of immune cells, e.g., T cells or NK cell, during ex vivo or in vitro culturing comprising contacting the immune cells with a programmable cell-signaling scaffold (PCS) in a medium comprising potassium ion at a concentration between 40 mM and 80 mM and NaCl at a concentration between 30 mM and 100 mM, wherein the total concentration of potassium ion and NaCl is between 110 and 140 mM. In some aspects the immune cells are T cells.

In some aspects, the medium is hypotonic. In some aspects, the medium is isotonic. In certain aspects, the medium further comprises interleukin (IL)-2, IL-21, IL-7, IL-15, or any combination thereof. In some aspects, the medium comprises IL-2, IL-7 and IL-15. In some aspects, the medium comprises IL-2 and IL-21. In some aspects, the medium further comprises sodium ion, calcium ion, glucose, or any combination thereof.

II.A. Metabolic Reprogramming Media

Some aspects of the present disclosure are directed to methods of culturing immune cells, e.g., T cells and/or NK cells, comprising contacting immune cells with programmable cell-signaling scaffolds (PCS) in a culture condition (e.g., media), wherein the culture condition (e.g., certain ion concentrations, tonicity of the media, cytokines, and/or any combination thereof) is capable of reducing, limiting or preventing the differentiation of the immune cells, e.g., T cells and/or NK cells, thereby affecting or improving their use in cell therapy, e.g., adoptive cell therapy. In some aspects, the immune cells, e.g., T cells and/or NK cells, are contacted with PCS and cultured in a metabolic reprogramming media (MRM) disclosed herein. In some aspects, the immune cells, e.g., T cells and/or NK cells, contacted with PCS and cultured in MRM have a higher proportion of stem-like cells as compared to cells cultured using conventional methods, e.g., in a medium having less than 5 mM potassium ion and not comprising PCS. In some aspects, the immune cells, e.g., T cells and/or NK cells, contacted with PCS and cultured in MRM have a higher proportion of effector-like cells as compared to cells cultured using conventional methods, e.g., in a medium having less than 5 mM potassium ion. In some aspects, the immune cells, e.g., T cells and/or NK cells, contacted with PCS and cultured in MRM have a higher proportion of both stem-like and effector-like cells as compared to cells cultured using conventional methods, e.g., in a medium having less than 5 mM potassium ion. In some aspects, the immune cells, e.g., T cells and/or NK cells, contacted with PCS and cultured in MRM have a higher proliferative potential as compared to cells cultured using conventional methods, e.g., in a medium having less than 5 mM potassium ion.

Some aspects of the present disclosure are directed to methods of preparing a population of immune cells, e.g., T cells and/or NK cells, comprising contacting the immune cells with PCS in a medium comprising potassium ion at a concentration higher than 5 mM (e.g., a metabolic reprogramming medium disclosed herein). Some aspects of the present disclosure are directed to methods of preparing a population of T cells, comprising contacting T cells with PCS in a medium comprising potassium ion at a concentration higher than 5 mM (e.g., a metabolic reprogramming medium disclosed herein). In some aspects, the present disclosure provides methods of preparing immune cells, e.g., T cells and/or NK cells, comprising contacting the immune cells with PCS in a medium comprising potassium ion at a concentration higher than 5 mM (e.g., higher than 40 mM, e.g., between 55 mM and 70 mM), wherein the method is capable of preserving a stem-like phenotype (e.g., minimal differentiation) of the cultured cells. In some aspects, the present disclosure provides methods of preparing T cells, comprising contacting T cells with PCS in a medium comprising potassium ion at a concentration higher than 5 mM (e.g., higher than 40 mM, e.g., between 55 mM and 70 mM), wherein the method is capable of preserving a stem-like phenotype (e.g., minimal differentiation) of the cultured T cells. In some aspects, the cultured cells have more stem-like phenotypes (e.g., less differentiated) than cells grown in a medium having a lower potassium concentration. In some aspects, the medium further comprises interleukin (IL)-2, IL-21, IL-7, IL-15, or any combination thereof. In some aspects, the medium further comprises sodium ion (e.g., NaCl), calcium ion, glucose, or any combination thereof.

In some aspects, a population of immune cells, e.g., T cells and/or NK cells, cultured using the methods disclosed herein, exhibits an increased number of stem-like cells relative to a population of cells cultured using conventional methods, e.g., in a medium having less than 5 mM potassium ion. In some aspects, a population of T cells, cultured using the methods disclosed herein, exhibits an increased number of stem-like T cells relative to a population of T cells cultured using conventional methods, e.g., in a medium having less than 5 mM potassium ion. In some aspects, the immune cells, e.g., T cells and/or NK cells, exhibit increased expression of markers characteristic of stem-like cells relative to the starting population of immune cells (i.e., prior to the culturing). In some aspects, the T cells, exhibit increased expression of markers characteristic of stem-like cells relative to the starting population of T cells (i.e., prior to the culturing). In some aspects, the starting population of immune cells comprises immune cells (e.g., T cells and/or NK cells) obtained from a subject. In some aspects, the starting population of immune cells comprises T cells obtained from a human subject. In some aspects, the starting population of immune T cells comprises T_{N} cells, T_{SCM} cells, T_{CM} cells, T_{EM} cells, or any combination thereof. In some aspects, the starting population of immune cells comprises T cells prior to transfection/modification with a construct encoding a ligand binding protein as described herein.

Increased cell multipotency can be measured using any methods known in the art. In some aspects, cell stemness is measured by antibody staining followed by gated flow cytometry. In some aspects, the cell stemness is measured by autophagy flux. In some aspects, the cell stemness is measured by glucose uptake. In some aspects, the cell stemness is measured by fatty acid uptake. In some aspects, the cell stemness is measured by mitochondrial biomass. In some aspects, the cell stemness is measured by RNA quantification/expression analysis (e.g., microarray, qPCR (taqman), RNA-Seq., single-cell RNA-Seq., or any combinations thereof). In some aspects, the cell stemness is measured by transcripts that are linked to a metabolism assay (e.g., a seahorse metabolism assay, analysis of extracellular acidification rate (ECAR); analysis of oxygen consumption rate (OCR); analysis of spare respiratory capacity; and/or analysis of mitochondrial membrane potential). In some aspects, stemness is measured using one or more in vivo or in vitro functional assays (e.g., assaying cell persistence, antitumor capacity, antitumor clearance, viral clearance, multipotency, cytokine release, cell killing, or any combination thereof).

In some aspects, the differentiation status of the immune cells, e.g., T cells and/or NK cells, is characterized by increased numbers of cells expressing markers typical of less differentiated cells. In some aspects, the differentiation status of the T cells is characterized by increased numbers of cells expressing markers typical of less differentiated T cells. In some aspects, an increase in the number of stem-like cells is characterized by increased numbers of T cells expressing markers typical of T_{N} and/or T_{SCM} cells. In some aspects, an increase in the number of stem-like T cells is characterized by increased numbers of cells expressing markers typical of T_{SCM} cells. In some aspects, the T cell population exhibits an increased number of cells that express CD45RA. In some aspects, the T cell population exhibits an increased number of cells that express CCR7. In some aspects, the T cell population exhibits an increased number of cells that express CD62L. In some aspects, the T cell population exhibits an increased number of cells that express CD28. In some aspects, the T cell population exhibits an increased number of cells that express CD95. In some aspects, the cells are CD45RO^{low}. In some aspects, the cells do not express CD45RO. In some aspects, the cell population exhibits an increased number of cells that are CD45RA⁺, CCR7⁺, and CD62L⁺. In some aspects, the cell population exhibits an increased number of cells that are CD95⁺, CD45RA⁺, CCR7⁺, and CD62L⁺. In some aspects, the cell population exhibits an increased number of cells that express TCF7. In some aspects, the T cell population exhibits an increased number of cells that are CD45RA⁺, CCR7⁺, CD62L⁺, and TCF7⁺. In some aspects, the T cell population exhibits an increased number of cells that are CD95⁺, CD45RA, CCR7⁺, CD62L⁺, and TCF7⁺. In some aspects, the T cell population exhibits an increased number of cells that are CD3⁺, CD45RA⁺, CCR7⁺, CD62L⁺, and TCF7⁺. In some aspects, the T cell population exhibits an increased number of cells that are CD3⁺, CD95⁺, CD45RA⁺, CCR7⁺, CD62L⁺, and TCF7⁺. In some aspects, the cells express CD27. In some aspects, the T cell population exhibits an increased number of cells that are CD27⁺, CD3⁺, CD45RA⁺, CCR7⁺, CD62L⁺, and TCF7⁺. In some aspects, the T cell population exhibits an increased number of cells that are CD27⁺, CD3⁺, CD95⁺, CD45RA⁺, CCR7⁺, CD62L⁺, and TCF7⁺. In some aspects, the T cell population exhibits an increased number of cells that are CD39⁻ and CD69⁻. In some aspects the T cell population exhibits an increased number of cells that are TCF7⁻ and CD39⁻. In some aspects, the cell population exhibits an increased number of T_{SCM} cells. In some aspects, the cell population exhibits an increased number of T_{N} cells. In some aspects, the cell population exhibits an increased number of T_{SCM} and T_{N} cells. In some aspects, the cell population exhibits an increased number of stem-like T cells. In some aspects the T cells are CD4+ cells; in some aspects the T cells are CD8+ cells.

In some aspects, the number of stem-like cells in the culture is increased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%, relative to the number of stem-like cells prior to culture with MRM. In some aspects, the number of stem-like cells in the culture is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, or at least about 20-fold, relative to the number of stem-like cells prior to culture with MRM.

In some aspects, following culture of T cells according to the methods disclosed herein, stem-like T cells constitute at least about 1%, at least about 2%, at least about 3%, at least about 4%, at least about 5%, at least about 10%, or at least about 15% of the total number of CD8⁺ T cells in the culture. In some aspects, following culture of T cells according to the methods disclosed herein, stem-like T cells constitute at least about 1%, at least about 2%, at least about 3%, at least about 4%, at least about 5%, at least about 10%, or at least about 15% of the total number of CD4⁺ T cells in the culture.

In some aspects, following culture of T cells according to the methods disclosed herein, stem-like T cells constitute at least about 10% to at least about 70% of the total number of T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD8⁺ T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD4⁺ T cells in the culture.

In some aspects, following culture of T cells according to the methods disclosed herein, at least about 10% to at least about 40% of the total number of T cells in the culture are CD39⁻/CD69⁻ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/CD69⁻ T cells.

In some aspects, following culture of T cells according to the methods disclosed herein, at least about 10% to at least about 70% of the total number of T cells in the culture are CD39⁻/TCF7⁺ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/TCF7⁺ T cells. In some aspects the T cells are CD4⁺ T cells. In some aspects the T cells are CD8⁺ T cells.

In some aspects, the immune cells, e.g., engineered immune cells (e.g., T cells and/or NK cells) of the present disclosure, cultured according to the methods disclosed herein, exhibit increased transduction efficiency. In some aspects, the engineered T cells cultured according to the methods disclosed herein, exhibit increased transduction efficiency. In some aspects, a greater percentage of cells express a target transgene, e.g., encoding a ligand binding protein, following transduction, wherein the cells are cultured according to the methods disclosed herein as compared to cells similarly transduced and cultured using conventional methods, (e.g., in media containing less than 5 mM K⁺). In certain aspects, a greater percentage of cells cultured according to the methods disclosed herein express a ligand binding protein following lentiviral transduction of the cells, as compared to similarly transduced cells cultured using conventional methods, e.g., in media containing less than 5 mM K⁺. In some aspects, transduction efficiency is increased at least about 1.5-fold relative to similarly transduced cells cultured using conventional methods, e.g., in media containing less than 5 mM K⁺. In some aspects, transduction efficiency is increase at least about 2-fold relative to similarly transduced cells cultured using conventional methods, e.g., in media containing less than 5 mM K⁺.

In some aspects, the immune cells, e.g., T cells and/or NK cells, are transduced before culturing according to the methods disclosed herein. In some aspects, the immune cells, e.g., T cells and/or NK cells, are transduced after culturing according to the methods disclosed herein. In some aspects, the immune cells, e.g., T cells and/or NK cells, are cultured according to the methods disclosed herein, e.g., by contacting the immune cells with an APC-MS in a medium comprising at least 5 mM potassium ion, prior to, during, and after transduction.

In certain aspects, the immune cells are transduced using a viral vector. In some aspects, the vector comprises a lentiviral vector, adenoviral vector, adeno-associated viral vector, vaccinia vector, herpes simplex viral vector, and Epstein-Barr viral vector. In some aspects, the viral vector comprises a retrovirus. In some aspects, the viral vector comprises a lentivirus. In some aspects, the viral vector comprises an AAV.

In some aspects, the immune cells are transduced using a non-viral method. In some aspects, the non-viral method includes the use of a transposon. In some aspects, use of a non-viral method of delivery permits reprogramming of immune cells, e.g., T cells and/or NK cells, and direct infusion of the cells into the subject. In some aspects, the polynucleotide can be inserted into the genome of a target cell (e.g., a T cell) or a host cell (e.g., a cell for recombinant expression of the encoded proteins) by using CRISPR/Cas systems and genome edition alternatives such as zinc-finger nucleases (ZFNs), transcription activator-like effector nucleases (TALENs), and meganucleases (MNs).

In some aspects, upon adoptive transfer of the immune cells, e.g., T cells and/or NK cells, optionally expressing a ligand binding protein, cultured according to the methods disclosed herein, the transferred cells exhibit decreased cell exhaustion, as compared to cells cultured using conventional methods, e.g., in media containing less than 5 mM K⁺. In some aspects, upon adoptive transfer of the T cells, optionally expressing a ligand binding protein, cultured according to the methods disclosed herein, the transferred T cells exhibit decreased cell exhaustion, as compared to T cells cultured using conventional methods, e.g., in media containing less than 5 mM K⁺. In some aspects, upon adoptive transfer of the cells cultured according to the methods disclosed herein, the transferred cells persist for a longer period of time in vivo, as compared to cells cultured using conventional methods, e.g., in media containing less than 5 mM K⁺. In some aspects, the transferred cells, e.g., T cells and/or NK cells, have a greater in vivo efficacy, e.g., tumor-killing activity, as compared to cells cultured using conventional methods, e.g., in media containing less than 5 mM K⁺. In some aspects, a lower dose of the cells cultured according to the methods disclosed herein is needed to elicit a response, e.g., decreased tumor volume, in a subject as compared to cells cultured using conventional methods, e.g., in media containing less than 5 mM K⁺.

In some aspects, the immune cells (e.g., T cells and/or NK cells) are cultured according to the methods disclosed herein, e.g., by contacting the immune cells with PCS in a medium comprising at least 5 mM potassium ion, immediately upon isolation from a subject. In some aspects, the immune cells, e.g., T cells and/or NK cells, are cultured according to the methods disclosed herein during expansion of the cells. In some aspects, the immune cells, e.g., T cells and/or NK cells, are cultured according to the methods disclosed herein during engineering of the cells, e.g., during transduction with a construct encoding a transgene, e.g., a ligand binding protein. In some aspects, the immune cells, e.g., T cells and/or NK cells, are cultured according to the methods disclosed herein following engineering of the cells, e.g., following transduction with a construct encoding a transgene, e.g., a ligand binding protein. In some aspects, the immune cells, e.g., T cells and/or NK cells, are cultured according to the methods disclosed herein throughout expansion and engineering. In some aspects, the immune cells, e.g., T cells and/or NK cells, are cultured according to the methods disclosed herein throughout viral genetic engineering. In some aspects, the immune cells, e.g., T cells and/or NK cells, are cultured according to the methods disclosed herein throughout non-viral genetic engineering. In some aspects, the immune cells, e.g., T cells and/or NK cells, are cultured according to the methods disclosed herein during introduction of ligand binding proteins to the immune cell (e.g., T cells and/or NK cells) to allow for tumor specific targeting (e.g., a CAR, TCR, or a TCR mimic). In some aspects, the immune cells, e.g., T cells and/or NK cells, are cultured according to the methods disclosed herein throughout introduction of one or more endogenous genes that improve T cell function. In some aspects, the immune cells, e.g., T cells and/or NK cells, are cultured according to the methods disclosed herein throughout introduction of one or more synthetic genes that improve T cell function.

In some aspects, the immune cells, e.g., T cells and/or NK cells, are cultured according to the methods disclosed herein, e.g., by contacting the immune cells with PCS in a medium comprising at least 5 mM potassium ion, e.g., from the time the immune cells, e.g., T cells and/or NK cells, are isolated from a subject, through growing, expansion, engineering, and until administration into a subject in need of adoptive cell therapy. In some aspects, the T cells are cultured according to the methods disclosed herein, e.g., by contacting the immune cells with PCS in a medium comprising at least 5 mM potassium ion, for the entirety of ex vivo culture, e.g., from the time the T cells are isolated from a subject, through growing, expansion, engineering, and until administration into a subject in need of adoptive cell therapy. In some aspects, the immune cells, e.g., T cells and/or NK cells, are cultured according to the methods disclosed herein, e.g., by contacting the immune cells with PCS in a medium comprising at least 5 mM potassium ion, for the duration of expansion. In some aspects, the immune cells, e.g., T cells and/or NK cells, are cultured according to the methods disclosed herein, e.g., by contacting the immune cells with PCS in a medium comprising at least 5 mM potassium ion, until the total number of viable immune cells, e.g., T cells and/or NK cells, is at least about 10⁴, at least about 5×10⁴, at least about 10⁵, at least about 5×10⁵, at least about 10⁶, or at least about 5×10⁶, at least about 1×10⁷, at least about 5×10⁷, at least about 1×10⁸, at least about 5×10⁸, at least about 1×10⁹, at least about 5×10⁹, at least about 1×10¹⁰, at least about 5×10¹⁰, at least about 1×10¹¹, at least about 5×10¹¹, at least about 1×10¹², or at least about 5×10¹² total cells. In some aspects, the T cells are cultured according to the methods disclosed herein until the total number of viable T cells is at least about 10⁴, at least about 5×10⁴, at least about 10⁵, at least about 5×10⁵, at least about 10⁶, or at least about 5×10⁶, at least about 1×10⁷, at least about 5×10⁷, at least about 1×10⁸, at least about 5×10⁸, at least about 1×10⁹, at least about 5×10⁹, at least about 1×10¹⁰, at least about 5×10¹⁰, at least about 1×10¹¹, at least about 5×10¹¹, at least about 1×10¹², or at least about 5×10¹² total T cells.

In some aspects, the medium further comprises a cell expansion agent. As used herein, a “cell expansion agent” refers to an agent, e.g., small molecule, polypeptide, or any combination thereof, that promotes the in vitro and/or ex vivo growth and proliferation of cultured cells, e.g., immune cells (e.g., T cells and/or NK cells). In some aspects, the cell expansion agent comprises a PI3K inhibitor. In some aspects, the medium further comprises an AKT inhibitor. In some aspects, the medium further comprises a PI3K inhibitor and an AKT inhibitor. In some aspects, the PI3K inhibitor comprises LY294002. In some aspects, the PI3K inhibitor comprises IC87114. In some aspects, the PI3K inhibitor comprises idelalisib (see, e.g., Peterson et al., Blood Adv. 2(3):210-23 (2018)). In some aspects, the medium further comprises a GSK3B inhibitor. In some aspects, the GSK3B inhibitor comprises TWS119. In some aspects, the medium further comprises an ACLY inhibitor. In some aspects, the ACLY inhibitor comprises potassium hydroxycitrate tribasic monohydrate. In some aspects, the PI3K inhibitor comprises hydroxyl citrate. In some aspects, the PI3K inhibitor comprises pictilisib. In some aspects, the PI3K inhibitor comprises CAL-101. In some aspects, the AKT inhibitor comprises MK2206, A443654, or AKTi-VIII (CAS 612847-09-3). In some aspects, the cell expansion agent is linked to or associated with the PCS.

In some aspects, the metabolic reprogramming media comprises a mitochondrial fuel. In some aspects, the metabolic reprogramming media comprises O-Acetyl-L-carnitine hydrochloride. In some aspects, the metabolic reprogramming media comprises at least about 0.1 mM, at least about 0.5 mM, at least about 1.0 mM, at least about 5 mM, or at least about 10 mM O-Acetyl-L-carnitine hydrochloride. In some aspects, the metabolic reprogramming media comprises at least about 1.0 mM O-Acetyl-L-carnitine hydrochloride. In some aspects, the mitochondrial fuel is linked to or associated with the PCS.

In some aspects, the metabolic reprogramming media further comprises one or more of (i) one or more cell expansion agents, (ii) sodium ion (e.g., NaCl), (iii) one or more saccharides, (iv) calcium ion, and (v) one or more cytokines.

II.A.1. Potassium

Some aspects of the disclosure are directed to methods of culturing immune cells, e.g., T cells and/or NK cells, comprising contacting the immune cells with PCS in a medium comprising an increased concentration of potassium ion (e.g., greater than about 5 mM, greater than about 40 mM, greater than about 45 mM, greater than about 50 mM, greater than about 55 mM, greater than about 60 mM, greater than about 65 mM, or greater than about 70 mM), i.e., a metabolic reprogramming medium disclosed herein, relative to a control medium. In some aspects, the metabolic reprogramming medium comprises at least about 5 mM to at least about 100 mM potassium ion, at least about 5 mM to at least about 90 mM potassium ion, at least about 5 mM to at least about 80 mM potassium ion, at least about 5 mM to at least about 75 mM potassium ion, at least about 5 mM to at least about 70 mM potassium ion, at least about 5 mM to at least about 65 mM potassium ion, at least about 5 mM to at least about 60 mM potassium ion, at least about 5 mM to at least about 55 mM potassium ion, at least about 5 mM to at least about 50 mM potassium ion, at least about 5 mM to at least about 45 mM potassium ion, at least about 5 mM to at least about 40 mM potassium ion, at least about 10 mM to at least about 80 mM potassium ion, at least about 10 mM to at least about 75 mM potassium ion, at least about 10 mM to at least about 70 mM potassium ion, at least about 10 mM to at least about 65 mM potassium ion, at least about 10 mM to at least about 60 mM potassium ion, at least about 10 mM to at least about 55 mM potassium ion, at least about 10 mM to at least about 50 mM potassium ion, at least about 10 mM to at least about 45 mM potassium ion, at least about 10 mM to at least about 40 mM potassium ion, at least about 20 mM to at least about 80 mM potassium ion, at least about 20 mM to at least about 75 mM potassium ion, at least about 20 mM to at least about 70 mM potassium ion, at least about 20 mM to at least about 65 mM potassium ion, at least about 20 mM to at least about 60 mM potassium ion, at least about 20 mM to at least about 55 mM potassium ion, at least about 20 mM to at least about 50 mM potassium ion, at least about 20 mM to at least about 45 mM potassium ion, at least about 20 mM to at least about 40 mM potassium ion, at least about 30 mM to at least about 80 mM potassium ion, at least about 30 mM to at least about 75 mM potassium ion, at least about 30 mM to at least about 70 mM potassium ion, at least about 30 mM to at least about 65 mM potassium ion, at least about 30 mM to at least about 60 mM potassium ion, at least about 30 mM to at least about 55 mM potassium ion, at least about 30 mM to at least about 50 mM potassium ion, at least about 30 mM to at least about 45 mM potassium ion, at least about 30 mM to at least about 40 mM potassium ion, at least about 40 mM to at least about 80 mM potassium ion, at least about 40 mM to at least about 75 mM potassium ion, at least about 40 mM to at least about 70 mM potassium ion, at least about 40 mM to at least about 65 mM potassium ion, at least about 40 mM to at least about 60 mM potassium ion, at least about 40 mM to at least about 55 mM potassium ion, at least about 40 mM to at least about 50 mM potassium ion, at least about 40 mM to at least about 45 mM potassium ion, at least about 45 mM to at least about 80 mM potassium ion, at least about 45 mM to at least about 75 mM potassium ion, at least about 45 mM to at least about 70 mM potassium ion, at least about 45 mM to at least about 65 mM potassium ion, at least about 45 mM to at least about 60 mM potassium ion, at least about 45 mM to at least about 55 mM potassium ion, at least about 45 mM to at least about 50 mM potassium ion, at least about 50 mM to at least about 80 mM potassium ion, at least about 50 mM to at least about 75 mM potassium ion, at least about 50 mM to at least about 70 mM potassium ion, at least about 50 mM to at least about 65 mM potassium ion, at least about 50 mM to at least about 60 mM potassium ion, or at least about 50 mM to at least about 55 mM potassium ion.

In some aspects, the metabolic reprogramming medium comprises at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 55 mM, at least about 60 mM, at least about 65 mM, at least about 70 mM, at least about 75 mM, or at least about 80 mM potassium ion. In some aspects, the metabolic reprogramming medium comprises at least about 5 mM potassium ion. In some aspects, the metabolic reprogramming medium comprises at least about 10 mM potassium ion. In some aspects, the metabolic reprogramming medium comprises at least about 15 mM potassium ion. In some aspects, the metabolic reprogramming medium comprises at least about 20 mM potassium ion. In some aspects, the metabolic reprogramming medium comprises at least about 25 mM potassium ion. In some aspects, the metabolic reprogramming medium comprises at least about 30 mM potassium ion. In some aspects, the metabolic reprogramming medium comprises at least about 35 mM potassium ion. In some aspects, the metabolic reprogramming medium comprises at least about 40 mM potassium ion. In some aspects, the metabolic reprogramming medium comprises at least about 45 mM potassium ion. In some aspects, the metabolic reprogramming medium comprises at least about 50 mM potassium ion. In some aspects, the metabolic reprogramming medium comprises at least about 55 mM potassium ion. In some aspects, the metabolic reprogramming medium comprises at least about 60 mM potassium ion. In some aspects, the metabolic reprogramming medium comprises at least about 65 mM potassium ion. In some aspects, the metabolic reprogramming medium comprises at least about 70 mM potassium ion. In some aspects, the metabolic reprogramming medium comprises at least about 75 mM potassium ion. In some aspects, the metabolic reprogramming medium comprises at least about 80 mM potassium ion.

In some aspects, the metabolic reprogramming medium comprises an increased concentration of potassium ion, e.g., at least about 5 mM potassium ion, and the medium is hypotonic. In some aspects, the metabolic reprogramming medium comprises potassium ion at a concentration between about 40 mM and about 80 mM and NaCl at a concentration between about 30 mM and about 100 mM, wherein the total concentration of potassium ion and NaCl is between about 110 and about 140 mM.

In some aspects, the concentration of potassium ion in a metabolic reprogramming medium of the present disclosure is about 5 mM to about 100 mM. In some aspects, the concentration of potassium ion in a metabolic reprogramming medium of the present disclosure is about 5 mM to about 100 mM, wherein the medium is hypotonic. In some aspects, the concentration of potassium ion in a metabolic reprogramming medium of the present disclosure is about 5 mM to about 90 mM, about 5 mM to about 80 mM, about 5 mM to about 70 mM, about 5 mM to about 60 mM, or about 5 mM to about 50 mM. In some aspects, the concentration of potassium ion in a metabolic reprogramming medium of the present disclosure is about 5 mM to about 90 mM, about 5 mM to about 80 mM, about 5 mM to about 70 mM, about 5 mM to about 60 mM, or about 5 mM to about 50 mM, wherein the medium is hypotonic. In some aspects, the concentration of potassium ion in a metabolic reprogramming medium of the present disclosure is about 25 mM to about 100 mM. In some aspects, the concentration of potassium ion in a metabolic reprogramming medium of the present disclosure is about 25 mM to about 100 mM, wherein the medium is hypotonic. In some aspects, the concentration of potassium ion in a metabolic reprogramming medium of the present disclosure is about 25 mM to about 90 mM, about 25 mM to about 80 mM, about 25 mM to about 70 mM, about 25 mM to about 60 mM, or about 25 mM to about 50 mM. In some aspects, the concentration of potassium ion in a metabolic reprogramming medium of the present disclosure is about 25 mM to about 90 mM, about 25 mM to about 80 mM, about 25 mM to about 70 mM, about 25 mM to about 60 mM, or about 25 mM to about 50 mM, wherein the medium is hypotonic. In some aspects, the concentration of potassium ion in a metabolic reprogramming medium of the present disclosure is about 40 mM to about 100 mM. In some aspects, the concentration of potassium ion in a metabolic reprogramming medium of the present disclosure is about 40 mM to about 100 mM, wherein the medium is hypotonic. In some aspects, the concentration of potassium ion is about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM. In some aspects, the concentration of potassium ion is about 40 mM to about 90 mM, about 40 mM to about 85 mM, about 40 mM to about 80 mM, about 40 mM to about 75 mM, about 40 mM to about 70 mM, about 40 mM to about 65 mM, about 40 mM to about 60 mM, about 40 mM to about 55 mM, or about 40 mM to about 50 mM, wherein the medium is hypotonic. In some aspects, the concentration of potassium ion is about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM. In some aspects, the concentration of potassium ion is about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM, and wherein the medium is hypotonic. In some aspects, the metabolic reprogramming medium comprises at least about 50 mM potassium ion and less than about 90 mM NaCl. In some aspects, the total concentration of potassium ion and NaCl is between 110 mM and 140 mM.

In some aspects, the concentration of potassium ion in a metabolic reprogramming medium of the present disclosure is about 50 mM to about 120 mM. In some aspects, the concentration of potassium ion is about 50 mM to about 115 mM, about 50 mM to about 110 mM, about 50 mM to about 105 mM, about 50 mM to about 100 mM, about 50 mM to about 95 mM, about 50 mM to about 90 mM, about 50 mM to about 85 mM, about 50 mM to about 80 mM, about 50 mM to about 75 mM, about 50 mM to about 70 mM, about 50 mM to about 65 mM, about 50 mM to about 60 mM, or about 50 mM to about 55 mM. In some aspects, the medium is hypotonic. In some aspects, the medium comprises at least about 50 mM to about 120 mM potassium ion and less than about 90 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 55 mM to about 120 mM. In some aspects, the concentration of potassium ion is about 55 mM to about 115 mM, about 55 mM to about 110 mM, about 55 mM to about 105 mM, about 55 mM to about 100 mM, about 55 mM to about 95 mM, about 55 mM to about 90 mM, about 55 mM to about 85 mM, about 55 mM to about 80 mM, about 55 mM to about 75 mM, about 55 mM to about 70 mM, about 55 mM to about 65 mM, or about 55 mM to about 60 mM. In some aspects, the medium is hypotonic. In some aspects, the metabolic reprogramming medium comprises at least about 55 mM to about 120 mM potassium ion and less than about 85 mM to about 20 mM NaCl. In some aspects, the total concentration of potassium ion and NaCl in a metabolic reprogramming medium of the present disclosure is between 110 mM and 140 mM.

In some aspects, the concentration of potassium ion in a metabolic reprogramming medium of the present disclosure is about 60 mM to about 120 mM. In some aspects, the concentration of potassium ion is about 60 mM to about 115 mM, about 60 mM to about 110 mM, about 60 mM to about 105 mM, about 60 mM to about 100 mM, about 60 mM to about 95 mM, about 60 mM to about 90 mM, about 60 mM to about 85 mM, about 60 mM to about 80 mM, about 60 mM to about 75 mM, about 60 mM to about 70 mM, or about 60 mM to about 65 mM. In some aspects, the medium is hypotonic. In some aspects, the metabolic reprogramming medium comprises at least about 60 mM to about 120 mM potassium ion and less than about 80 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 65 mM to about 120 mM. In some aspects, the concentration of potassium ion is about 65 mM to about 115 mM, about 65 mM to about 110 mM, about 65 mM to about 105 mM, about 65 mM to about 100 mM, about 65 mM to about 95 mM, about 65 mM to about 90 mM, about 65 mM to about 85 mM, about 65 mM to about 80 mM, about 65 mM to about 75 mM, or about 65 mM to about 70 mM. In some aspects, the medium is hypotonic. In some aspects, the metabolic reprogramming medium comprises at least about 65 mM to about 120 mM potassium ion and less than about 75 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 70 mM to about 120 mM. In some aspects, the concentration of potassium ion is about 70 mM to about 115 mM, about 70 mM to about 110 mM, about 70 mM to about 105 mM, about 70 mM to about 100 mM, about 70 mM to about 95 mM, about 70 mM to about 90 mM, about 70 mM to about 85 mM, about 70 mM to about 80 mM, or about 70 mM to about 75 mM. In some aspects, the medium is hypotonic. In some aspects, the metabolic reprogramming medium comprises at least about 70 mM to about 120 mM potassium ion and less than about 70 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 75 mM to about 120 mM. In some aspects, the concentration of potassium ion is about 75 mM to about 115 mM, about 75 mM to about 110 mM, about 75 mM to about 105 mM, about 75 mM to about 100 mM, about 75 mM to about 95 mM, about 75 mM to about 90 mM, about 75 mM to about 85 mM, or about 75 mM to about 80 mM. In some aspects, the medium is hypotonic. In some aspects, the metabolic reprogramming medium comprises at least about 75 mM to about 120 mM potassium ion and less than about 65 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 80 mM to about 120 mM. In some aspects, the concentration of potassium ion is about 80 mM to about 115 mM, about 80 mM to about 110 mM, about 80 mM to about 105 mM, about 80 mM to about 100 mM, about 80 mM to about 95 mM, about 80 mM to about 90 mM, or about 80 mM to about 85 mM. In some aspects, the medium is hypotonic. In some aspects, the metabolic reprogramming medium comprises at least about 80 mM to about 120 mM potassium ion and less than about 60 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 85 mM to about 120 mM. In some aspects, the concentration of potassium ion is about 85 mM to about 115 mM, about 85 mM to about 110 mM, about 85 mM to about 105 mM, about 85 mM to about 100 mM, about 85 mM to about 95 mM, or about 85 mM to about 90 mM. In some aspects, the medium is hypotonic. In some aspects, the metabolic reprogramming medium comprises at least about 85 mM to about 120 mM potassium ion and less than about 65 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 90 mM to about 120 mM. In some aspects, the concentration of potassium ion is about 90 mM to about 115 mM, about 90 mM to about 110 mM, about 90 mM to about 105 mM, about 90 mM to about 100 mM, or about 90 mM to about 95 mM. In some aspects, the medium is hypotonic. In some aspects, the metabolic reprogramming medium comprises at least about 90 mM to about 120 mM potassium ion and less than about 50 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 95 mM to about 120 mM. In some aspects, the concentration of potassium ion is about 95 mM to about 115 mM, about 95 mM to about 110 mM, about 95 mM to about 105 mM, or about 95 mM to about 100 mM. In some aspects, the medium is hypotonic. In some aspects, the metabolic reprogramming medium comprises at least about 95 mM to about 120 mM potassium ion and less than about 55 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 100 mM to about 120 mM. In some aspects, the concentration of potassium ion is about 100 mM to about 115 mM, about 100 mM to about 110 mM, or about 100 mM to about 105 mM. In some aspects, the medium is hypotonic. In some aspects, the metabolic reprogramming medium comprises at least about 100 mM to about 120 mM potassium ion and less than about 50 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 105 mM to about 120 mM. In some aspects, the concentration of potassium ion is about 105 mM to about 115 mM, or about 105 mM to about 110 mM. In some aspects, the medium is hypotonic. In some aspects, the metabolic reprogramming medium comprises at least about 105 mM to about 120 mM potassium ion and less than about 35 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 110 mM to about 120 mM. In some aspects, the concentration of potassium ion is about 110 mM to about 115 mM. In some aspects, the medium is hypotonic. In some aspects, the metabolic reprogramming medium comprises at least about 110 mM to about 120 mM potassium ion and less than about 30 mM to about 20 mM NaCl. In some aspects, the total concentration of potassium ion and NaCl is between 110 mM and 140 mM.

In some aspects, the concentration of potassium ion in a metabolic reprogramming medium of the present disclosure is about 50 mM to about 90 mM. In some aspects, the concentration of potassium ion is about 50 mM to about 80 mM. In some aspects, the concentration of potassium ion is about 60 mM to about 90 mM. In some aspects, the concentration of potassium ion is about 60 mM to about 80 mM. In some aspects, the concentration of potassium ion is about 70 mM to about 90 mM. In some aspects, the concentration of potassium ion is about 70 mM to about 80 mM. In some aspects, the concentration of potassium ion is about 80 mM to about 90 mM.

In some aspects, the concentration of potassium ion in a metabolic reprogramming medium of the present disclosure is about 50 mM to about 90 mM, and the concentration of NaCl is less than about 90 mM to about 50 mM. In some aspects, the concentration of potassium ion is about 50 mM to about 80 mM, and the concentration of NaCl is less than about 90 mM to about 60 mM. In some aspects, the concentration of potassium ion is about 60 mM to about 90 mM, and the concentration of NaCl is less than about 90 mM to about 60 mM. In some aspects, the concentration of potassium ion is about 60 mM to about 80 mM, and the concentration of NaCl is less than about 80 mM to about 60 mM. In some aspects, the concentration of potassium ion is about 70 mM to about 90 mM, and the concentration of NaCl is less than about 70 mM to about 50 mM. In some aspects, the concentration of potassium ion is about 70 mM to about 80 mM, and the concentration of NaCl is less than about 70 mM to about 60 mM. In some aspects, the concentration of potassium ion is about 80 mM to about 90 mM, and the concentration of NaCl is less than about 60 mM to about 50 mM. In some aspects, the total concentration of potassium ion and NaCl is between 110 mM and 140 mM.

In some aspects, the concentration of potassium ion in a metabolic reprogramming medium of the present disclosure is about 50 mM to about 55 mM. In some aspects, the concentration of potassium ion is about 50 mM to about 55 mM, and the concentration of NaCl is less than about 90 to about 85. In some aspects, the concentration of potassium ion is about 55 mM to about 60 mM. In some aspects, the concentration of potassium ion is about 55 mM to about 60 mM, and the concentration of NaCl is less than about 85 to about 80. In some aspects, the concentration of potassium ion is about 60 mM to about 65 mM. In some aspects, the concentration of potassium ion is about 60 mM to about 65 mM, and the concentration of NaCl is less than about 80 mM to about 75 mM. In some aspects, the concentration of potassium ion is about 65 mM to about 70 mM. In some aspects, the concentration of potassium ion is about 65 mM to about 70 mM, and the concentration of NaCl is less than about 75 mM to about 70 mM. In some aspects, the concentration of potassium ion is about 70 mM to about 75 mM. In some aspects, the concentration of potassium ion is about 70 mM to about 75 mM, and the concentration of NaCl is less than about 70 mM to about 65 mM. In some aspects, the concentration of potassium ion is about 75 mM to about 80 mM. In some aspects, the concentration of potassium ion is about 75 mM to about 80 mM, and the concentration of NaCl is less than about 65 mM to about 60 mM. In some aspects, the concentration of potassium ion is about 80 mM to about 85 mM. In some aspects, the concentration of potassium ion is about 80 mM to about 85 mM, and the concentration of NaCl is less than about 60 mM to about 55 mM. In some aspects, the concentration of potassium ion is about 85 mM to about 90 mM. In some aspects, the concentration of potassium ion is about 85 mM to about 90 mM, and the concentration of NaCl is less than about 55 mM to about 50 mM. In some aspects, the concentration of potassium ion is about 90 mM to about 95 mM. In some aspects, the concentration of potassium ion is about 90 mM to about 95 mM, and the concentration of NaCl is less than about 50 to about 45. In some aspects, the concentration of potassium ion is about 95 mM to about 100 mM. In some aspects, the concentration of potassium ion is about 95 mM to about 100 mM, and the concentration of NaCl is less than about 45 mM to about 40 mM. In some aspects, the concentration of potassium ion is about 100 mM to about 105 mM. In some aspects, the concentration of potassium ion is about 100 mM to about 105 mM, and the concentration of NaCl is less than about 40 mM to about 35 mM. In some aspects, the concentration of potassium ion is about 105 mM to about 110 mM. In some aspects, the concentration of potassium ion is about 105 mM to about 110 mM, and the concentration of NaCl is less than about 35 to about 30. In some aspects, the concentration of potassium ion is about 110 mM to about 115 mM. In some aspects, the concentration of potassium ion is about 110 mM to about 115 mM, and the concentration of NaCl is less than about 30 mM to about 25 mM. In some aspects, the concentration of potassium ion is about 115 mM to about 120 mM. In some aspects, the concentration of potassium ion is about 115 mM to about 120 mM, and the concentration of NaCl is less than about 25 mM to about 20 mM. In some aspects, the total concentration of potassium ion and NaCl is between 110 mM and 140 mM.

In some aspects, the concentration of potassium ion is about 40 mM to about 90 mM, wherein the medium is hypotonic or isotonic. In some aspects, the concentration of potassium ion is about 40 mM to about 80 mM, wherein the medium is hypotonic or isotonic. In some aspects, the concentration of potassium ion is about 40 mM to about 70 mM, wherein the medium is hypotonic or isotonic. In some aspects, the concentration of potassium ion is about 50 mM to about 90 mM, wherein the medium is hypotonic or isotonic. In some aspects, the concentration of potassium ion is about 50 mM to about 80 mM, wherein the medium is hypotonic or isotonic. In some aspects, the concentration of potassium ion is about 50 mM to about 70 mM, wherein the medium is hypotonic or isotonic. In some aspects, the concentration of potassium ion is about 55 mM to about 90 mM, wherein the medium is hypotonic or isotonic. In some aspects, the concentration of potassium ion is about 55 mM to about 80 mM, wherein the medium is hypotonic or isotonic. In some aspects, the concentration of potassium ion is about 55 mM to about 70 mM, wherein the medium is hypotonic or isotonic. In some aspects, the concentration of potassium ion is about 60 mM to about 90 mM, wherein the medium is hypotonic or isotonic. In some aspects, the concentration of potassium ion is about 60 mM to about 80 mM, wherein the medium is hypotonic or isotonic. In some aspects, the concentration of potassium ion is about 60 mM to about 70 mM, wherein the medium is hypotonic or isotonic. In some aspects, the concentration of potassium ion is about 65 mM to about 90 mM, wherein the medium is hypotonic or isotonic. In some aspects, the concentration of potassium ion is about 65 mM to about 80 mM, wherein the medium is hypotonic or isotonic. In some aspects, the concentration of potassium ion is about 65 mM to about 70 mM, wherein the medium is hypotonic or isotonic.

In some aspects, the concentration of potassium ion is higher than about 4 mM, wherein the medium is hypotonic or isotonic. In some aspects, the concentration of potassium ion is about 4 mM, wherein the medium is hypotonic or isotonic. In some aspects, the concentration of potassium ion is higher than about 5 mM, wherein the medium is hypotonic or isotonic. In some aspects, the concentration of potassium ion is about 5 mM, wherein the medium is hypotonic or isotonic. In some aspects, the concentration of potassium ion is higher than about 6 mM, wherein the medium is hypotonic or isotonic. In some aspects, the concentration of potassium ion is about 6 mM, wherein the medium is hypotonic or isotonic. In some aspects, the concentration of potassium ion is higher than about 7 mM, wherein the medium is hypotonic or isotonic. In some aspects, the concentration of potassium ion is about 7 mM, wherein the medium is hypotonic or isotonic. In some aspects, the concentration of potassium ion is higher than about 8 mM, wherein the medium is hypotonic or isotonic. In some aspects, the concentration of potassium ion is about 8 mM, wherein the medium is hypotonic or isotonic. In some aspects, the concentration of potassium ion is higher than about 9 mM, wherein the medium is hypotonic or isotonic. In some aspects, the concentration of potassium ion is about 9 mM, wherein the medium is hypotonic or isotonic.

In some aspects, the concentration of potassium ion is higher than about 10 mM, wherein the medium is hypotonic or isotonic. In some aspects, the concentration of potassium ion is about 10 mM, wherein the medium is hypotonic or isotonic. In some aspects, the concentration of potassium ion is higher than about 11 mM, wherein the medium is hypotonic or isotonic. In some aspects, the concentration of potassium ion is about 11 mM, wherein the medium is hypotonic or isotonic. In some aspects, the concentration of potassium ion is higher than about 12 mM, wherein the medium is hypotonic or isotonic. In some aspects, the concentration of potassium ion is about 12 mM, wherein the medium is hypotonic or isotonic. In some aspects, the concentration of potassium ion is higher than about 13 mM, wherein the medium is hypotonic or isotonic. In some aspects, the concentration of potassium ion is about 13 mM, wherein the medium is hypotonic. In some aspects, the concentration of potassium ion is higher than about 14 mM, wherein the medium is hypotonic or isotonic. In some aspects, the concentration of potassium ion is about 14 mM, wherein the medium is hypotonic or isotonic. In some aspects, the concentration of potassium ion is higher than about 15 mM, wherein the medium is hypotonic or isotonic. In some aspects, the concentration of potassium ion is about 15 mM, wherein the medium is hypotonic or isotonic. In some aspects, the concentration of potassium ion is higher than about 16 mM, wherein the medium is hypotonic or isotonic. In some aspects, the concentration of potassium ion is about 16 mM, wherein the medium is hypotonic or isotonic. In some aspects, the concentration of potassium ion is higher than about 17 mM, wherein the medium is hypotonic or isotonic. In some aspects, the concentration of potassium ion is about 17 mM, wherein the medium is hypotonic or isotonic. In some aspects, the concentration of potassium ion is higher than about 18 mM, wherein the medium is hypotonic or isotonic. In some aspects, the concentration of potassium ion is about 18 mM, wherein the medium is hypotonic or isotonic. In some aspects, the concentration of potassium ion is higher than about 19 mM, wherein the medium is hypotonic or isotonic. In some aspects, the concentration of potassium ion is about 19 mM, wherein the medium is hypotonic or isotonic.

In some aspects, the concentration of potassium ion is higher than about 20 mM, wherein the medium is hypotonic or isotonic. In some aspects, the concentration of potassium ion is about 20 mM, wherein the medium is hypotonic or isotonic. In some aspects, the concentration of potassium ion is higher than about 21 mM, wherein the medium is hypotonic or isotonic. In some aspects, the concentration of potassium ion is about 21 mM, wherein the medium is hypotonic or isotonic. In some aspects, the concentration of potassium ion is higher than about 22 mM, wherein the medium is hypotonic or isotonic. In some aspects, the concentration of potassium ion is about 22 mM, wherein the medium is hypotonic or isotonic. In some aspects, the concentration of potassium ion is higher than about 23 mM, wherein the medium is hypotonic or isotonic. In some aspects, the concentration of potassium ion is about 23 mM, wherein the medium is hypotonic. In some aspects, the concentration of potassium ion is higher than about 24 mM, wherein the medium is hypotonic or isotonic. In some aspects, the concentration of potassium ion is about 24 mM, wherein the medium is hypotonic or isotonic. In some aspects, the concentration of potassium ion is higher than about 25 mM, wherein the medium is hypotonic or isotonic. In some aspects, the concentration of potassium ion is about 25 mM, wherein the medium is hypotonic or isotonic. In some aspects, the concentration of potassium ion is higher than about 26 mM, wherein the medium is hypotonic or isotonic. In some aspects, the concentration of potassium ion is about 26 mM, wherein the medium is hypotonic or isotonic. In some aspects, the concentration of potassium ion is higher than about 27 mM, wherein the medium is hypotonic or isotonic. In some aspects, the concentration of potassium ion is about 27 mM, wherein the medium is hypotonic or isotonic. In some aspects, the concentration of potassium ion is higher than about 28 mM, wherein the medium is hypotonic or isotonic. In some aspects, the concentration of potassium ion is about 28 mM, wherein the medium is hypotonic or isotonic. In some aspects, the concentration of potassium ion is higher than about 29 mM, wherein the medium is hypotonic or isotonic. In some aspects, the concentration of potassium ion is about 29 mM, wherein the medium is hypotonic or isotonic.

In some aspects, the concentration of potassium ion is higher than about 30 mM, wherein the medium is hypotonic or isotonic. In some aspects, the concentration of potassium ion is about 30 mM, wherein the medium is hypotonic or isotonic. In some aspects, the concentration of potassium ion is higher than about 31 mM, wherein the medium is hypotonic or isotonic. In some aspects, the concentration of potassium ion is about 31 mM, wherein the medium is hypotonic or isotonic. In some aspects, the concentration of potassium ion is higher than about 32 mM, wherein the medium is hypotonic or isotonic. In some aspects, the concentration of potassium ion is about 32 mM, wherein the medium is hypotonic or isotonic. In some aspects, the concentration of potassium ion is higher than about 33 mM, wherein the medium is hypotonic or isotonic. In some aspects, the concentration of potassium ion is about 33 mM, wherein the medium is hypotonic. In some aspects, the concentration of potassium ion is higher than about 34 mM, wherein the medium is hypotonic or isotonic. In some aspects, the concentration of potassium ion is about 34 mM, wherein the medium is hypotonic or isotonic. In some aspects, the concentration of potassium ion is higher than about 35 mM, wherein the medium is hypotonic or isotonic. In some aspects, the concentration of potassium ion is about 35 mM, wherein the medium is hypotonic or isotonic. In some aspects, the concentration of potassium ion is higher than about 36 mM, wherein the medium is hypotonic or isotonic. In some aspects, the concentration of potassium ion is about 36 mM, wherein the medium is hypotonic or isotonic. In some aspects, the concentration of potassium ion is higher than about 37 mM, wherein the medium is hypotonic or isotonic. In some aspects, the concentration of potassium ion is about 37 mM, wherein the medium is hypotonic or isotonic. In some aspects, the concentration of potassium ion is higher than about 38 mM, wherein the medium is hypotonic or isotonic. In some aspects, the concentration of potassium ion is about 38 mM, wherein the medium is hypotonic or isotonic. In some aspects, the concentration of potassium ion is higher than about 39 mM, wherein the medium is hypotonic or isotonic. In some aspects, the concentration of potassium ion is about 39 mM, wherein the medium is hypotonic or isotonic.

In some aspects, the concentration of potassium ion is higher than about 40 mM, wherein the medium is hypotonic or isotonic. In some aspects, the concentration of potassium ion is about 40 mM, wherein the medium is hypotonic or isotonic. In some aspects, the concentration of potassium ion is higher than about 41 mM, wherein the medium is hypotonic or isotonic. In some aspects, the concentration of potassium ion is about 41 mM, wherein the medium is hypotonic or isotonic. In some aspects, the concentration of potassium ion is higher than about 42 mM, wherein the medium is hypotonic or isotonic. In some aspects, the concentration of potassium ion is about 42 mM, wherein the medium is hypotonic or isotonic. In some aspects, the concentration of potassium ion is higher than about 43 mM, wherein the medium is hypotonic or isotonic. In some aspects, the concentration of potassium ion is about 43 mM, wherein the medium is hypotonic. In some aspects, the concentration of potassium ion is higher than about 44 mM, wherein the medium is hypotonic or isotonic. In some aspects, the concentration of potassium ion is about 44 mM, wherein the medium is hypotonic or isotonic. In some aspects, the concentration of potassium ion is higher than about 45 mM, wherein the medium is hypotonic or isotonic. In some aspects, the concentration of potassium ion is about 45 mM, wherein the medium is hypotonic or isotonic. In some aspects, the concentration of potassium ion is higher than about 46 mM, wherein the medium is hypotonic or isotonic. In some aspects, the concentration of potassium ion is about 46 mM, wherein the medium is hypotonic or isotonic. In some aspects, the concentration of potassium ion is higher than about 47 mM, wherein the medium is hypotonic or isotonic. In some aspects, the concentration of potassium ion is about 47 mM, wherein the medium is hypotonic or isotonic. In some aspects, the concentration of potassium ion is higher than about 48 mM, wherein the medium is hypotonic or isotonic. In some aspects, the concentration of potassium ion is about 48 mM, wherein the medium is hypotonic or isotonic. In some aspects, the concentration of potassium ion is higher than about 49 mM, wherein the medium is hypotonic or isotonic. In some aspects, the concentration of potassium ion is about 49 mM, wherein the medium is hypotonic or isotonic.

In some aspects, the metabolic reprogramming medium comprising a high concentration of potassium ion is prepared by adding a sufficient amount of a potassium salt in a medium. In some aspects, non-limiting examples of potassium salt include potassium aminetrichloroplatinate, potassium aquapentachlororuthenate, potassium bis(oxalato)platinate(II) dihydrate, potassium bisulfate, potassium borohydride, potassium bromide, potassium carbonate, potassium chloride, potassium chromate, potassium dichromate, potassium dicyanoargentate, potassium dicyanoaurate, potassium fluoride, potassium fluorosulfate, potassium hexachloroiridate, potassium hexachloroosmate, potassium hexachloropalladate, potassium hexachloroplatinate, potassium hexachlororhenate, potassium hexacyanochromate, potassium hexacyanoferrate, potassium hexacyanoruthenate(II) hydrate, potassium hexafluoroantimonate, potassium hexafluoronickelate, potassium hexafluorophosphate, potassium hexafluorotitanate, potassium hexafluorozirconate, potassium hexahydroxoantimonate, potassium hexaiodoplatinate, potassium hexaiodorhenate, potassium hydroxide, potassium iodate, potassium iodide, potassium manganate, potassium metavanadate, potassium molybdate, potassium nitrate, potassium nitrosodisulfonate, potassium osmate(VI) dihydrate, potassium pentachloronitrosylruthenate, potassium perchlorate, potassium perrhenate, potassium perruthenate, potassium persulfate, potassium phosphate dibasic, potassium phosphate monobasic, potassium pyrophosphate, potassium selenocyanate, potassium selenocyanate, potassium stannate trihydrate, potassium sulfate, potassium tellurate hydrate, potassium tellurite, potassium tetraborate tetrahydrate, potassium tetrabromoaurate, potassium tetrabromopalladate, potassium tetrachloropalladate, potassium tetrachloroplatinate, potassium tetracyanopalladate, potassium tetracyanoplatinate, potassium tetrafluoroborate, potassium tetranitroplatinate, potassium tetrathionate, potassium p-toluenethiosulfonate, potassium hydroxycitrate tribasic monohydrate, or any combination thereof. In certain aspects, the potassium salt comprises potassium chloride (KCl). In certain aspects, the potassium salt comprises potassium gluconate. In certain aspects, the potassium salt comprises potassium citrate. In certain aspects, the potassium salt comprises potassium hydroxycitrate.

II.A.2. Sodium

Some aspects of the present disclosure are directed to methods of culturing immune cells, e.g., T cells and/or NK cells, comprising contacting the immune cells with PCS in a medium comprising (i) potassium ion at a concentration of at least about 5 mM and (ii) sodium ion (e.g., NaCl) at a concentration of less than about 115 mM. In some aspects, the medium is hypotonic or isotonic. In some aspects, the target concentration of sodium (e.g., NaCl) is reached by starting with a basal medium comprising a higher concentration of sodium ion (e.g., NaCl), and diluting the solution to reach the target concentration of sodium ion (e.g., NaCl). In some aspects, the target concentration of sodium ion (e.g., NaCl) is reached by adding one or more sodium salts (e.g., more NaCl). Non-limiting examples of sodium salts include sodium (meta)periodate, sodium arsenyl tartrate hydrate, sodium azide, sodium benzyloxide, sodium bromide, sodium carbonate, sodium chloride, sodium chromate, sodium cyclohexanebutyrate, sodium ethanethiolate, sodium fluoride, sodium fluorophosphate, sodium formate, sodium hexachloroiridate(III) hydrate, sodium hexachloroiridate(IV) hexahydrate, sodium hexachloroplatinate(IV) hexahydrate, sodium hexachlororhodate(III), sodium hexafluoroaluminate, sodium hexafluoroantimonate(V), sodium hexafluoroarsenate(V), sodium hexafluoroferrate(III), sodium hexafluorophosphate, sodium hexafluorosilicate, sodium hexahydroxyplatinate(IV), sodium hexametaphosphate, sodium hydrogen difluoride, sodium hydrogen sulfate, sodium hydrogencyanamide, sodium hydroxide, sodium iodide, sodium metaborate tetrahydrate, sodium metasilicate nonahydrate, sodium metavanadate, sodium molybdate, sodium nitrate, sodium nitrite, sodium oxalate, sodium perborate monohydrate, sodium percarbonate, sodium perchlorate, sodium periodate, sodium permanganate, sodium perrhenate, sodium phosphate, sodium pyrophosphate, sodium selenate, sodium selenite, sodium stannate, sodium sulfate, sodium tellurite, sodium tetraborate, sodium tetrachloroaluminate, sodium tetrachloroaurate(III), sodium tetrachloropalladate(II), sodium tetrachloroplatinate(II), sodium thiophosphate tribasic, sodium thiosulfate, sodium thiosulfate pentahydrate, sodium yttrium oxyfluoride, Trisodium trimetaphosphate, or any combination thereof. In some aspects, the sodium salt comprises sodium chloride (NaCl). In some aspects, the sodium salt comprises sodium gluconate. In some aspects, the sodium salt comprises sodium bicarbonate. In some aspects, the sodium salt comprises sodium hydroxycitrate. In some aspects, the sodium salt comprises sodium phosphate.

In some aspects, the concentration of the sodium ion (e.g., NaCl) in a metabolic reprogramming medium of the present disclosure is less than that of the basal medium. In some aspects, the concentration of the sodium ion (e.g., NaCl) is reduced as the concentration of potassium ion is increased. In some aspects, the concentration of the sodium ion (e.g., NaCl) is from about 25 mM to about 115 mM. In some aspects, the concentration of the sodium (e.g., NaCl) ion is from about 25 mM to about 100 mM, about 30 mM to about 40 mM, about 30 mM to about 50 mM, about 30 mM to about 60 mM, about 30 mM to about 70 mM, about 30 mM to about 80 mM, about 40 mM to about 50 mM, about 40 mM to about 60 mM, about 40 mM to about 70 mM, about 40 mM to about 80 mM, about 50 mM to about 55 mM, about 50 mM to about 60 mM, about 50 mM to about 65 mM, about 50 mM to about 70 mM, about 50 mM to about 75 mM, about 50 mM to about 80 mM, about 55 mM to about 60 mM, about 55 mM to about 65 mM, about 55 mM to about 70 mM, about 55 mM to about 75 mM, about 55 mM to about 80 mM, about 60 mM to about 65 mM, about 60 mM to about 70 mM, about 60 mM to about 75 mM, about 60 mM to about 80 mM, about 70 mM to about 75 mM, about 70 mM to about 80 mM, or about 75 mM to about 80 mM. In some aspects, the concentration of the sodium ion (e.g., NaCl) is from about 40 mM to about 80 mM. In some aspects, the concentration of the sodium ion (e.g., NaCl) is from about 50 mM to about 85 mM. In some aspects, the concentration of the sodium ion (e.g., NaCl) is from about 55 mM to about 80 mM. In some aspects, the concentration of the sodium ion (e.g., NaCl) is from about 30 mM to about 35 mM. In some aspects, the concentration of the sodium ion (e.g., NaCl) is from about 35 mM to about 40 mM. In some aspects, the concentration of the sodium ion (e.g., NaCl) is from about 40 mM to about 45 mM. In some aspects, the concentration of the sodium ion (e.g., NaCl) is from about 45 mM to about 50 mM. In some aspects, the concentration of the sodium ion (e.g., NaCl) is from about 50 mM to about 55 mM. In some aspects, the concentration of the sodium ion (e.g., NaCl) is from about 55 mM to about 60 mM. In some aspects, the concentration of the sodium ion (e.g., NaCl) is from about 60 mM to about 65 mM. In some aspects, the concentration of the sodium ion (e.g., NaCl) is from about 65 mM to about 70 mM. In some aspects, the concentration of the sodium ion (e.g., NaCl) is from about 70 mM to about 75 mM. In some aspects, the concentration of the sodium ion (e.g., NaCl) is from about 75 mM to about 80 mM. In some aspects, the concentration of the sodium ion (e.g., NaCl) is from about 80 mM to about 85 mM.

In some aspects, the concentration of the sodium ion (e.g., NaCl) is about 30 mM, about 35 mM, about 40 mM, about 45 mM, about 50 mM, about 55 mM, about 60 mM, about 65 mM, about 70 mM, about 75 mM, about 80 mM, about 85 mM, or about 90 mM. In certain aspects, the concentration of sodium ion (e.g., NaCl) is about 40 mM. In some aspects, the concentration of sodium ion (e.g., NaCl) is about 45 mM. In some aspects, the concentration of sodium ion (e.g., NaCl) is about 50 mM. In some aspects, the concentration of sodium ion (e.g., NaCl) is about 55 mM. In some aspects, the concentration of sodium ion (e.g., NaCl) is about 55.6 mM. In some aspects, the concentration of sodium ion (e.g., NaCl) is about 59.3 mM. In some aspects, the concentration of sodium ion (e.g., NaCl) is about 60 mM. In some aspects, the concentration of sodium ion (e.g., NaCl) is about 63.9 mM. In some aspects, the concentration of sodium ion (e.g., NaCl) is about 65 mM. In some aspects, the concentration of sodium ion (e.g., NaCl) is about 67.6 mM. In some aspects, the concentration of sodium ion (e.g., NaCl) is about 70 mM. In some aspects, the concentration of sodium ion (e.g., NaCl) is about 72.2 mM. In some aspects, the concentration of sodium ion (e.g., NaCl) is about 75 mM. In some aspects, the concentration of sodium ion (e.g., NaCl) is about 76 mM. In some aspects, the concentration of sodium ion (e.g., NaCl) is about 80 mM. In some aspects, the concentration of sodium ion (e.g., NaCl) is about 80.5 mM. In some aspects, the metabolic reprogramming medium comprises about 40 mM to about 90 mM potassium ion and about 40 mM to about 80 mM sodium ion (e.g., NaCl).

In some aspects, the metabolic reprogramming medium comprises about 50 mM to about 75 mM potassium ion and about 80 mM to about 90 mM sodium ion (e.g., NaCl). In some aspects, the metabolic reprogramming medium comprises about 55 mM to about 75 mM potassium ion and about 80 mM to about 90 mM sodium ion (e.g., NaCl). In some aspects, the metabolic reprogramming medium comprises about 60 mM to about 75 mM potassium ion and about 80 mM to about 90 mM sodium ion (e.g., NaCl). In some aspects, the metabolic reprogramming medium comprises about 65 mM to about 75 mM potassium ion and about 80 mM to about 85 mM sodium ion (e.g., NaCl). In some aspects, the metabolic reprogramming medium comprises about 65 mM potassium ion and about 80 mM to about 85 mM sodium ion (e.g., NaCl). In some aspects, the metabolic reprogramming medium comprises about 66 mM potassium ion and about 80 mM to about 85 mM sodium ion (e.g., NaCl). In some aspects, the metabolic reprogramming medium comprises about 67 mM potassium ion and about 80 mM to about 85 mM sodium ion (e.g., NaCl). In some aspects, the metabolic reprogramming medium comprises about 68 mM potassium ion and about 80 mM to about 85 mM sodium ion (e.g., NaCl). In some aspects, the metabolic reprogramming medium comprises about 69 mM potassium ion and about 80 mM to about 85 mM sodium ion (e.g., NaCl). In some aspects, the metabolic reprogramming medium comprises about 70 mM potassium ion and about 80 mM to about 85 mM sodium ion (e.g., NaCl). In some aspects, the metabolic reprogramming medium comprises about 71 mM potassium ion and about 80 mM to about 85 mM sodium ion (e.g., NaCl). In some aspects, the metabolic reprogramming medium comprises about 72 mM potassium ion and about 80 mM to about 85 mM sodium ion (e.g., NaCl). In some aspects, the metabolic reprogramming medium comprises about 73 mM potassium ion and about 80 mM to about 85 mM sodium ion (e.g., NaCl). In some aspects, the metabolic reprogramming medium comprises about 74 mM potassium ion and about 80 mM to about 85 mM sodium ion (e.g., NaCl). In some aspects, the metabolic reprogramming medium comprises about 75 mM potassium ion and about 80 mM to about 85 mM sodium ion (e.g., NaCl). In some aspects, the metabolic reprogramming medium comprises about 65 mM potassium ion and about 80 mM sodium ion (e.g., NaCl). In some aspects, the metabolic reprogramming medium comprises about 65 mM potassium ion and about 85 mM sodium ion (e.g., NaCl). In some aspects, the metabolic reprogramming medium comprises about 65 mM potassium ion and about 90 mM sodium ion (e.g., NaCl). In some aspects, the metabolic reprogramming medium comprises about 70 mM potassium ion and about 80 mM sodium ion (e.g., NaCl). In some aspects, the metabolic reprogramming medium comprises about 70 mM potassium ion and about 85 mM sodium ion (e.g., NaCl). In some aspects, the metabolic reprogramming medium comprises about 70 mM potassium ion and about 90 mM sodium ion (e.g., NaCl). In some aspects, the metabolic reprogramming medium comprises about 75 mM potassium ion and about 80 mM sodium ion (e.g., NaCl). In some aspects, the metabolic reprogramming medium comprises about 75 mM potassium ion and about 85 mM sodium ion (e.g., NaCl). In some aspects, the metabolic reprogramming medium comprises about 75 mM potassium ion and about 90 mM sodium ion (e.g., NaCl).

In some aspects, the metabolic reprogramming medium comprises about 40 mM to about 90 mM potassium ion and about 30 mM to about 109 mM NaCl, wherein the concentration of NaCl (mM) is equal to or lower than (135−potassium ion concentration, meaning 135 minus the concentration of potassium ion). In some aspects, the metabolic reprogramming medium comprises about 40 mM potassium ion and less than or equal to about 95 mM NaCl (e.g., about 95 mM, about 94 mM, about 93 mM, about 92 mM, about 91 mM, about 90 mM, about 85 mM, about 80 mM, about 75 mM, about 70 mM, about 65 mM, about 60 mM, about 55 mM, or about 50 mM NaCl). In some aspects, the metabolic reprogramming medium comprises about 45 mM potassium ion and less than or equal to about 90 mM NaCl (e.g., about 90 mM, about 89 mM, about 88 mM, about 87 mM, about 86 mM, about 85 mM, about 80 mM, about 75 mM, about 70 mM, about 65 mM, about 60 mM, about 55 mM, or about 50 mM NaCl). In some aspects, the metabolic reprogramming medium comprises about 50 mM potassium ion and less than or equal to about 85 mM NaCl (e.g., about 85 mM, about 84 mM, about 83 mM, about 82 mM, about 81 mM, about 80 mM, about 75 mM, about 70 mM, about 65 mM, about 60 mM, about 55 mM, or about 50 mM NaCl). In some aspects, the metabolic reprogramming medium comprises about 55 mM potassium ion and less than or equal to about 80 mM NaCl (e.g., about 80 mM, about 79 mM, about 78 mM, about 77 mM, about 76 mM, about 75 mM, about 70 mM, about 65 mM, about 60 mM, about 55 mM, or about 50 mM NaCl). In some aspects, the metabolic reprogramming medium comprises about 60 mM potassium ion and less than or equal to about 75 mM NaCl (e.g., about 75 mM, about 74 mM, about 73 mM, about 72 mM, about 71 mM, about 70 mM, about 65 mM, about 60 mM, about 55 mM, or about 50 mM NaCl). In some aspects, the metabolic reprogramming medium comprises about 65 mM potassium ion and less than or equal to about 70 mM NaCl (e.g., about 70 mM, about 69 mM, about 68 mM, about 67 mM, about 66 mM, about 65 mM, about 60 mM, about 55 mM, or about 50 mM NaCl). In some aspects, the metabolic reprogramming medium comprises about 70 mM potassium ion and less than or equal to about 70 mM NaCl (e.g., about 65 mM, about 64 mM, about 63 mM, about 62 mM, about 61 mM, about 60 mM, about 55 mM, or about 50 mM NaCl). In some aspects, the metabolic reprogramming medium comprises about 75 mM potassium ion and less than or equal to about 60 mM NaCl (e.g., about 60 mM, about 59 mM, about 58 mM, about 57 mM, about 56 mM, about 55 mM, about 50 mM, about 45 mM, or about 40 mM NaCl). In some aspects, the metabolic reprogramming medium comprises about 80 mM potassium ion and less than or equal to about 55 mM NaCl (e.g., about 55 mM, about 54 mM, about 53 mM, about 52 mM, about 51 mM, about 50 mM, about 45 mM, about 40 mM, or about 35 mM NaCl). In some aspects, the metabolic reprogramming medium comprises about 85 mM potassium ion and less than or equal to about 50 mM NaCl (e.g., about 50 mM, about 49 mM, about 48 mM, about 47 mM, about 46 mM, about 45 mM, about 40 mM, about 35 mM, or about 30 mM NaCl). In some aspects, the metabolic reprogramming medium comprises about 90 mM potassium ion and less than or equal to about 45 mM NaCl (e.g., about 45 mM, about 44 mM, about 43 mM, about 42 mM, about 41 mM, about 40 mM, about 35 mM, about 30 mM, or about 25 mM NaCl). In some aspects, the metabolic reprogramming medium comprises about 70 mM potassium ion and about 60 mM NaCl. In some aspects, the metabolic reprogramming medium comprises about 70 mM potassium ion and about 61 mM NaCl. In some aspects, the metabolic reprogramming medium comprises about 70 mM potassium ion and about 62 mM NaCl.

In some aspects, the medium comprises about 50 mM potassium ion and about 75 mM NaCl. In some aspects, the medium is hypotonic. In some aspects, the medium is isotonic.

Some aspects of the present disclosure are directed to methods of culturing immune cells (e.g., T cells and/or NK cells) comprising contacting the immune cells with PCS in a medium comprising (i) potassium ion at a concentration higher than 5 mM and (ii) NaCl at a concentration of less than about 135 mM. Some aspects of the present disclosure are directed to methods of culturing immune cells, e.g., T cells and/or NK cells, comprising contacting the immune cells with PCS in a medium comprising (i) potassium ion at a concentration higher than 40 mM and (ii) NaCl at a concentration of less than about 100 mM. Some aspects of the present disclosure are directed to methods of culturing immune cells, e.g., T cells and/or NK cells, comprising contacting the immune cells with PCS in a medium comprising (i) potassium ion at a concentration higher than 50 mM and (ii) NaCl at a concentration of less than about 90 mM. Some aspects of the present disclosure are directed to methods of culturing immune cells, e.g., T cells and/or NK cells, comprising contacting the immune cells with PCS in a medium comprising (i) potassium ion at a concentration higher than 55 mM and (ii) NaCl at a concentration of less than about 70 mM. Some aspects of the present disclosure are directed to methods of culturing immune cells, e.g., T cells and/or NK cells, comprising contacting the immune cells with PCS in a medium comprising (i) potassium ion at a concentration higher than 60 mM and (ii) NaCl at a concentration of less than about 70 mM.

II.A.3. Tonicity

In some aspects of the present disclosure, the tonicity of the metabolic reprogramming medium (e.g., (concentration of potassium ion and concentration of NaCl)×2) is adjusted based on the concentration of potassium ion and/or NaCl. In some aspects, the tonicity of the metabolic reprogramming medium is lower than that of the basal medium. In some aspects, the tonicity of the metabolic reprogramming medium is higher than that of the basal medium. In some aspect, the tonicity of the medium is the same as that of the basal medium. The tonicity of the metabolic reprogramming medium can be affected by modifying the concentration of potassium ion and/or NaCl in the media. In some aspects, increased potassium ion concentration is paired with an increase or a decrease in the concentration of NaCl. In some aspects, this pairing affects the tonicity of the metabolic reprogramming medium. In some aspects, the concentration of potassium ion is increased while the concentration of NaCl, is decreased.

In some aspects, the medium useful for the present media is prepared based on the function of potassium ion and tonicity. For example, in some aspects, if the medium useful for the present disclosure is hypotonic (e.g., less than 280 mOsm) and comprises at least about 50 mM of potassium ion, a concentration of NaCl that is sufficient to maintain the medium as hypotonic can be determined based on the following formula: NaCl concentration=(desired tonicity (280)/2)−potassium ion concentration. (i.e., the concentration of NaCl (mM) is equal to or lower than (140−potassium ion concentration)). In some aspects, a hypotonic medium disclosed herein comprises a total concentration of potassium ion and NaCl between 110 mM and 140 mM. Therefore, for hypotonic medium, the concentration of potassium ion can be set at a concentration between 50 mM and 90 mM, and the NaCl concentration can be between 90 mM and 50 mM, or lower, so long as the total concentration of potassium ion and NaCl is between 110 mM and 140 mM. In some aspects, a hypotonic medium disclosed herein comprises a total concentration of potassium ion and NaCl between 115 mM and 140 mM. In some aspects, the hypotonic medium disclosed herein comprises a total concentration of potassium ion and NaCl between 120 mM and 140 mM.

In some aspects, the metabolic reprogramming medium is isotonic (between 280 mOsm and 300 mOsm) and comprises a concentration of potassium ion between about 50 mM and 70 mM. The corresponding concentration of NaCl can be again calculated based on the formula: NaCl concentration=(desired tonicity/2)−potassium ion concentration. For example, if the concentration of potassium is 50 mM and the desired tonicity is 300 mOsm, the NaCl concentration can be 100 mM.

In some aspects, the metabolic reprogramming medium is isotonic. In some aspects, the metabolic reprogramming medium has a tonicity of about 280 mOsm/L. In some aspects, the metabolic reprogramming medium has a tonicity of 280 mOsm/L. In some aspects, the metabolic reprogramming medium has a tonicity of 280 mOsm/L±1 mOsm/L. In some aspects, the metabolic reprogramming medium has a tonicity of 280 mOsm/L±2 mOsm/L. In some aspects, the metabolic reprogramming medium has a tonicity of 280 mOsm/L±3 mOsm/L. In some aspects, the metabolic reprogramming medium has a tonicity of 280 mOsm/L±4 mOsm/L. In some aspects, the metabolic reprogramming medium has a tonicity of 280 mOsm/L±5 mOsm/L. In some aspects, the metabolic reprogramming medium has a tonicity of 280 mOsm/L±6 mOsm/L. In some aspects, the metabolic reprogramming medium has a tonicity of 280 mOsm/L±7 mOsm/L. In some aspects, the metabolic reprogramming medium has a tonicity of 280 mOsm/L±8 mOsm/L. In some aspects, the metabolic reprogramming medium has a tonicity of 280 mOsm/L±19 mOsm/L. In some aspects, the metabolic reprogramming medium has a tonicity of 280 mOsm/L±10 mOsm/L. In some aspects, the metabolic reprogramming medium has a tonicity of about 280 mOsm/L to about 285 mOsm/L, about 280 mOsm/L to about 290 mOsm/L, about 280 mOsm/L to about 295 mOsm/L, about 280 mOsm/L to about 300 mOsm/L, about 280 mOsm/L to about 305 mOsm/L, about 280 mOsm/L to about 310 mOsm/L, about 280 mOsm/L to about 315 mOsm/L, or about 280 mOsm/L to less than 320 mOsm/L. In some aspects, the metabolic reprogramming medium has a tonicity of about 285 mOsm/L, about 290 mOsm/L, about 295 mOsm/L, about 300 mOsm/L, about 305 mOsm/L, about 310 mOsm/L, or about 315 mOsm/L.

In some aspects, the metabolic reprogramming medium is hypotonic. In some aspects, the metabolic reprogramming medium has a tonicity lower than about 280 mOsm/L. In some aspects, the metabolic reprogramming medium has a tonicity lower than about 280 mOsm/L; as measured by adding the potassium ion concentration and the NaCl concentration, and multiplying by two. In some aspects, the metabolic reprogramming medium has a tonicity lower than 280 mOsm/L. In some aspects, the metabolic reprogramming medium has a tonicity lower than 280 mOsm/L; as measured by adding the potassium ion concentration and the NaCl concentration, and multiplying by two. In some aspects, the metabolic reprogramming medium has a tonicity lower than 275 mOsm/L. In some aspects, the metabolic reprogramming medium has a tonicity lower than 275 mOsm/L; as measured by adding the potassium ion concentration and the NaCl concentration, and multiplying by two; as measured by adding the potassium ion concentration and the NaCl concentration, and multiplying by two. In some aspects, the metabolic reprogramming medium has a tonicity lower than 270 mOsm/L. In some aspects, the metabolic reprogramming medium has a tonicity lower than 270 mOsm/L; as measured by adding the potassium ion concentration and the NaCl concentration, and multiplying by two. In some aspects, the metabolic reprogramming medium has a tonicity lower than 265 mOsm/L. In some aspects, the metabolic reprogramming medium has a tonicity lower than 265 mOsm/L; as measured by adding the potassium ion concentration and the NaCl concentration, and multiplying by two. In some aspects, the metabolic reprogramming medium has a tonicity lower than 260 mOsm/L. In some aspects, the metabolic reprogramming medium has a tonicity lower than 260 mOsm/L; as measured by adding the potassium ion concentration and the NaCl concentration, and multiplying by two. In some aspects, the metabolic reprogramming medium has a tonicity lower than 265 mOsm/L. In some aspects, the metabolic reprogramming medium has a tonicity lower than 265 mOsm/L; as measured by adding the potassium ion concentration and the NaCl concentration, and multiplying by two. In some aspects, the metabolic reprogramming medium has a tonicity lower than 260 mOsm/L. In some aspects, the metabolic reprogramming medium has a tonicity lower than 260 mOsm/L; as measured by adding the potassium ion concentration and the NaCl concentration, and multiplying by two. In some aspects, the metabolic reprogramming medium has a tonicity lower than 255 mOsm/L. In some aspects, the metabolic reprogramming medium has a tonicity lower than 255 mOsm/L; as measured by adding the potassium ion concentration and the NaCl concentration, and multiplying by two. In some aspects, the metabolic reprogramming medium has a tonicity lower than about 250 mOsm/L. In some aspects, the metabolic reprogramming medium has a tonicity lower than about 250 mOsm/L; as measured by adding the potassium ion concentration and the NaCl concentration, and multiplying by two. In some aspects, the metabolic reprogramming medium has a tonicity lower than about 245 mOsm/L. In some aspects, the metabolic reprogramming medium has a tonicity lower than about 245 mOsm/L; as measured by adding the potassium ion concentration and the NaCl concentration, and multiplying by two. In some aspects, the metabolic reprogramming medium has a tonicity lower than about 240 mOsm/L. In some aspects, the metabolic reprogramming medium has a tonicity lower than about 240 mOsm/L; as measured by adding the potassium ion concentration and the NaCl concentration, and multiplying by two. In some aspects, the metabolic reprogramming medium has a tonicity lower than about 235 mOsm/L. In some aspects, the metabolic reprogramming medium has a tonicity lower than about 235 mOsm/L; as measured by adding the potassium ion concentration and the NaCl concentration, and multiplying by two. In some aspects, the metabolic reprogramming medium has a tonicity lower than about 230 mOsm/L. In some aspects, the metabolic reprogramming medium has a tonicity lower than about 230 mOsm/L; as measured by adding the potassium ion concentration and the NaCl concentration, and multiplying by two. In some aspects, the metabolic reprogramming medium has a tonicity lower than about 225 mOsm/L. In some aspects, the metabolic reprogramming medium has a tonicity lower than about 225 mOsm/L. In some aspects, the tonicity is higher than about 220 mOsm/L; as measured by adding the potassium ion concentration and the NaCl concentration, and multiplying by two. In some aspects, the metabolic reprogramming medium has a tonicity from about 230 mOsm/L to about 280 mOsm/L. In some aspects, the metabolic reprogramming medium has a tonicity from about 240 mOsm/L to about 280 mOsm/L.

In some aspects, the metabolic reprogramming medium has an osmolality lower than about 220 mOsm/L. In some aspects, the metabolic reprogramming medium has an osmolality lower than about 215 mOsm/L. In some aspects, the metabolic reprogramming medium has an osmolality lower than about 210 mOsm/L. In some aspects, the metabolic reprogramming medium has an osmolality lower than about 205 mOsm/L. In some aspects, the metabolic reprogramming medium has an osmolality lower than about 200 mOsm/L.

In some aspects, the metabolic reprogramming medium has a tonicity from about 100 mOsm/L to about 280 mOsm/L, about 125 mOsm/L to about 280 mOsm/L, about 150 mOsm/L to about 280 mOsm/L, about 175 mOsm/L to about 280 mOsm/L, about 200 mOsm/L to about 280 mOsm/L, about 210 mOsm/L to about 280 mOsm/L, about 220 mOsm/L to about 280 mOsm/L, about 225 mOsm/L to about 280 mOsm/L, about 230 mOsm/L to about 280 mOsm/L, about 235 mOsm/L to about 280 mOsm/L, about 240 mOsm/L to about 280 mOsm/L, about 245 mOsm/L to about 280 mOsm/L, about 250 mOsm/L to about 280 mOsm/L, about 255 mOsm/L to about 280 mOsm/L, about 260 mOsm/L to about 280 mOsm/L, about 265 mOsm/L to about 280 mOsm/L, about 270 mOsm/L to about 280 mOsm/L, or about 275 mOsm/L to about 280 mOsm/L. In some aspects, the metabolic reprogramming medium has a tonicity from about 250 mOsm/L to about 270 mOsm/L. In some aspects, the metabolic reprogramming medium has a tonicity from about 250 mOsm/L to about 255 mOsm/L, about 250 mOsm/L to about 260 mOsm/L, about 250 mOsm/L to about 265 mOsm/L, about 255 mOsm/L to about 260 mOsm/L, about 255 mOsm/L to about 265 mOsm/L, about 255 mOsm/L to about 265 mOsm/L, about 260 mOsm/L to about 265 mOsm/L, or about 254 mOsm/L to about 263 mOsm/L. In some aspects, the metabolic reprogramming medium has a tonicity from about 254 mOsm/L to about 255 mOsm/L. In some aspects, the metabolic reprogramming medium has a tonicity from about 255 mOsm/L to about 256 mOsm/L. In some aspects, the metabolic reprogramming medium has a tonicity from about 256 mOsm/L to about 257 mOsm/L. In some aspects, the metabolic reprogramming medium has a tonicity from about 257 mOsm/L to about 258 mOsm/L. In some aspects, the metabolic reprogramming medium has a tonicity from about 258 mOsm/L to about 259 mOsm/L. In some aspects, the metabolic reprogramming medium has a tonicity from about 260 mOsm/L to about 261 mOsm/L. In some aspects, the metabolic reprogramming medium has a tonicity from about 261 mOsm/L to about 262 mOsm/L. In some aspects, the metabolic reprogramming medium has a tonicity from about 262 mOsm/L to about 263 mOsm/L. In some aspects, the metabolic reprogramming medium has a tonicity from about 263 mOsm/L to about 264 mOsm/L. In some aspects, the metabolic reprogramming medium has a tonicity from about 264 mOsm/L to about 265 mOsm/L. In some aspects, the metabolic reprogramming medium has a tonicity from about 220 mOsm/L to about 280 mOsm/L.

In some aspects, the metabolic reprogramming medium has a tonicity of about 100 mOsm/L, about 125 mOsm/L, about 150 mOsm/L, about 175 mOsm/L, about 200 mOsm/L, about 210 mOsm/L, about 220 mOsm/L, about 225 mOsm/L, about 230 mOsm/L, about 235 mOsm/L, about 240 mOsm/L, about 245 mOsm/L, about 250 mOsm/L, about 255 mOsm/L, about 260 mOsm/L, about 265 mOsm/L, about 270 mOsm/L, or about 275 mOsm/L.

In some aspects, the metabolic reprogramming medium has a tonicity of about 250 mOsm/L. In some aspects, the metabolic reprogramming medium has a tonicity of about 262.26 mOsm/L. In some aspects, the metabolic reprogramming medium has a tonicity of about 260 mOsm/L. In some aspects, the metabolic reprogramming medium has a tonicity of about 259.7 mOsm/L. In some aspects, the metabolic reprogramming medium has a tonicity of about 257.5 mOsm/L. In some aspects, the metabolic reprogramming medium has a tonicity of about 257.2 mOsm/L. In some aspects, the metabolic reprogramming medium has a tonicity of about 255.2 mOsm/L. In some aspects, the metabolic reprogramming medium has a tonicity of about 254.7. In some aspects, the metabolic reprogramming medium has a tonicity of about 255 mOsm/L. In some aspects, the metabolic reprogramming medium has a tonicity of about 260 mOsm/L.

In some aspects, the metabolic reprogramming medium comprises about 50 mM potassium ion and (i) about 80.5 mM NaCl; (ii) about 17.7 mM glucose; and (iii) about 1.8 mM calcium ion. In some aspects, the metabolic reprogramming medium comprises about 55 mM potassium ion and (i) about 76 mM NaCl; (ii) about 17.2 mM glucose; and (iii) about 1.7 mM calcium ion. In some aspects, the metabolic reprogramming medium comprises about 60 mM potassium ion and (i) about 72.2 mM NaCl; (ii) about 16.8 mM glucose; and (iii) about 1.6 mM calcium ion. In some aspects, the metabolic reprogramming medium comprises about 65 mM potassium ion and (i) about 67.6 mM NaCl; (ii) about 16.3 mM glucose; and (iii) about 1.5 mM calcium ion. In some aspects, the metabolic reprogramming medium comprises about 70 mM potassium ion and (i) about 63.9 mM NaCl; (ii) about 15.9 mM glucose; and (iii) about 1.4 mM calcium ion. In some aspects, the metabolic reprogramming medium comprises about 75 mM potassium ion and (i) about 59.3 mM NaCl; (ii) about 15.4 mM glucose; and (iii) about 1.3 mM calcium ion. In some aspects, the metabolic reprogramming medium comprises about 80 mM potassium ion and (i) about 55.6 mM NaCl; (ii) about 15 mM glucose; and (iii) about 1.2 mM calcium ion. The tonicity of the metabolic reprogramming medium can be adjusted, e.g., to an isotonic or hypotonic state disclosed herein, at any point. In some aspects, the tonicity of the metabolic reprogramming medium can be adjusted, e.g., to an isotonic or hypotonic state disclosed herein, before the cells are added to the metabolic reprogramming medium. In some aspects, the cells are cultured in the hypotonic or isotonic medium prior to cell engineering, e.g., prior to transduction with a construct expressing a CAR, TCR or TCR mimic. In some aspects, the cells are cultured in the hypotonic or isotonic medium during cell engineering, e.g., during transduction with a construct expressing a CAR, TCR or TCR mimic. In some aspects the cells are cultured in the hypotonic or isotonic medium after cell engineering, e.g., after transduction with a construct expressing a CAR, TCR or TCR mimic. In some aspects, the cells are cultured in the hypotonic or isotonic medium throughout cell expansion.

II.A.4. Saccharides

Some aspects of the present disclosure are directed to methods of culturing immune cells, e.g., T cells and/or NK cells, comprising contacting the immune cells with PCS in a medium comprising (i) potassium ion at a concentration of at least about 5 mM and (ii) a saccharide. In some aspects, the medium is hypotonic or isotonic.

In some aspects, the target concentration of the saccharide is reached by starting with a basal medium comprising a higher concentration of the saccharide, and diluting the solution to reach the target concentration of the saccharide. In some aspects, the target concentration of the saccharide is reached by raising the concentration of the saccharide by adding the saccharide until the desired concentration is reached. In some aspects, the saccharide is a monosaccharide, a disaccharide, or a polysaccharide. In some aspects, the saccharide is selected from glucose, fructose, galactose, mannose, maltose, sucrose, lactose, trehalose, or any combination thereof. In certain aspects, the saccharide is glucose. In some aspects, the medium comprises (i) potassium ion at a concentration of at least about 5 mM and (ii) glucose. In some aspects, the medium comprises (i) potassium ion at a concentration higher than 40 mM and (ii) glucose. In some aspects, the medium comprises (i) potassium ion at a concentration of at least about 5 mM and (ii) mannose. In some aspects, the medium comprises (i) potassium ion at a concentration of at least about 50 mM and (ii) mannose. In some aspects, the medium is hypotonic. In some aspects, the medium is isotonic. In some aspects, the medium comprises (i) potassium ion at a concentration higher than 40 mM and (ii) glucose; wherein the total concentration of potassium ion and NaCl is between 110 mM and 140 mM. In some aspects, the medium comprises (i) potassium ion at a concentration higher than 50 mM and (ii) glucose; wherein the total concentration of potassium ion and NaCl is between 110 mM and 140 mM. In some aspects, the medium comprises (i) potassium ion at a concentration of at least about 40 mM and (ii) mannose; wherein the total concentration of potassium ion and NaCl is between 110 mM and 140 mM. In some aspects, the medium comprises (i) potassium ion at a concentration of at least about 50 mM and (ii) mannose; wherein the total concentration of potassium ion and NaCl is between 110 mM and 140 mM.

In some aspects, the metabolic reprogramming medium comprises (i) potassium ion at a concentration higher than 5 mM and (ii) glucose. In some aspects, the metabolic reprogramming medium comprises (i) potassium ion at a concentration of at least about 30 mM to at least about 100 mM and (ii) glucose. In some aspects, the metabolic reprogramming medium comprises (i) potassium ion at a concentration higher than 40 mM and (ii) glucose. In some aspects, the metabolic reprogramming medium comprises (i) potassium ion at a concentration higher than 5 mM and (ii) mannose. In some aspects, the metabolic reprogramming medium comprises (i) potassium ion at a concentration of at least about 30 mM to at least about 100 mM and (ii) mannose. In some aspects, the metabolic reprogramming medium comprises (i) potassium ion at a concentration of higher than 40 mM and (ii) mannose. In some aspects, the metabolic reprogramming medium comprises (i) potassium ion at a concentration of at least about 50 mM and (ii) mannose. In some aspects, the metabolic reprogramming medium is hypotonic. In some aspects, the medium is isotonic. In some aspects, the metabolic reprogramming medium comprises (i) potassium ion at a concentration higher than 40 mM and (ii) glucose; wherein the total concentration of potassium ion and NaCl is between 110 mM and 140 mM. In some aspects, the metabolic reprogramming medium comprises (i) potassium ion at a concentration higher than 50 mM and (ii) glucose; wherein the total concentration of potassium ion and NaCl is between 110 mM and 140 mM. In some aspects, the metabolic reprogramming medium comprises (i) potassium ion at a concentration of at least about 40 mM and (ii) mannose; wherein the total concentration of potassium ion and NaCl is between 110 mM and 140 mM. In some aspects, the metabolic reprogramming medium comprises (i) potassium ion at a concentration of at least about 50 mM and (ii) mannose; wherein the total concentration of potassium ion and NaCl is between 110 mM and 140 mM.

In some aspects, the concentration of the saccharide, e.g., glucose, is about 10 mM to about 24 mM. In some aspects, the concentration of the saccharide, e.g., glucose, is less than about 4.29 g/L. In some aspects, the concentration of the saccharide, e.g., glucose, is less than about 24 mM. In some aspects, the concentration of the saccharide, e.g., glucose, is more than about 5 mM. In some aspects, the concentration of the saccharide, e.g., glucose, is about 5 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 25 mM, about 10 mM to about 20 mM, about 10 mM to about 5 mM, about 15 mM to about 25 mM, about 15 mM to about 20 mM, about 15 mM to about 19 mM, about 15 mM to about 18 mM, about 15 mM to about 17 mM, about 15 mM to about 16 mM, about 16 mM to about 20 mM, about 16 mM to about 19 mM, about 16 mM to about 18 mM, about 16 mM to about 17 mM, about 17 mM to about 20 mM, about 17 mM to about 19 mM, or about 17 mM to about 18 mM. In some aspects, the concentration of the saccharide, e.g., glucose, is from about 5 mM to about 20 mM. In some aspects, the concentration of the saccharide, e.g., glucose, is from about 10 mM to about 20 mM. In some aspects, the concentration of the saccharide, e.g., glucose, is from about 10 mM to about 15 mM. In some aspects, the concentration of the saccharide, e.g., glucose, is from about 14 mM to about 14.5 mM. In some aspects, the concentration of the saccharide, e.g., glucose, is from about 14.5 mM to about 15 mM. In some aspects, the concentration of the saccharide, e.g., glucose, is from about 15 mM to about 15.5 mM. In some aspects, the concentration of the saccharide, e.g., glucose, is from about 15.5 mM to about 16 mM. In some aspects, the concentration of the saccharide, e.g., glucose, is from about 16 mM to about 16.5 mM. In some aspects, the concentration of the saccharide, e.g., glucose, is from about 16.5 mM to about 17 mM. In some aspects, the concentration of the saccharide, e.g., glucose, is from about 17 mM to about 17.5 mM. In some aspects, the concentration of the saccharide, e.g., glucose, is from about 17.5 mM to about 18 mM.

In some aspects, the concentration of the saccharide, e.g., glucose, is about 5 mM, about 6 mM, about 7 mM, about 8 mM, about 9 mM, about 10 mM, is about 10.5 mM, about 11 mM, about 11.5 mM, about 12 mM, about 12.5 mM, about 13 mM, about 13.5 mM, about 14 mM, about 14.5 mM, about 15 mM, about 15.5 mM, about 16 mM, about 16.5 mM, about 17 mM, about 17.5 mM, about 18 mM, about 18.5 mM, about 19 mM, about 19.5 mM, about 20 mM, about 20.5 mM, about 21 mM, about 22 mM, about 23 mM, about 24 mM, or about 25 mM.

II.A.5. Calcium

Some aspects of the present disclosure are directed to methods of culturing immune cells, e.g., T cells and/or NK cells, comprising contacting the immune cells with PCS in a medium comprising (i) potassium ion at a concentration of at least about 5 mM and (ii) calcium ion. In some aspects, the medium is hypotonic or isotonic.

In some aspects, the target concentration of calcium is reached by starting with a basal medium comprising a higher concentration of calcium ion, and diluting the solution to reach the target concentration of calcium ion. In some aspects, the target concentration of calcium is reached by raising the concentration of calcium ion by adding one or more calcium salts. Non-limiting examples of calcium salts include calcium bromide, calcium carbonate, calcium chloride, calcium cyanamide, calcium fluoride, calcium hydride, calcium hydroxide, calcium iodate, calcium iodide, calcium nitrate, calcium nitrite, calcium oxalate, calcium perchlorate tetrahydrate, calcium phosphate monobasic, calcium phosphate tribasic, calcium sulfate, calcium thiocyanate tetrahydrate, hydroxyapatite, or any combination thereof. In some aspects, the calcium salt comprises calcium chloride (CaCl₂)). In some aspects, the calcium salt comprises calcium gluconate.

In some aspects, the concentration of the calcium ion is less than that of the basal medium. In some aspects, the concentration of the calcium ion is greater than that of the basal medium. In some aspects, the concentration of calcium ion is more than about 0.4 mM. In some aspects, the concentration of calcium ion is less than about 2.8 mM. In some aspects, the concentration of calcium ion is less than about 2.5 mM. In some aspects, the concentration of calcium ion is less than about 2.0 mM. In some aspects, the concentration of calcium ion is less than about 1.9 mM. In some aspects, the concentration of calcium ion is less than about 1.8 mM. In some aspects, the concentration of calcium ion is less than about 1.7 mM. In some aspects, the concentration of calcium ion is less than about 1.6 mM. In some aspects, the concentration of calcium ion is less than about 1.5 mM. In some aspects, the concentration of calcium ion is less than about 1.4 mM. In some aspects, the concentration of calcium ion is less than about 1.3 mM. In some aspects, the concentration of calcium ion is less than about 1.2 mM. In some aspects, the concentration of calcium ion is less than about 1.1 mM. In some aspects, the concentration of calcium ion is less than about 1.0 mM.

In some aspects, the concentration of calcium ion is from about 0.4 mM to about 2.8 mM, about 0.4 mM to about 2.7 mM, about 0.4 mM to about 2.5 mM, about 0.5 mM to about 2.0 mM, about 1.0 mM to about 2.0 mM, about 1.1 mM to about 2.0 mM, about 1.2 mM to about 2.0 mM, about 1.3 mM to about 2.0 mM, about 1.4 mM to about 2.0 mM, about 1.5 mM to about 2.0 mM, about 1.6 mM to about 2.0 mM, about 1.7 mM to about 2.0 mM, about 1.8 mM to about 2.0 mM, about 0.8 to about 0.9 mM, about 0.8 to about 1.0 mM, about 0.8 to about 1.1 mM, about 0.8 to about 1.2 mM, about 0.8 to about 1.3 mM, about 0.8 to about 1.4 mM, about 0.8 to about 1.5 mM, about 0.8 to about 1.6 mM, about 0.8 to about 1.7 mM, about 0.8 to about 1.8 mM, about 0.9 to about 1.0 mM, about 0.9 to about 1.1 mM, about 0.9 to about 1.2 mM, about 0.9 to about 1.3 mM, about 0.9 to about 1.4 mM, about 0.9 to about 1.5 mM, about 0.9 to about 1.6 mM, about 0.9 to about 1.7 mM, about 0.9 to about 1.8 mM, about 1.0 to about 1.1 mM, about 1.0 to about 1.2 mM, about 1.0 to about 1.3 mM, about 1.0 to about 1.4 mM, about 1.0 to about 1.5 mM, about 1.0 to about 1.6 mM, about 1.0 to about 1.7 mM, about 1.0 to about 1.8 mM, about 1.1 to about 1.2 mM, about 1.1 to about 1.3 mM, about 1.1 to about 1.4 mM, about 1.1 to about 1.5 mM, about 1.1 to about 1.6 mM, about 1.1 to about 1.7 mM, about 1.1 to about 1.8 mM, about 1.2 to about 1.3 mM, about 1.2 to about 1.4 mM, about 1.2 to about 1.5 mM, about 1.2 to about 1.6 mM, about 1.2 to about 1.7 mM, about 1.2 to about 1.8 mM, about 1.3 to about 1.4 mM, about 1.3 to about 1.5 mM, about 1.3 to about 1.6 mM, about 1.3 to about 1.7 mM, about 1.3 to about 1.8 mM, about 1.4 to about 1.5 mM, about 1.4 to about 1.6 mM, about 1.4 to about 1.7 mM, about 1.4 to about 1.8 mM, about 1.5 to about 1.6 mM, about 1.5 to about 1.7 mM, about 1.5 to about 1.8 mM, about 1.6 to about 1.7 mM, about 1.6 to about 1.8 mM, or about 1.7 to about 1.8 mM.

In some aspects, the concentration of calcium ion is from about 0.8 mM to about 1.8 mM. In some aspects, the concentration of calcium ion is from about 0.9 mM to about 1.8 mM. In some aspects, the concentration of calcium ion is from about 1.0 mM to about 1.8 mM. In some aspects, the concentration of calcium ion is from about 1.1 mM to about 1.8 mM. In some aspects, the concentration of calcium ion is from about 1.2 mM to about 1.8 mM. In some aspects, the concentration of calcium ion is from about 0.8 mM to about 1.8 mM. In some aspects, the concentration of calcium ion is from about 0.8 mM to about 0.9 mM. In some aspects, the concentration of calcium ion is from about 0.9 mM to about 1.0 mM. In some aspects, the concentration of calcium ion is from about 1.0 mM to about 1.1 mM. In some aspects, the concentration of calcium ion is from about 1.1 mM to about 1.2 mM. In some aspects, the concentration of calcium ion is from about 1.2 mM to about 1.3 mM. In some aspects, the concentration of calcium ion is from about 1.3 mM to about 1.4 mM. In some aspects, the concentration of calcium ion is from about 1.4 mM to about 1.5 mM. In some aspects, the concentration of calcium ion is from about 1.5 mM to about 1.6 mM. In some aspects, the concentration of calcium ion is from about 1.7 mM to about 1.8 mM.

In some aspects, the concentration of calcium ion is about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM. In some aspects, the concentration of calcium ion is about 0.6 mM. In some aspects, the concentration of calcium ion is about 0.7 mM. In some aspects, the concentration of calcium ion is about 0.8 mM. In some aspects, the concentration of calcium ion is about 0.9 mM. In some aspects, the concentration of calcium ion is about 1.0 mM. In some aspects, the concentration of calcium ion is about 1.1 mM. In some aspects, the concentration of calcium ion is about 1.2 mM. In some aspects, the concentration of calcium ion is about 1.3 mM. In some aspects, the concentration of calcium ion is about 1.4 mM. In some aspects, the concentration of calcium ion is about 1.5 mM. In some aspects, the concentration of calcium ion is about 1.6 mM. In some aspects, the concentration of calcium ion is about 1.7 mM. In some aspects, the concentration of calcium ion is about 1.8 mM.

II.A.6. Cytokines

In some aspects, the metabolic reprogramming medium comprises a cytokine. In some aspects, the medium is hypotonic. In some aspects, the medium is isotonic. In some aspects, the medium is hypertonic. In some aspects, the cytokine is selected from IL-2, IL-7, IL-15, IL-21, and any combination thereof. In some aspects, the metabolic reprogramming medium does not comprise IL-2. In some aspects, the metabolic reprogramming medium comprises IL2 and IL21. In some aspects, the metabolic reprogramming medium comprises IL2, IL21, and IL15. In some aspects, the cytokine is linked to or associated with the PCS.

The cytokine can be added to the medium at any point. In some aspects, the cytokine is added to the medium before the immune cells, e.g., T cells and/or NK cells, are added to the medium. In some aspects, the immune cells, e.g., T cells and/or NK cells, are contacted with PCS and cultured in the medium comprising (i) potassium at a concentration disclosed herein, and (ii) a cytokine prior to cell engineering, e.g., prior to transduction with a construct encoding a ligand binding protein. In some aspects, the immune cells, e.g., T cells and/or NK cells, are contacted with PCS and cultured in the medium comprising (i) potassium at a concentration disclosed herein, and (ii) a cytokine during cell engineering, e.g., during transduction with a ligand binding protein. In some aspects, the immune cells, e.g., T cells and/or NK cells, are contacted with PCS and cultured in the medium comprising (i) potassium at a concentration disclosed herein, and (ii) a cytokine after cell engineering, e.g., after transduction with a construct encoding polypeptide ligand binding protein. In some aspects, the immune cells, e.g., T cells and/or NK cells, are contacted with PCS and cultured in the medium comprising (i) potassium at a concentration disclosed herein, and (ii) a cytokine throughout cell expansion. In some aspects, the immune cells, e.g., T cells and/or NK cells, are contacted with PCS and cultured in the medium comprising (i) potassium at a concentration disclosed herein, and (ii) a cytokine throughout cell engineering and cell expansion.

In some aspects, the metabolic reprogramming medium comprises (i) at least about 5 mM potassium ion and (ii) IL-2. In some aspects, the metabolic reprogramming medium comprises (i) more than 40 mM potassium ion and (ii) IL-2. In some aspects, the metabolic reprogramming medium comprises (i) at least about 50 mM potassium ion and (ii) IL-2. In some aspects, the metabolic reprogramming medium comprises (i) at least about 5 mM potassium ion and (ii) IL-7. In some aspects, the metabolic reprogramming medium comprises (i) more than 40 mM potassium ion and (ii) IL-7. In some aspects, the metabolic reprogramming medium comprises (i) at least about 50 mM potassium ion and (ii) IL-7. In some aspects, the metabolic reprogramming medium comprises (i) at least about 5 mM potassium ion and (ii) IL-15. In some aspects, the metabolic reprogramming medium comprises (i) more than 40 mM potassium ion and (ii) IL-15. In some aspects, the metabolic reprogramming medium comprises (i) at least about 50 mM potassium ion and (ii) IL-15. In some aspects, the metabolic reprogramming medium comprises (i) at least about 5 mM potassium ion and (ii) IL-21. In some aspects, the metabolic reprogramming medium comprises (i) more than 40 mM potassium ion and (ii) IL-21. In some aspects, the metabolic reprogramming medium comprises (i) at least about 50 mM potassium ion and (ii) IL-21. In some aspects, the metabolic reprogramming medium comprises (i) at least about 5 mM potassium ion and (ii) IL-2, and the metabolic reprogramming medium does not comprise IL-7. In some aspects, the metabolic reprogramming medium comprises (i) more than 40 mM potassium ion and (ii) IL-2, and the metabolic reprogramming medium does not comprise IL-7. In some aspects, the metabolic reprogramming medium comprises (i) at least about 50 mM potassium ion and (ii) IL-2, and the metabolic reprogramming medium does not comprise IL-7. In some aspects, the metabolic reprogramming medium comprises (i) at least about 5 mM potassium ion and (ii) IL-2, and the metabolic reprogramming medium does not comprise IL-15. In some aspects, the metabolic reprogramming medium comprises (i) more than 40 mM potassium ion and (ii) IL-2, and the metabolic reprogramming medium does not comprise IL-15. In some aspects, the metabolic reprogramming medium comprises (i) at least about 50 mM potassium ion and (ii) IL-2, and the metabolic reprogramming medium does not comprise IL-15. In some aspects, the metabolic reprogramming medium comprises (i) at least about 5 mM potassium ion and (ii) IL-2, and the metabolic reprogramming medium does not comprise IL-7 and IL-15. In some aspects, the metabolic reprogramming medium comprises (i) more than 40 mM potassium ion and (ii) IL-2, and the metabolic reprogramming medium does not comprise IL-7 and IL-15. In some aspects, the metabolic reprogramming medium comprises (i) at least about 50 mM potassium ion and (ii) IL-2, and the metabolic reprogramming medium does not comprise IL-7 and IL-15. In some aspects, the metabolic reprogramming medium comprises (i) at least about 5 mM potassium ion and (ii) IL-2 and IL-21. In some aspects, the metabolic reprogramming medium comprises (i) more than 40 mM potassium ion and (ii) IL-2 and IL-21. In some aspects, the metabolic reprogramming medium comprises (i) at least about 50 mM potassium ion and (ii) IL-2 and IL-21. In some aspects, the metabolic reprogramming medium comprises (i) at least about 5 mM potassium ion and (ii) IL-7 and IL-21. In some aspects, the metabolic reprogramming medium comprises (i) more than 40 mM potassium ion and (ii) IL-7 and IL-21. In some aspects, the metabolic reprogramming medium comprises (i) at least about 50 mM potassium ion and (ii) IL-7 and IL-21. In some aspects, the metabolic reprogramming medium comprises (i) at least about 5 mM potassium ion and (ii) IL-15 and IL-21. In some aspects, the metabolic reprogramming medium comprises (i) more than 40 mM potassium ion and (ii) IL-15 and IL-21. In some aspects, the metabolic reprogramming medium comprises (i) at least about 50 mM potassium ion and (ii) IL-15 and IL-21. In some aspects, the metabolic reprogramming medium is hypotonic. In some aspects, the metabolic reprogramming medium is isotonic. In some aspects, the metabolic reprogramming medium further comprises NaCl, wherein the total concentration of potassium ion and NaCl is from 110 mM to 140 mM.

In some aspects, the metabolic reprogramming medium described herein (e.g., comprising potassium ion at a concentration greater than 5 mM) comprises between about 50 IU/mL to about 500 IU/mL of IL-2. In some aspects, the metabolic reprogramming medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-2.

Therefore, in some aspects, the metabolic reprogramming medium comprises (i) potassium ion at a concentration higher than 5 mM and (ii) about 50 IU/mL of IL-2. In some aspects, the metabolic reprogramming medium comprises (i) potassium ion at a concentration higher than 5 mM and (ii) about 60 IU/mL of IL-2. In some aspects, the metabolic reprogramming medium comprises (i) potassium ion at a concentration higher than 5 mM and (ii) about 70 IU/mL of IL-2. In some aspects, the metabolic reprogramming medium comprises (i) potassium ion at a concentration higher than 5 mM and (ii) about 80 IU/mL of IL-2. In some aspects, the metabolic reprogramming medium comprises (i) potassium ion at a concentration higher than 5 mM and (ii) about 90 IU/mL of IL-2. In some aspects, the metabolic reprogramming medium comprises (i) potassium ion at a concentration higher than 5 mM and (ii) about 100 IU/mL of IL-2. In some aspects, the metabolic reprogramming medium comprises (i) potassium ion at a concentration higher than 5 mM and (ii) about 125 IU/mL of IL-2. In some aspects, the metabolic reprogramming medium comprises (i) potassium ion at a concentration higher than 5 mM and (ii) about 150 IU/mL of IL-2. In some aspects, the metabolic reprogramming medium comprises (i) potassium ion at a concentration higher than 5 mM and (ii) about 175 IU/mL of IL-2. In some aspects, the metabolic reprogramming medium comprises (i) potassium ion at a concentration higher than 5 mM and (ii) about 200 IU/mL of IL-2. In some aspects, the metabolic reprogramming medium comprises (i) potassium ion at a concentration higher than 5 mM and (ii) about 225 IU/mL of IL-2. In some aspects, the metabolic reprogramming medium comprises (i) potassium ion at a concentration higher than 5 mM and (ii) about 250 IU/mL of IL-2. In some aspects, the metabolic reprogramming medium comprises (i) potassium ion at a concentration higher than 5 mM and (ii) about 275 IU/mL of IL-2. In some aspects, the metabolic reprogramming medium comprises (i) potassium ion at a concentration higher than 5 mM and (ii) about 300 IU/mL of IL-2. In some aspects, the metabolic reprogramming medium comprises (i) potassium ion at a concentration higher than 5 mM and (ii) about 350 IU/mL of IL-2. In some aspects, the metabolic reprogramming medium comprises (i) potassium ion at a concentration higher than 5 mM and (ii) about 400 IU/mL of IL-2. In some aspects, the metabolic reprogramming medium comprises (i) potassium ion at a concentration higher than 5 mM and (ii) about 450 IU/mL of IL-2. In some aspects, the metabolic reprogramming medium comprises (i) potassium ion at a concentration higher than 5 mM and (ii) about 500 IU/mL of IL-2. In some aspects, the metabolic reprogramming medium comprising potassium ion and IL-2 further comprises NaCl at a concentration less than about 115 nM.

In some aspects, the metabolic reprogramming medium comprises at least about 0.1 ng/mL IL-2. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-2.

In some aspects, the metabolic reprogramming medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-2. In some aspects, the metabolic reprogramming medium comprises at least about 1.0 ng/mL IL-2. In some aspects, the metabolic reprogramming medium comprises at least about 2.0 ng/mL IL-2. In some aspects, the metabolic reprogramming medium comprises at least about 3.0 ng/mL IL-2. In some aspects, the metabolic reprogramming medium comprises at least about 4.0 ng/mL IL-2. In some aspects, the metabolic reprogramming medium comprises at least about 5.0 ng/mL IL-2. In some aspects, the metabolic reprogramming medium comprises at least about 6.0 ng/mL IL-2. In some aspects, the metabolic reprogramming medium comprises at least about 7.0 ng/mL IL-2. In some aspects, the metabolic reprogramming medium comprises at least about 8.0 ng/mL IL-2. In some aspects, the metabolic reprogramming medium comprises at least about 9.0 ng/mL IL-2. In some aspects, the metabolic reprogramming medium comprises at least about 10 ng/mL IL-2.

In some aspects, the metabolic reprogramming medium comprises at least about 0.1 ng/mL IL-2. In some aspects, the metabolic reprogramming medium comprises from about 50 ng/mL to about 600 ng/mL, about 50 ng/mL to about 500 ng/mL, about 50 ng/mL to about 450 ng/mL, about 50 ng/mL to about 400 ng/mL, about 50 ng/mL to about 350 ng/mL, about 50 ng/mL to about 300 ng/mL, about 100 ng/mL to about 600 ng/mL, about 100 ng/mL to about 500 ng/mL, about 100 ng/mL to about 450 ng/mL, about 100 ng/mL to about 400 ng/mL, about 100 ng/mL to about 350 ng/mL, about 100 ng/mL to about 300 ng/mL, about 200 ng/mL to about 500 ng/mL, about 200 ng/mL to about 450 ng/mL, about 200 ng/mL to about 400 ng/mL, about 200 ng/mL to about 350 ng/mL, about 200 ng/mL to about 300 ng/mL, about 250 ng/mL to about 350 ng/mL, about 300 ng/mL to about 600 ng/mL, about 300 ng/mL to about 500 ng/mL, about 300 ng/mL to about 450 ng/mL, about 300 ng/mL to about 400 ng/mL, about 300 ng/mL to about 350 ng/mL, about 250 ng/mL to about 300 ng/mL, or about 275 ng/mL to about 325 ng/mL IL-2.

In some aspects, the metabolic reprogramming medium comprises at least about 50 ng/mL, at least about 60 ng/mL, at least about 70 ng/mL, at least about 80 ng/mL, at least about 90 ng/mL, at least about 100 ng/mL, at least about 110 ng/mL, at least about 120 ng/mL, at least about 130 ng/mL, at least about 140 ng/mL, at least about 150 ng/mL, at least about 160 ng/mL, at least about 170 ng/mL, at least about 180 ng/mL, at least about 190 ng/mL, at least about 200 ng/mL, at least about 210 ng/mL, at least about 220 ng/mL, at least about 230 ng/mL, at least about 240 ng/mL, at least about 250 ng/mL, at least about 260 ng/mL, at least about 270 ng/mL, at least about 280 ng/mL, at least about 290 ng/mL, at least about 300 ng/mL, at least about 310 ng/mL, at least about 320 ng/mL, at least about 330 ng/mL, at least about 340 ng/mL, at least about 350 ng/mL, at least about 360 ng/mL, at least about 370 ng/mL, at least about 380 ng/mL, at least about 390 ng/mL, at least about 400 ng/mL, at least about 410 ng/mL, at least about 420 ng/mL, at least about 430 ng/mL, at least about 440 ng/mL, at least about 450 ng/mL, at least about 460 ng/mL, at least about 470 ng/mL, at least about 480 ng/mL, at least about 490 ng/mL, at least about 500 ng/mL, at least about 510 ng/mL, at least about 520 ng/mL, at least about 530 ng/mL, at least about 540 ng/mL, at least about 550 ng/mL, at least about 560 ng/mL, at least about 570 ng/mL, at least about 580 ng/mL, at least about 590 ng/mL, or at least about 600 ng/mL IL-2. In some aspects, the metabolic reprogramming medium comprises at least about 50 ng/mL IL-2. In some aspects, the metabolic reprogramming medium comprises at least about 60 ng/mL IL-2. In some aspects, the metabolic reprogramming medium comprises at least about 70 ng/mL IL-2. In some aspects, the metabolic reprogramming medium comprises at least about 73.6 ng/mL IL-2. In some aspects, the metabolic reprogramming medium comprises at least about 75 ng/mL IL-2. In some aspects, the metabolic reprogramming medium comprises at least about 80 ng/mL IL-2. In some aspects, the metabolic reprogramming medium comprises at least about 90 ng/mL IL-2. In some aspects, the metabolic reprogramming medium comprises at least about 100 ng/mL IL-2. In some aspects, the metabolic reprogramming medium comprises at least about 200 ng/mL IL-2. In some aspects, the metabolic reprogramming medium comprises at least about 300 ng/mL IL-2. In some aspects, the metabolic reprogramming medium comprises at least about 400 ng/mL IL-2. In some aspects, the metabolic reprogramming medium comprises at least about 500 ng/mL IL-2. In some aspects, the metabolic reprogramming medium comprises at least about 600 ng/mL IL-2.

In some aspects, the metabolic reprogramming medium described herein (e.g., comprising potassium ion at a concentration greater than 5 mM) comprises between about 50 IU/mL to about 500 IU/mL of IL-21. In some aspects, the culture medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-21.

In some aspects, the metabolic reprogramming medium comprises (i) potassium ion at a concentration higher than 5 mM and (ii) about 50 IU/mL of IL-21. In some aspects, the metabolic reprogramming medium comprises (i) potassium ion at a concentration higher than 5 mM and (ii) about 60 IU/mL of IL-21. In some aspects, the metabolic reprogramming medium comprises (i) potassium ion at a concentration higher than 5 mM and (ii) about 70 IU/mL of IL-21. In some aspects, the metabolic reprogramming medium comprises (i) potassium ion at a concentration higher than 5 mM and (ii) about 80 IU/mL of IL-21. In some aspects, the metabolic reprogramming medium comprises (i) potassium ion at a concentration higher than 5 mM and (ii) about 90 IU/mL of IL-21. In some aspects, the metabolic reprogramming medium comprises (i) potassium ion at a concentration higher than 5 mM and (ii) about 100 IU/mL of IL-21. In some aspects, the metabolic reprogramming medium comprises (i) potassium ion at a concentration higher than 5 mM and (ii) about 125 IU/mL of IL-21. In some aspects, the metabolic reprogramming medium comprises (i) potassium ion at a concentration higher than 5 mM and (ii) about 150 IU/mL of IL-21. In some aspects, the metabolic reprogramming medium comprises (i) potassium ion at a concentration higher than 5 mM and (ii) about 175 IU/mL of IL-21. In some aspects, the metabolic reprogramming medium comprises (i) potassium ion at a concentration higher than 5 mM and (ii) about 200 IU/mL of IL-21. In some aspects, the metabolic reprogramming medium comprises (i) potassium ion at a concentration higher than 5 mM and (ii) about 225 IU/mL of IL-21. In some aspects, the metabolic reprogramming medium comprises (i) potassium ion at a concentration higher than 5 mM and (ii) about 250 IU/mL of IL-21. In some aspects, the metabolic reprogramming medium comprises (i) potassium ion at a concentration higher than 5 mM and (ii) about 275 IU/mL of IL-21. In some aspects, the metabolic reprogramming medium comprises (i) potassium ion at a concentration higher than 5 mM and (ii) about 300 IU/mL of IL-21. In some aspects, the metabolic reprogramming medium comprises (i) potassium ion at a concentration higher than 5 mM and (ii) about 350 IU/mL of IL-21. In some aspects, the metabolic reprogramming medium comprises (i) potassium ion at a concentration higher than 5 mM and (ii) about 400 IU/mL of IL-21. In some aspects, the metabolic reprogramming medium comprises (i) potassium ion at a concentration higher than 5 mM and (ii) about 450 IU/mL of IL-21. In some aspects, the metabolic reprogramming medium comprises (i) potassium ion at a concentration higher than 5 mM and (ii) about 500 IU/mL of IL-21. In some aspects, the metabolic reprogramming medium comprising potassium ion and IL-21 further comprises NaCl at a concentration less than about 115 nM.

In some aspects, the metabolic reprogramming medium comprises at least about 0.1 ng/mL IL-21. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-21.

In some aspects, the metabolic reprogramming medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-21. In some aspects, the metabolic reprogramming medium comprises at least about 1.0 ng/mL IL-21. In some aspects, the metabolic reprogramming medium comprises at least about 2.0 ng/mL IL-21. In some aspects, the metabolic reprogramming medium comprises at least about 3.0 ng/mL IL-21. In some aspects, the metabolic reprogramming medium comprises at least about 4.0 ng/mL IL-21. In some aspects, the metabolic reprogramming medium comprises at least about 5.0 ng/mL IL-21. In some aspects, the metabolic reprogramming medium comprises at least about 6.0 ng/mL IL-21. In some aspects, the metabolic reprogramming medium comprises at least about 7.0 ng/mL IL-21. In some aspects, the metabolic reprogramming medium comprises at least about 8.0 ng/mL IL-21. In some aspects, the metabolic reprogramming medium comprises at least about 9.0 ng/mL IL-21. In some aspects, the metabolic reprogramming medium comprises at least about 10 ng/mL IL-21. In some aspects, the metabolic reprogramming medium comprises at least about 10 ng/mL IL-21. In some aspects, the metabolic reprogramming medium comprises at least about 15 ng/mL IL-21. In some aspects, the metabolic reprogramming medium comprises at least about 20 ng/mL IL-21. In some aspects, the metabolic reprogramming medium comprises at least about 25 ng/mL IL-21. In some aspects, the metabolic reprogramming medium comprises at least about 30 ng/mL IL-21. In some aspects, the metabolic reprogramming medium comprises at least about 35 ng/mL IL-21.

In some aspects, the metabolic reprogramming medium described herein (e.g., comprising potassium ion at a concentration greater than 5 mM) comprises between about 500 IU/mL to about 1,500 IU/mL of IL-7. In some aspects, the culture medium comprises about 500 IU/mL, about 550 IU/mL, about 600 IU/mL, about 650 IU/mL, about 700 IU/mL, about 750 IU/mL, about 800 IU/mL, about 850 IU/mL, about 900 IU/mL, about 950 IU/mL, about 1,000 IU/mL, about 1,050 IU/mL, about 1,100 IU/mL, about 1,150 IU/mL, about 1,200 IU/mL, about 1,250 IU/mL, about 1,300 IU/mL, about 1,350 IU/mL, about 1,400 IU/mL, about 1,450 IU/mL, or about 1,500 IU/mL of IL-7.

In some aspects, the metabolic reprogramming medium useful for the present disclosure comprises (i) potassium ion at a concentration higher than 5 mM and (ii) about 500 IU/mL of IL-7. In some aspects, the metabolic reprogramming medium comprises (i) potassium ion at a concentration higher than 5 mM and (ii) about 550 IU/mL of IL-7. In some aspects, the metabolic reprogramming medium comprises (i) potassium ion at a concentration higher than 5 mM and (ii) about 600 IU/mL of IL-7. In some aspects, the metabolic reprogramming medium comprises (i) potassium ion at a concentration higher than 5 mM and (ii) about 650 IU/mL of IL-7. In some aspects, the metabolic reprogramming medium comprises (i) potassium ion at a concentration higher than 5 mM and (ii) about 700 IU/mL of IL-7. In some aspects, the metabolic reprogramming medium comprises (i) potassium ion at a concentration higher than 5 mM and (ii) about 750 IU/mL of IL-7. In some aspects, the metabolic reprogramming medium comprises (i) potassium ion at a concentration higher than 5 mM and (ii) about 800 IU/mL of IL-7. In some aspects, the metabolic reprogramming medium comprises (i) potassium ion at a concentration higher than 5 mM and (ii) about 850 IU/mL of IL-7. In some aspects, the metabolic reprogramming medium comprises (i) potassium ion at a concentration higher than 5 mM and (ii) about 900 IU/mL of IL-7. In some aspects, the metabolic reprogramming medium comprises (i) potassium ion at a concentration higher than 5 mM and (ii) about 950 IU/mL of IL-7. In some aspects, the metabolic reprogramming medium comprises (i) potassium ion at a concentration higher than 5 mM and (ii) about 1,000 IU/mL of IL-7. In some aspects, the metabolic reprogramming medium comprises (i) potassium ion at a concentration higher than 5 mM and (ii) about 1,050 IU/mL of IL-7. In some aspects, the metabolic reprogramming medium comprises (i) potassium ion at a concentration higher than 5 mM and (ii) about 1,100 IU/mL of IL-7. In some aspects, the metabolic reprogramming medium comprises (i) potassium ion at a concentration higher than 5 mM and (ii) about 1,150 IU/mL of IL-7. In some aspects, the metabolic reprogramming medium comprises (i) potassium ion at a concentration higher than 5 mM and (ii) about 1,200 IU/mL of IL-7. In some aspects, the metabolic reprogramming medium comprises (i) potassium ion at a concentration higher than 5 mM and (ii) about 1,250 IU/mL of IL-7. In some aspects, the metabolic reprogramming medium comprises (i) potassium ion at a concentration higher than 5 mM and (ii) about 1,300 IU/mL of IL-7. In some aspects, the metabolic reprogramming medium comprises (i) potassium ion at a concentration higher than 5 mM and (ii) about 1,350 IU/mL of IL-7. In some aspects, the metabolic reprogramming medium comprises (i) potassium ion at a concentration higher than 5 mM and (ii) about 1,400 IU/mL of IL-7. In some aspects, the metabolic reprogramming medium comprises (i) potassium ion at a concentration higher than 5 mM and (ii) about 1,450 IU/mL of IL-7. In some aspects, the metabolic reprogramming medium comprises (i) potassium ion at a concentration higher than 5 mM and (ii) about 1,500 IU/mL of IL-7. In some aspects, the metabolic reprogramming medium comprising potassium ion and IL-7 further comprises NaCl at a concentration less than about 115 nM.

In some aspects, the metabolic reprogramming medium comprises at least about 0.1 ng/mL IL-7. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-7.

In some aspects, the metabolic reprogramming medium comprises at least about 0.1 ng/mL, at least about 0.5 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-7. In some aspects, the metabolic reprogramming medium comprises at least about 1.0 ng/mL IL-7. In some aspects, the metabolic reprogramming medium comprises at least about 2.0 ng/mL IL-7. In some aspects, the metabolic reprogramming medium comprises at least about 3.0 ng/mL IL-7. In some aspects, the metabolic reprogramming medium comprises at least about 4.0 ng/mL IL-7. In some aspects, the metabolic reprogramming medium comprises at least about 5.0 ng/mL IL-7. In some aspects, the metabolic reprogramming medium comprises at least about 6.0 ng/mL IL-7. In some aspects, the metabolic reprogramming medium comprises at least about 7.0 ng/mL IL-7. In some aspects, the metabolic reprogramming medium comprises at least about 8.0 ng/mL IL-7. In some aspects, the metabolic reprogramming medium comprises at least about 9.0 ng/mL IL-7. In some aspects, the metabolic reprogramming medium comprises at least about 10 ng/mL IL-7.

In some aspects, the metabolic reprogramming medium described herein (e.g., comprising potassium ion at a concentration greater than 5 mM) comprises between about 50 IU/mL to about 500 IU/mL of IL-15. In some aspects, the culture medium comprises about 50 IU/mL, about 60 IU/mL, about 70 IU/mL, about 80 IU/mL, about 90 IU/mL, about 100 IU/mL, about 125 IU/mL, about 150 IU/mL, about 175 IU/mL, about 200 IU/mL, about 225 IU/mL, about 250 IU/mL, about 275 IU/mL, about 300 IU/mL, about 350 IU/mL, about 400 IU/mL, about 450 IU/mL, or about 500 IU/mL of IL-15.

Therefore, in some aspects, the metabolic reprogramming medium comprises (i) potassium ion at a concentration higher than 5 mM and (ii) about 50 IU/mL of IL-15. In some aspects, the metabolic reprogramming medium comprises (i) potassium ion at a concentration higher than 5 mM and (ii) about 60 IU/mL of IL-15. In some aspects, the metabolic reprogramming medium comprises (i) potassium ion at a concentration higher than 5 mM and (ii) about 70 IU/mL of IL-15. In some aspects, the metabolic reprogramming medium comprises (i) potassium ion at a concentration higher than 5 mM and (ii) about 80 IU/mL of IL-15. In some aspects, the metabolic reprogramming medium comprises (i) potassium ion at a concentration higher than 5 mM and (ii) about 90 IU/mL of IL-15. In some aspects, the metabolic reprogramming medium comprises (i) potassium ion at a concentration higher than 5 mM and (ii) about 100 IU/mL of IL-15. In some aspects, the metabolic reprogramming medium comprises (i) potassium ion at a concentration higher than 5 mM and (ii) about 125 IU/mL of IL-15. In some aspects, the metabolic reprogramming medium comprises (i) potassium ion at a concentration higher than 5 mM and (ii) about 150 IU/mL of IL-15. In some aspects, the metabolic reprogramming medium comprises (i) potassium ion at a concentration higher than 5 mM and (ii) about 175 IU/mL of IL-15. In some aspects, the metabolic reprogramming medium comprises (i) potassium ion at a concentration higher than 5 mM and (ii) about 200 IU/mL of IL-15. In some aspects, the metabolic reprogramming medium comprises (i) potassium ion at a concentration higher than 5 mM and (ii) about 225 IU/mL of IL-15. In some aspects, the metabolic reprogramming medium comprises (i) potassium ion at a concentration higher than 5 mM and (ii) about 250 IU/mL of IL-15. In some aspects, the metabolic reprogramming medium comprises (i) potassium ion at a concentration higher than 5 mM and (ii) about 275 IU/mL of IL-15. In some aspects, the metabolic reprogramming medium comprises (i) potassium ion at a concentration higher than 5 mM and (ii) about 300 IU/mL of IL-15. In some aspects, the metabolic reprogramming medium comprises (i) potassium ion at a concentration higher than 5 mM and (ii) about 350 IU/mL of IL-15. In some aspects, the metabolic reprogramming medium comprises (i) potassium ion at a concentration higher than 5 mM and (ii) about 400 IU/mL of IL-15. In some aspects, the metabolic reprogramming medium comprises (i) potassium ion at a concentration higher than 5 mM and (ii) about 450 IU/mL of IL-15. In some aspects, the metabolic reprogramming medium comprises (i) potassium ion at a concentration higher than 5 mM and (ii) about 500 IU/mL of IL-15. In some aspects, the metabolic reprogramming medium comprising potassium ion and IL-15 further comprises NaCl at a concentration less than about 115 nM.

In some aspects, the metabolic reprogramming medium comprises at least about 0.1 ng/mL IL-15. In some aspects, the metabolic reprogramming medium comprises from about 0.1 ng/mL to about 20 ng/mL, about 1 ng/mL to about 20 ng/mL, about 1 ng/mL to about 15 ng/mL, about 1 ng/mL to about 14 ng/mL, about 1 ng/mL to about 13 ng/mL, about 1 ng/mL to about 12 ng/mL, about 1 ng/mL to about 11 ng/mL, about 1 ng/mL to about 10 ng/mL, about 1 ng/mL to about 9 ng/mL, about 1 ng/mL to about 8 ng/mL, about 1 ng/mL to about 7 ng/mL, about 1 ng/mL to about 6 ng/mL, about 1 ng/mL to about 5 ng/mL, about 1 ng/mL to about 4 ng/mL, about 1 ng/mL to about 3 ng/mL, about 1 ng/mL to about 2 ng/mL, about 5 ng/mL to about 15 ng/mL, about 5 ng/mL to about 10 ng/mL, about 10 ng/mL to about 20 ng/mL, about 10 ng/mL to about 15 ng/mL, or about 15 ng/mL to about 20 ng/mL IL-15.

In some aspects, the metabolic reprogramming medium comprises at least about 0.1 ng/mL, at least about 0.2 ng/mL, at least about 0.3 ng/mL, at least about 0.4 ng/mL, at least about 0.5 ng/mL, at least about 0.6 ng/mL, at least about 0.7 ng/mL, at least about 0.8 ng/mL, at least about 0.9 ng/mL, at least about 1 ng/mL, at least about 2 ng/mL, at least about 3 ng/mL, at least about 4 ng/mL, at least about 5 ng/mL, at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, at least about 16 ng/mL, at least about 17 ng/mL, at least about 18 ng/mL, at least about 19 ng/mL, or at least about 20 ng/mL IL-15. In some aspects, the metabolic reprogramming medium comprises at least about 1.0 ng/mL IL-15. In some aspects, the metabolic reprogramming medium comprises at least about 2.0 ng/mL IL-15. In some aspects, the metabolic reprogramming medium comprises at least about 3.0 ng/mL IL-15. In some aspects, the metabolic reprogramming medium comprises at least about 4.0 ng/mL IL-15. In some aspects, the metabolic reprogramming medium comprises at least about 5.0 ng/mL IL-15. In some aspects, the metabolic reprogramming medium comprises at least about 6.0 ng/mL IL-15. In some aspects, the metabolic reprogramming medium comprises at least about 7.0 ng/mL IL-15. In some aspects, the metabolic reprogramming medium comprises at least about 8.0 ng/mL IL-15. In some aspects, the metabolic reprogramming medium comprises at least about 9.0 ng/mL IL-15. In some aspects, the metabolic reprogramming medium comprises at least about 10 ng/mL IL-15. In some aspects, the metabolic reprogramming medium further comprises NaCl, wherein the total concentration of potassium ion and NaCl is from 110 mM to 140 mM.

In some aspects, the metabolic reprogramming medium comprises at least about 30 mM to at least about 100 mM potassium ion, about 300 ng/mL IL-2, and about 0.4 ng/mL IL-15. In some aspects, the metabolic reprogramming medium comprises more than 40 mM potassium ion, about 300 ng/mL IL-2, and about 0.4 ng/mL IL-15. In some aspects, the metabolic reprogramming medium comprises at least about 45 mM potassium ion, about 300 ng/mL IL-2, and about 0.4 ng/mL IL-15. In some aspects, the metabolic reprogramming medium comprises at least about 50 mM potassium ion, about 300 ng/mL IL-2, and about 0.4 ng/mL IL-15. In some aspects, the metabolic reprogramming medium comprises at least about 55 mM potassium ion, about 300 ng/mL IL-2, and about 0.4 ng/mL IL-15. In some aspects, the metabolic reprogramming medium comprises at least about 60 mM potassium ion, about 300 ng/mL IL-2, and about 0.4 ng/mL IL-15. In some aspects, the metabolic reprogramming medium comprises at least about 65 mM potassium ion, about 300 ng/mL IL-2, and about 0.4 ng/mL IL-15. In some aspects, the metabolic reprogramming medium comprises at least about 70 mM potassium ion, about 300 ng/mL IL-2, and about 0.4 ng/mL IL-15. In some aspects, the metabolic reprogramming medium comprises at least about 75 mM potassium ion, about 300 ng/mL IL-2, and about 0.4 ng/mL IL-15. In some aspects, the metabolic reprogramming medium comprises at least about 80 mM potassium ion, about 300 ng/mL IL-2, and about 0.4 ng/mL IL-15. In some aspects, the metabolic reprogramming medium comprises at least about 85 mM potassium ion, about 300 ng/mL IL-2, and about 0.4 ng/mL IL-15. In some aspects, the metabolic reprogramming medium comprises at least about 90 mM potassium ion, about 300 ng/mL IL-2, and about 0.4 ng/mL IL-15. In some aspects, the metabolic reprogramming medium comprises (i) at least about 70 mM potassium ion, (ii) about 60 mM NaCl, (iii) about 1.4 mM calcium, (iv) about 16 mM glucose, (v) about 300 ng/mL IL-2, and (vi) about 0.4 ng/mL IL-15.

II.A.7. Basal Media

In some aspects, the basal medium comprises a balanced salt solution (e.g., PBS, DPBS, HBSS, EBSS), Dulbecco's Modified Eagle's Medium (DMEM), Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ CTS™ T-Cell Expansion Basal Medium (ThermoFisher), OPTMIZER™ Complete, IMMUNOCULT™ XF (STEMCELL™ Technologies), IMMUNOCULT™, AIM V, TEXMACS™ medium, PRIME-XV® T cell CDM, X-VIVO™ 15 (Lonza), TRANSACT™ TIL expansion medium, or any combination thereof. In some aspects, the basal medium comprises PRIME-XV T cell CDM. In some aspects, the basal medium comprises OPTMIZER™. In some aspects, the basal medium comprises OPTMIZER™ Pro. In some aspects, the basal medium is serum free. In some aspects, the basal medium further comprises immune cell serum replacement (ICSR). For example, in some aspects, the basal medium comprises OPTMIZER™ Complete supplemented with ICSR, AIM V supplemented with ICSR, IMMUNOCULT™ XF supplemented with ICSR, RPMI supplemented with ICSR, TEXMACS™ supplemented with ICSR, or any combination thereof. In particular aspects, the basal medium comprises OPTMIZER™ complete.

In some aspects, the medium, e.g., the MRM, further comprises about 2.5% serum supplement (CTS™ Immune Cell SR, Thermo Fisher), 2 mM L-glutamine, 2 mM L-glutamax, MEM Non-Essential Amino Acids Solution, Pen-strep, 20 μg/ml Fungin™, sodium pyruvate, or any combination thereof. In some aspects, the medium further comprises O-Acetyl-L-carnitine hydrochloride. In some aspects, the medium further comprises a kinase inhibitor.

In some aspects, the medium further comprises a CD3 agonist. In some aspects, the CD3 agonist is an anti-CD3 antibody. In some aspects, the anti-CD3 antibody comprises OKT-3.

In some aspects, the medium further comprises a CD28 agonist. In some aspects, the CD28 agonist is an anti-CD28 antibody. In some aspects, the medium further comprises a CD27 ligand (CD27L). In some aspects, the medium further comprises a 4-1BB ligand (4-1BBL).

In some aspects, the present disclosure includes a cell culture comprising the medium disclosed herein, a cell bag comprising the medium disclosed herein, or a bioreactor comprising the medium disclosed herein.

II.B. Programmable Cell-Signaling Scaffolds (PCS)

In some aspects, the methods described herein comprise contacting human immune cells with programmable cell-signaling scaffolds (PCS) in a metabolic reprogramming medium (MRM), as described herein. Non-limiting examples of programmable cell-signaling scaffolds (PCS) are described in WO2018/013797 and Chung et al. (Nature Biotechnology 36(2): 160-169 (2018), the contents of which are incorporated by reference. In some aspects, the programmable cell-signaling scaffolds of the disclosure comprise a first layer comprising high surface area mesoporous silica micro rods (MSRs); a second layer comprising lipids coating said first layer; and a plurality of functional molecules loaded onto the scaffold. In some aspects, the scaffolds are biodegradable.

The scaffolds described herein are capable of mimicking functions commonly associated with antigen-presenting cells (APCs), which allows the scaffolds to elicit various functions on target cells, e.g., eliciting effector functions of T-cells. As contemplated herein, the scaffolds mediate these effects via either direct or indirect interactions between the cell surface molecules residing in target cells (e.g., T cells) and the various functional molecules presented by the scaffolds. In some aspects, the scaffold modulates survival of target cells (e.g., T cells), growth of targeted cells (e.g., T cells), and/or function of target cells (e.g., T cells) through the physical or chemical characteristics of a scaffold itself.

In some aspects, the scaffold composition is modified to comprise one or more surface cues and/or soluble cues (e.g., cell signaling molecules). In some aspects, the surface cues and/or soluble cues act to mediate various effector functions. Non-limiting examples of effector functions that can be affected by the surface cues and/or soluble cues include activation, division, promoting differentiation, growth, expansion, survival, increase yield, reprogramming, anergy, quiescence, senescence, apoptosis, death of target cells, or any combination thereof. In some aspects, the one or more surface cues and/or soluble cues act to increase “stemness.” In some aspects, cells, e.g., immune cells, contacted with the PCS described herein in the media described herein exhibit superior growth and function compared to cells, e.g., immune cells, contacted with other substrate platforms, such as magnetic beads, e.g., DYNABEADS™, or commercial particles, e.g., TRANSACT™ (Miltenyi Biotech).

In some aspects, the methods described herein comprise contacting human immune cells with PCS in a metabolic reprogramming medium, and further contacting the immune cells with one or more stimulatory molecules, cytokines, and/or other co-factors. In some aspects, the one or more stimulatory molecules, cytokines, and/or other co-factors are present in the medium. In some aspects, the one or more stimulatory molecules, cytokines, and/or other co-factors are present in the scaffold. In some aspects, non-targeted cells (e.g., cells other than T cells), which have otherwise infiltrated a scaffold, are rejected or removed using negative selection agents, cues, or through passive non-stimulation.

### Components of PCS

In some aspects, the specific components of a scaffold are modulated. The permeability of a scaffold composition can be regulated, for example, by selecting or engineering a material for greater or smaller pore size, density, polymer cross-linking, stiffness, toughness, ductility, or elasticity. A scaffold composition can contain physical channels or paths through which targeted cells interact with a scaffold and/or move into a specific compartment or region of a scaffold. As needed, to facilitate compartmentalization, a scaffold composition can be optionally organized into compartments or layers, each with a different permeability, so that cells can be sorted or filtered to allow access to only a certain sub-population of cells. Sequestration of target cell populations in the scaffold can also be regulated by the degradation, dehydration, re-hydration, oxygenation, chemical alteration, pH alteration, ongoing self-assembly of the scaffold composition, or any combination thereof. Further, the functional molecules of a scaffold can vary in type and relative abundance to elicit specific interactions with desired cells.

In some aspects, the PCS comprises (i) a base layer comprising high surface area mesoporous silica micro-rods (MSR); (ii) a continuous, fluid-supported lipid bilayer (SLB) layered on the MSR base layer; (iii) a plurality of surface cues loaded onto the scaffold; and/or (iv) a plurality of soluble cues loaded onto the scaffold.

II.B.1. Mesoporous Silica

In some aspects, the scaffold comprises mesoporous silica. Mesoporous silica is a porous body with hexagonal close-packed, cylinder-shaped, uniform pores. In some aspects, the mesoporous silica is synthesized by using a rod-like micelle of a surfactant as a template, which is formed in water by dissolving and hydrolyzing a silica source such as alkoxysilane, sodium silicate solution, kanemite, silica fine particle in water or alcohol in the presence of acid or basic catalyst. See, US Pub. No. 2015-0072009 and Hoffmann et al., Angewandte Chemie International Edition, 45, 3216-3251, 2006, each of which is incorporated by reference herein in its entirety. Many kinds of surfactants can be used in the synthesis of the mesoporous silica, including, but not limited to, cationic, anionic, and nonionic surfactants. In some aspects, the surfactant is an alkyl trimethylammonium salt of cationic surfactant. An alkyl trimethylammonium salt of cationic surfactant can yield a mesoporous silica having the increased specific surface area and pore volume. See U.S. Publication No. 2013/0052117 and Katiyar et al. (Journal of Chromatography 1122 (1-2): 13-20), each of which is incorporated by reference herein in its entirety. The terms “mesoscale,” “mesopore,” “mesoporous” and the like, as used herein, refer to structures having feature sizes in the range of about 1 nm to about 60 nm. In some aspects, the mesoporous material includes pores having a diameter in the range of about 1 nm to about 50 nm. In some aspects, the mesoporous material includes pores having a diameter in the range of about 5 nm to about 60 nm. In some aspects, the mesoporous material includes pores having a diameter in the range of about 2 nm to about 50 nm. In some aspects, the pores are orderly distributed. In some aspects, the pores are randomly distributed.

The mesoporous silica used in scaffolds of the disclosure can be provided in various forms. In some aspects, the scaffolds are provided in a form selected from microspheres, irregular particles, rectangular rods, round nanorods, and any combination thereof. In some aspects, the scaffolds are provided as structured rod-shaped forms (MSR). The particles can have any pre-determined shape. In some aspects, the particles have a spheroid shape. In some aspects, the particles have an ellipsoid shape. In some aspects, the particles have a rod-like shape. In some aspects, the particles have a curved cylindrical shape. Non-limiting examples of methods of assembling mesoporous silica to generate microrods can be found, e.g., in Wang et al, Journal of Nanoparticle Research, 15:1501, 2013, which is incorporated by reference herein in its entirety. In some aspects, mesoporous silica nanoparticles are synthesized by reacting tetraethyl orthosilicate with a template made of micellar rods. The template can then be removed by washing with a solvent adjusted to the proper pH. In this example, after removal of surfactant templates, hydrophilic silica nanoparticles characterized by a uniform, ordered, and connected mesoporosity are prepared with a specific surface area of, for example, about 600 m²/g to about 1200 m²/g, particularly about 800 m²/g to about 1000 m²/g and especially about 850 m²/g to about 950 m²/g. In some aspects, the mesoporous particle is synthesized using a simple sol-gel method or a spray drying method. Tetraethyl orthosilicate can also be used with an additional polymer monomer (e.g., as a template). In some aspects, one or more tetraalkoxy-silanes and one or more (3-cyanopropyl)trialkoxy-silanes are co-condensed to provide the mesoporous silicate particles as rods. See US Publication Nos. 2013-0145488, 2012-0264599 and 2012-0256336, each of which is hereby incorporated by reference in its entirety.

The MSR can comprise pores of between 1-60 nm in diameter, e.g., pores of between 2-5 nm, 10-20 nm, 10-30 nm, 10-40 nm, 20-30 nm, 30-50 nm, 30-40 nm, 40-50 nm, 50-60 nm. In some aspects, the microrods comprise pores of approximately 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, or more in diameter. The pore size can be altered depending on the type of application.

In some aspects, the length of the MSR is in the micrometer range, ranging from about 5 μm to about 500 μm. In some aspects, the microrods comprise a length of about 5-50 μm, e.g., about 10-20 μm, about 10-30 μm, about 10-40 μm, about 20-30 μm, about 30-50 μm, about 30-40 μm, or about 40-50 μm. In some aspects, the MSR comprise a length of about 50 μm to about 250 μm, e.g., about 60 μm, about 70 μm, about 80 μm, about 90 μm, about 100 μm, about 120 μm, about 150 μm, about 180 μm, about 200 μm, about 225 μm, or more. For recruitment of cells, MSR compositions having a higher aspect ratio can be employed, e.g., with rods comprising a length of 50 μm to 200 μm, particularly a length of 80 μm to 120 μm, especially a length of about 100 μm or more.

In some aspects, the width of the MSR is in the micrometer range, ranging from about 0.1 μm to about 100 μm. In some aspects, the microrods comprise a width of about 0.1-75 μm, e.g., about 1-55 μm, about 1-50 μm, about 2-50 μm, about 1-40 μm. In some aspects, the MSR comprise a width of about 1.0 μm, about 2 μm, about 5 μm, about 10 μm, about 15 μm, about 20 μm, about 25 μm, about 30 μm, about 35 μm, about 40 μm, about 45 μm, about 50 μm, about 55 μm or more.

In some aspects, the MSR provides a high surface area for attachment and/or binding to target cells, e.g., T-cells. Non-limiting methods of obtaining high surface area mesoporous silicates can be found, for example, in U.S. Pat. No. 8,883,308 and US Publication No. 2011-0253643, each of which is incorporated by reference herein in its entirety. In some aspects, the high surface area is due to the fibrous morphology of the nanoparticles, which makes it possible to obtain a high concentration of highly dispersed and easily accessible moieties on the surface. In some aspects, the MSR has a surface area of at least about 100 m²/g, at least 150 m²/g, at least about 200 m²/g, at least about 250 m²/g or at least 300 m²/g. In some aspects, the MSR has a surface area from about 100 m²/g to about 1500 m²/g, including all values or sub-ranges in between, e.g., 50 m²/g, 100 m²/g, 200 m²/g, 300 m²/g, 400 m²/g, 500 m²/g, 600 m²/g, 700 m²/g, 800 m²/g, 100-500 m²/g, 100-300 m²/g, 500-800 m²/g, 100-700 m²/g, 200-600 m²/g, 500-1000 m²/g or 500-1500 m²/g.

In some aspects, the MSR is sufficiently porous such that scaffolds sustain antigen presentation and attract and manipulate immune cells. In some aspects, scaffolds contain porous matrices, wherein the pores have a diameter of at least 10 nm. In some aspects, the pores have a diameter of at least 500 μm. In some aspects, the pores have a diameter from 10 nm to 500 μm. In some aspects, the pores have a diameter from 100 nm to 100 μm. In these aspects, the scaffolds comprise mesoporous scaffolds. In some aspects, scaffolds contain porous matrices, wherein the pores are as large or larger than the cell population infiltrating the scaffold. Non-limiting examples of methods of making polymer matrices having desired pore sizes and pore alignments are described, e.g., in US pub. No. 2011/0020216 and U.S. Pat. No. 6,511,650, each of which is incorporated herein by reference in its entirety.

II.B.2. Lipids

The scaffolds of the disclosure comprise a second layer comprising lipids coating said first layer. The term “lipid” generally denotes a heterogeneous group of substances associated with living systems which have the common property of being insoluble in water, can be extracted from cells by organic solvents of low polarity such as chloroform and ether. In some aspects, “lipid” refers to any substance that comprises long, fatty-acid chains, preferably containing 10-30 carbon units, particularly containing 14-23 carbon units, especially containing 16-18 carbon units.

In some aspects, the layer comprising lipids is provided as a monolayer. In some aspects, the layer comprising lipids is provided as a bilayer. Preferably, the lipid bilayer is fluid, wherein individual lipid molecules are able to diffuse within the bilayer. The membrane lipid molecules are preferably amphipathic.

In some aspects, the layer comprising lipids comprises one or more continuous bilayers, e.g., resembling those found in natural biological membranes such as cellular plasma membranes. In some aspects, the layer comprising lipids is provided in the form of a supported bilayer. In some aspects, the layer comprising lipids is a continuous, fluid-supported liposome. In some aspects, the layer comprising lipids is a continuous, fluid-supported lipid bilayer. As used herein, a supported bilayer is a planar structure sitting on a solid support. In such an arrangement, the upper face of the supported bilayer is exposed, while the inner face of the supported bilayer is in contact with the support. The scaffolds of the disclosure generally are stable and remain largely intact even when subject to high flow rates or vibration. The layer comprising lipids of scaffolds of the disclosure are also amenable to modification, derivatization, and/or chemical conjugation with any chemical and/or biological moiety.

In some aspects, the layer comprising lipids of scaffolds of the disclosure is immobilized on the MSR layer. The lipid layer can be immobilized on the MSR using any method, including, but not limited to, covalent and non-covalent interactions. In some aspects, the layer comprising lipids is adsorbed on the MSR layer. In some aspects, the layer comprising lipids is attached or tethered to the MSR via one or more covalent interactions. Non-limiting examples of methods for attaching lipids to silicates include surface absorption and physical immobilization, e.g., using a phase change to entrap the substance in the scaffold material. In some aspects, the layer comprising lipids is layered onto the MSR layer. For example, a lipid film (containing for example, a solution of DPPC/cholesterol/DSPE-PEG at a molar ratio of 77.5:20:2.5 in chloroform) can be spotted onto the MSR layer and the solvent is evaporated using a rotary evaporator. See Meng et al, ACS Nano, 9 (4), 3540-3557, 2015. In some aspects, the lipid bilayer is prepared by extrusion of hydrated lipid films through a filter with pore size of, e.g., about 100 nm. The filtered lipid films can then be fused with the porous particle cores, for example, by a pipette mixing.

In some aspects, covalent coupling via alkylating or acylating agents are used to provide a stable, structured, and long-term retention of the layer comprising lipids on the MSR layer. In some aspects, the lipid bilayers are reversibly or irreversibly immobilized onto the MSR layer. For example, the MSR layer can be hydrophilic and can be further treated to provide a more hydrophilic surface, e.g., with ammonium hydroxide and hydrogen peroxide. The lipid bilayer can be fused, e.g., using any coupling technique, onto the porous MSR layer to form scaffolds of the disclosure.

In some aspects, the layer comprising lipids comprises a phospholipid. Representative examples of such lipids include, but are not limited to, amphoteric liposomes described in U.S. Pat. Nos. 9,066,867 and 8,3676,28, each of which is incorporated by reference herein in its entirety. In some aspects, the layer comprising lipids comprises a lipid selected from dimyristoylphosphatidylcholine (DMPC), dipalmitoylphosphatidylcholine (DPPC), distearoylphosphatidylcholine (DSPC), palmitoyl-oleoylphosphatidylcholine (POPC), dioleoylphosphatidylcholine (DOPC), dioleoyl-phosphatidylethanolamine (DOPE), dimyristoyl-phosphatidylethanolamine (DMPE), dipalmitoyl-phosphatidylethanolamine (DPPE), 1-stearoyl-2-myristoyl-sn-glycero-3-phosphocholine (8:0-14:0 PC) and any combination thereof. In some aspects, the layer comprising lipids comprises palmitoyl-oleoylphosphatidylcholine (POPC). In some aspects, the layer comprising lipids comprises a lipid composition that mimics the lipid composition of a mammalian cell membrane (e.g., a human cell plasma membrane). The lipid compositions of many mammalian cell membranes have been characterized and are readily ascertainable by one of skill in the art (see, e.g., Essaid et al. Biochim. Biophys. Acta 1858(11): 2725-36 (2016), the entire contents of which are incorporated herein by reference). The composition of the layer comprising lipids can be altered to modify the charge or fluidity of the lipid bilayer. In some aspects, the layer comprising lipids comprises cholesterol. In some aspects, the layer comprising lipids comprises a sphingolipid. In some aspects, the layer comprising lipids comprises a phospholipid. In some aspects, the lipid is a phosphatidylethanolamine, a phosphatidylcholine, a phosphatidylserine, a phosphoinositide a phosphosphingolipid with saturated or unsaturated tails comprising 6-20 carbons, or a combination thereof. In some aspects, the lipid is a DIYNE PC lipid. In some aspects, the layer comprising lipids comprises a lipid composition that favors the spontaneous partitioning of lipid species into liquid-ordered domains (see, e.g., Wang T-Y et al. Biochemistry 40(43): 1303 1-40 (2001), which is incorporated by reference herein in its entirety).

In some aspects, the layer comprising lipids is stabilized by compounds such as ionic or non-ionic surfactants. Non-limiting examples of surfactants useful in the compositions disclosed herein include: synthetic phospholipids, their hydrogenated derivatives and mixtures thereof; sphingolipids and glycosphingolipids; saturated or unsaturated fatty acids; fatty alcohols; polyoxyethylene-polyoxypropylene copolymers; ethoxylated fatty acids as well as esters or ethers thereof, dimyristoyl phosphatidyl choline; dimyristoyl phosphatidyl glycerol; or a combination thereof. In some aspects, the surfactant comprises dimyristoyl phosphatidyl glycerol.

In some aspects, once in contact with a cell, a scaffold of the disclosure retains a continuous, fluid architecture for at least 1 day, at least 2 days, at least 3 days, at least 4 days, at least 5 days, at least 6 days, at least 7 days, at least 8 days, at least 9 days, at least 10, at least 11 days, at least 12 days, at least 13 days, at least 14 days, at least 15 days, at least 16 days, at least 17 days, at least 18 days, at least 19 days, at least 20 days, at least 2 1 days, at least 25 days, at least 30 days, at least 35 days, at least 40 days, at least 50 days, or more.

II.B.3. Biodegradable Scaffolds

In some aspects, the scaffolds of the disclosure are biodegradable. In some aspects, the scaffold structure substantially degrades when exposed to a biological milieu. In some aspects, the biological milieu comprises a tissue culture condition, e.g., tissue culture media that has been optionally adapted to culture lymphocytes such as T cells. In some aspects, the biological milieu comprises a biological fluid, e.g., blood, lymph, CSF, peritoneal fluid, or the like. In some aspects, the biological milieu is the tissue environment at the site of implant, e.g., blood vessels, lymphatic system, adipose tissue, or the like.

In some aspects, the biodegradable scaffolds are substantially degraded following contact with a biological milieu in vivo over 1 day, 2 days, 3 days, 4 days, 5 days, 6 days, 7 days, 8 days, 9 days, 10 days, 11 days, 12 days, 13 days, 14 days, 15 days, 20 days, 30 days, 45 days, 60 days, 90 days, or more. In some aspects, the biodegradable scaffolds are substantially degraded following contact with a biological milieu in vivo in less than 1 week. In some aspects, the biodegradable scaffolds are substantially degraded following contact with a biological milieu in vitro over 1 day, 2 days, 3 days, 4 days, 5 days, 6 days, 7, days, 8 days, 9 days, 10 days, 1 1 days, 12 days, 13 days, 14 days, 15 days, 20 days, 30 days, 45 days, 60 days, 90 days, or more. In some aspects, the biodegradable scaffolds are substantially degraded following contact with a biological milieu in vitro in less than 1 week. As used herein, “substantial degradation” means that at least 30%, at least 50%, at least 60%, at least 70%, at least 90%, at least 95%, or more of a scaffold composition is degraded when a scaffold composition is contacted with the biological milieu.

Accordingly, in some aspects, it is advantageous to tailor the degradation kinetics of a scaffold composition by modifying the properties of mesoporous silica rods, such as size, geometry, and/or porosity. Alternately, the degradation kinetics of a scaffold compositions can be modified by changing the culture conditions (e.g., by adjusting the pH of the media).

In accordance with the aforementioned aspects, a scaffold of the disclosure can comprise a plurality of functional molecules which are optionally biodegradable. In some aspects, the scaffolds of the instant disclosure are encapsulated into other biodegradable scaffolds. Non-limiting examples of reagents and techniques useful in making such composite biodegradable scaffold compositions are described in Liao et al, J. Biomed. Mater. Res. B. Appl. Biomater, 102(2):293-302, 2014, which is incorporated by reference herein its entirety. In some aspects, the scaffolds are made up of physiologically-compatible and optionally biodegradable polymers. Non-limiting examples of polymers that are employable in the scaffolds are described in U.S. Pat. No. 6,642,363, U.S. Publication No. 2011/0020216, Martinsen et al., Biotech. & Bioeng., 33 (1989) 79-89), (Matthew et al. Biomateriah, 16 (1995) 265-274), Atala et al., J Urology, 152 (1994) 641-643), and Smidsrod, TIBTECH 8 (1990) 71-78), the entire contents of which are incorporated herein by reference.

Aspects described herein further relate to programmable cell signaling scaffolds with one or more functional molecules, e.g., surface cues and soluble cues, optionally together with one or more additional agents. In some aspects, the disclosure provides compositions comprising a scaffold and T cells clustered therein. In some aspects, the compositions and/or scaffolds are provided with one or more reagents for selecting, culturing, expanding, sustaining, and/or transplanting the cells of interest.

II.B.4. Functional Molecules

In some aspects, the scaffolds comprise one or more functional molecules. In some aspects, the functional molecule interacts with cells, e.g., T cells, to elicit interaction and/or provoke or inhibit a response. In some aspects, the functional molecule is a surface cue. In some aspects, the functional molecule is a soluble cue. In some aspects, a scaffold comprises at least one surface cue. In some aspects, a scaffold comprises at least one soluble cue. In some aspects, a scaffold comprises at least one surface cue and at least one soluble cue.

Non-limiting examples of such functional molecules include polypeptides, antigens, antibodies, DNA, RNA, carbohydrates, haptens, other small molecules, and any combination thereof. In some aspects, the functional molecules of the disclosure comprise a polypeptide (used interchangeably herein with protein and peptide).

II.B.5. Surface Cues

In some aspects, the scaffolds comprise one or more surface cues. As used herein “surface cue” refers to molecules capable of binding to a cell surface receptor. In some aspects, the surface cue is in contact with, or coupled to, the layer comprising lipids of the scaffold structure. In some aspects, the surface cue mediates direct, indirect, or semi-direct modulation of one or more biological activities of a target population of cells, e.g., T cells. In some aspects, the surface cue mediates direct activation of T cells. In some aspects, the surface cue directly activates T-cells, e.g., via binding to cell surface receptors on target T-cells. In some aspects, the surface cue comprises a stimulatory molecule that is an activation signal to T cells. As used herein, a T cell “stimulatory molecule” refers to any agent that increases one or more T cell activity, increases the expression of one or more cytokine by the T cell, increases the cytotoxicity of the T cell, increases T cell proliferation, reduces T cell death, or any combination thereof. In some aspects, the surface cue comprises a co-stimulatory molecule.

In some aspects, the surface cue of a scaffold of the disclosure is an antibody or an antigen-binding portion thereof. The term “antibody,” as used herein, broadly refers to any immunoglobulin (Ig) molecule comprising one or more polypeptide chains. In some aspects, the antibody comprises two heavy (H) chains and two light (L) chains, or any functional fragment, mutant, variant, or derivation thereof, which retains the essential epitope binding features of an Ig molecule. As used herein, “antibody fragments” refer to a portion of an antibody, which is capable of binding an epitope on an antigen. The term “antigen-binding portion” of an antibody, as used herein, refers one or more part of an antibody that facilitates recognition of and/or binding to an antigen.

Non-limiting examples of antigen-binding portions within the scope of the present disclosure include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb fragment; and (vi) an isolated complementarity determining region (CDR). In some aspects, the antibody comprises a VHH antibody, a vNAR antibody, an IgNAR antibody, a camelid antibody, a diabody, a monobody, or any combination thereof.

In some aspects of the surface cues of the disclosure, the antibody is monospecific, bispecific, dual specific, or multi-specific formats; specifically binding to one, or two or more different, antigens.

In some aspects, the surface cues include, but are not limited to, a stimulatory molecule that activates T cells (T cell activating molecules). In some aspects, a stimulatory molecule activates T cells by engaging and/or clustering components of the T cell receptor complex. In some aspects, the stimulatory molecule comprises an anti-CD3 antibody or antigen-binding portion thereof. In some aspects, the stimulatory molecule comprises an anti-CD2 antibody or an antigen-binding portion thereof. In some aspects, the stimulatory molecule comprises an anti-CD47 antibody or an antigen-binding portion thereof. In some aspects, the stimulatory molecule comprises an anti-CD81 antibody or antigen-binding portion thereof. In some aspects, the stimulatory molecule comprises an anti-macrophage scavenger receptor (MSR1) antibody or an antigen-binding portion thereof. In some aspects, the stimulatory molecule comprises an anti-T-cell receptor (TCR) antibody or an antigen-binding portion thereof. In some aspects, the surface cue comprises a major histocompatibility complex (MHC) molecule or a multimer thereof. In some aspects, the major histocompatibility complex (MHC) molecule or a multimer thereof is loaded with an MHC peptide. In some aspects, the surface cue comprises a conjugate containing MHC and immunoglobulin (Ig) or a multimer thereof.

T cells can be activated in a CD3-dependent or independent manner, for example, via binding and/or ligation of CD3 or one or more cell-surface receptors other than CD3. Representative examples of such CD3-independent cell-surface molecules include, e.g., CD2, CD47, CD81, MSR1, etc. The process of T cell activation is characterized, for example, in Ryan et al, Nature Reviews Immunology 10, 7, 2010, which is incorporated by reference in its entirety.

In some aspects, the surface cue used in a scaffold of the disclosure is an anti-CD3 antibody or antigen-binding portion thereof. Representative examples of anti-CD3 antibodies include, but are not limited to, muromonab (OKT3), otelixizumab (TRX4), teplizumab (hOKT3yl(Ala-Ala)), visilizumab, an antibody recognizing 17-19 kD C-chain of CD3 within the CD3 antigen/T cell antigen receptor (TCR) complex (HIT3a), and an antibody recognizing a 20 kDa subunit of the TCR complex within CD3e (UCHT1), or an antigen-binding portion thereof. Additional non-limiting examples of anti-CD3 antibodies and antigen-binding portions thereof are described in US patent pub. No. 2014-0088295, which is incorporated herein by reference herein in its entirety.

In some aspects, the surface cue used in a scaffold of the disclosure comprises an anti-CD2 antibody or antigen-binding portion thereof. Representative examples of anti-CD2 antibodies include, but are not limited to, siplizumab (MEDI-507) and LO-CD2b, or an antigen-binding portion thereof. See, e.g., ATCC accession No. PTA-802; deposited Jun. 22, 1999.

In some aspects, the surface cue used in a scaffold of the disclosure comprises an anti-CD47 antibody or antigen-binding portion thereof. Representative examples of anti-CD47 antibodies include, but are not limited to, monoclonal antibody Hu5F9-G4, monoclonal antibody MABL-1, and monoclonal antibody MABL-2 (FERM Deposit Nos. BP-6100 and BP-6101), or an antigen-binding portion thereof. See, e.g., WO1999/1 2973, the disclosure in which is incorporated by reference herein.

In some aspects, the surface cue used in a scaffold of the disclosure comprises an anti-CD81 antibody or antigen-binding portion thereof. Representative examples of anti-CD81 antibodies include, but are not limited to, monoclonal antibody 5A6, or an antigen-binding portion thereof. See, e.g., Maecker et al., BMC Immunol., 4:1, 2003, the disclosure in which is incorporated by reference herein.

In some aspects, the surface cue used in a scaffold of the disclosure comprises an anti-MSRI antibody or antigen-binding portion thereof. Representative examples of anti-MSRI antibodies include, but are not limited to, rat anti-human CD204 antibody (Thermo Catalog No. MA5-16494) and goat anti-human CD204/MSR1 antibody (Biorad Catalog No. AHP563), or an antigen-binding portion thereof.

In some aspects, the surface cue used in a scaffold of the disclosure comprises an anti-TCR antibody or antigen-binding portion thereof. Representative examples of anti-TCR antibodies include, but are not limited to, mouse anti-human TCR monoclonal antibody IMMU510 (Immunotech, Beckman Coulter, Fullerton, Calif.) (described in Zhou et a, Cell Mol Immunol., 9(1): 34-44, 2012) and monoclonal antibody defining alpha/beta TCR WT31 (described in Gupta et al, Cell Immunol, 132(1):26-44, 1991), or an antigen-binding portion thereof.

In some aspects, the surface cue comprises a bispecific antibody. In some aspects, a bispecific antibody is used to bring a cell of interest, e.g., a cancer cell or a pathogen, in close proximity with a target effector cell of the disclosure, e.g., a cytotoxic T-cell, such that the effector function of the target effector cell is mediated specifically upon the cell of interest. In some aspects, the surface cue comprises a bispecific antibody, wherein one arm of the antibody is specific to a T cell antigen and the other arm of the antibody is specific to a tumor-associated antigen or a pathogen-specific antigen or mutants thereof.

In some aspects, a bispecific antibody functions in an activation and co-stimulatory capacity. In some aspects, the bispecific antibody specifically binds CD3 and CD28. Such surface cues can be referred to herein as, e.g., “anti-CD3/anti-CD28,” or “anti-CD3×CD28” or “CD3×CD28” bispecific molecules, or other similar terminology. The human CD28 protein has the amino acid sequence shown in GENBANK accession Nos. NP_001230006.1, NP_001230007.1, or NP_006130.1. The mouse CD28 protein has the amino acid sequence shown in GENBANK accession No. NP_031668.3. The various polypeptide sequences encompassed by the aforementioned accession numbers, include, the corresponding mRNA and gene sequences, and are incorporated by reference herein in their entirety. Additional examples of bispecific antibodies envisaged within the scope of the instant disclosure include, but are not limited to, solitomab (CD3×EpCAM), blinatumomab (CD3×CD19), MAB MT-111 (CD3×CEA), and BAY-2010112 (CD3×PSMA).

In some aspects, the surface cue used in a scaffold of the disclosure comprises a major histocompatibility complex (MHC) molecule which binds to CD3. Representative examples include, but are not limited to, MHC type I, which binds to TCR and CD8, and MHC type II, which binds to TCR and CD4. In some aspects, MHC molecules include HLA-A, HLA-B, HLA-C, DP, DQ, and DR, or a combination thereof. In some aspects, the surface cues comprise two or more MHC molecules attached to a linker. In some aspects, the MHC molecule is monovalent. In some aspects, the MHC molecule is bivalent.

In some aspects, the MHC molecules are loaded with a specific peptide (e.g., a peptide derived from a viral antigen, a bacterial antigen, allergen antigen, or tumor-associated antigen).

In some aspects, the surface cue comprises a fusion protein. In some aspects, the fusion protein has T cell stimulatory properties. T cell stimulatory properties can be constructed by using a linker which allows for delivery of a second signal to the T cell in addition to the signal delivered via the TCR. This can be accomplished by using a linker that has binding affinity for a cell surface structure on another cell, that cell being capable of delivering a second signal to the T cell. Thus, the linker serves to bridge the T cell and the other cell. By bringing the other cell into close proximity to the T cell, the other cell can deliver a second signal to the T cell.

In some aspects, the surface cue of the disclosure comprises one or more co-stimulatory molecules. As used herein “co-stimulatory molecule” refers to a polypeptide that binds to and provides a secondary or co-stimulatory signal to a cell, such as an immune cell (e.g., a T cell). Some co-stimulatory molecules include immune cell surface receptor/ligands, which engage between T cells and antigen presenting cells and generate a stimulatory signal in T cells, which combines with the stimulatory signal (i.e., “co-stimulation”) in T cells that results from T cell receptor (“TCR”) recognition of antigen on antigen presenting cells. As used herein, a soluble form of a co-stimulatory molecule “derived from an APC” refers to a co-stimulatory molecule normally expressed by B cells, macrophages, monocytes, dendritic cells and other APCs. See, Huppa et al., Nature Reviews Immunology. 3, 973-983 (2003). A “co-stimulator of T cell activation” refers to the ability of a co-stimulatory ligand to bind and to activate T cells which have been activated via any of the aforementioned mechanisms or pathways, e.g., via CD3-dependent or CD3-independent T-cell activation. Co-stimulatory activation can be measured for T cells by the production of cytokines and by proliferation assays that are well known (e.g., CFSE staining).

Such co-stimulatory molecules can mediate direct, indirect, or semi-direct stimulation of a target population of cells. In some aspects, the co-stimulatory molecules mediate activation of T-cells in the presence of one or more surface cues.

In some aspects, the co-stimulatory molecule comprises molecules that specifically bind to a co-stimulatory receptor (e.g., recombinant ligands, purified natural ligands, or derivatives thereof). In some aspects, the co-stimulatory molecule comprises an antibody or antigen-binding portion thereof, which binds specifically to one or more co-stimulatory antigens. Representative examples of co-stimulatory molecules include, but are not limited to, molecules that specifically bind to CD28, 4. IBB (CD137), OX40 (CD134), CD27 (TNFRSF7), GITR (CD357), CD30 (TNFRSF8), HVEM (CD270), LT R (TNFRSF3), DR3 (TNFRSF25), ICOS (CD278), CD226 (DNAM1), CRTAM (CD355),TIM1 (HAVCR1, KIM1), CD2 (LFA2, 0X34), SLAM (CD150, SLAMF1), 2B4 (CD244, SLAMF4), Lyl08 (NTBA, CD352, SLAMF6), CD84 (SLAMF5), Ly9 (CD229, SLAMF3), CD279 (PD-1) and/or CRACC (CD319, BLAME).

In this context, CD28 is the prototypic T cell co-stimulatory receptor and binds to molecules of the B7 family expressed on APCs such as dendritic cells and activated B cells. The ligands for CD28 include CD80 (B7-1) and CD86 (B7-2), which are immunoglobulin superfamily monomeric transmembrane glycoproteins.

In some aspects, the co-stimulatory molecule comprises an anti-CD28 antibody or antigen-binding portion thereof. In some aspects, the co-stimulatory molecule comprises an anti-ICOS (CD278) antibody or antigen-binding portion thereof. In some aspects, the co-stimulatory molecule comprises an anti-CD152 (CTLA4) antibody or antigen-binding portion thereof. In some aspects, the co-stimulatory molecule comprises an anti-CD81 antibody or antigen-binding portion thereof. In some aspects, the co-stimulatory molecule comprises an anti-CD137 antibody or antigen-binding portion thereof. In some aspects, the co-stimulatory molecule comprises an anti-OX40 (CD134) antibody or antigen-binding portion thereof. In some aspects, the co-stimulatory molecule comprises an anti-CD27 (TNFRSF7) antibody or antigen-binding portion thereof. In some aspects, the co-stimulatory molecule comprises an anti-GITR (CD357) antibody or antigen-binding portion thereof. In some aspects, the co-stimulatory molecule comprises an anti-CD30 (TNFRSF8) antibody or antigen-binding portion thereof. In some aspects, the co-stimulatory molecule comprises an anti-HVEM (CD270) antibody or antigen-binding portion thereof. In some aspects, the co-stimulatory molecule comprises an anti-LTpR (TNFRSF3) antibody or antigen-binding portion thereof. In some aspects, the co-stimulatory molecule comprises an anti-DR3 (TNFRSF25) antibody or antigen-binding portion thereof. In some aspects, the co-stimulatory molecule comprises an anti-CD226 (DNAM1) antibody or antigen-binding portion thereof. In some aspects, the co-stimulatory molecule comprises an anti-CRTAM (CD355) antibody or antigen-binding portion thereof. In some aspects, the co-stimulatory molecule comprises an anti-TIM1 (HAVCR1, KIM1) antibody or antigen-binding portion thereof. In some aspects, the co-stimulatory molecule comprises an anti-SLAM (CD 150, SLAMF1) antibody or antigen-binding portion thereof. In some aspects, the co-stimulatory molecule comprises an anti-2B4 (CD244, SLAMF4) antibody or antigen-binding portion thereof. In some aspects, the co-stimulatory molecule comprises an anti-Lyl08 (NTBA, CD352, SLAMF6). In some aspects, the co-stimulatory molecule comprises an anti-CD84 (SLAMF5) antibody or antigen-binding portion thereof. In some aspects, the co-stimulatory molecule comprises an anti-CD229 (Ly9, SLAMF3) antibody or antigen-binding portion thereof. In some aspects, the co-stimulatory molecule comprises an anti-PD-1 (CD279). In some aspects, the co-stimulatory molecule comprises an anti-CRACC (CD319, BLAME) antibody or antigen-binding portion thereof. Representative examples of co-stimulatory molecules include, but are not limited to, those referenced in e.g., U.S. Pat. No. 8,785,604; Int'l Publication No. WO 2010/078526; Maecker et al., BMC Immunol., 4:1, (2003); Ramakrishna et al., Journal for ImmunoTherapy of Cancer, 3:37, (2015); Cheung et al, J. Immunol, 185:1949, (2010); Hobo et al, J. Immunol. 189:39, (2012); Reddy et al, J. Virol, 86 (19) 10606-10620, (2012); Wolf et al., Transplantation, 27; 94(6):569-74, (2012); Flaig et al., J. Immunol. 172:6524-6527, (2004); and Stark et al., J. Immunol. Methods 296: 149-158, (2005), each of which is incorporated by reference herein in its entirety. In some aspects, the co-stimulatory molecule comprises a recombinant or purified natural ligand or derivative thereof.

In some aspects, the scaffolds comprise a pair of surface cues. In some aspects, a pair of surface cues provide a primary stimulatory signal and co-stimulatory signal to a target cell, such as a T cell. Representative examples of such pairs include, but are not limited to, antibodies capable of binding to CD3/CD28, CD3/ICOS, CD3/CD27, and CD3/CD137, or a combination thereof.

In some aspects, the scaffolds comprise a binding pair comprising an antibody binding to CD3 and at least one co-stimulatory molecule. In some aspects, the at least one co-stimulatory molecule comprises an anti-CD28 antibody. In some aspects, the at least one co-stimulatory molecule comprises an anti-CD28 antibody and a second co-stimulatory molecule. In some aspects, the second costimulatory molecule comprises an antibody that specifically binds ICOS, CD27, or CD137. In some aspects, the scaffold comprises a combination of functional molecules selected from the following combinations: (a) antibodies that specifically bind CD3, CD28, and ICOS, (b) antibodies that specifically bind to CD3, CD28, and CD27, (c) antibodies that specifically bind to CD3, CD28, and CD137, (d) antibodies that specifically bind to CD3, CD28, ICOS and CD27.

In some aspects, the scaffolds comprise a binding pair comprising at least two monospecific antibodies, wherein a first antibody binds to a first member of the pair, e.g., CD3, and a second antibody binds to a second member of the pair, e.g., CD28. In some aspects, the binding pair comprises a bispecific antibody comprising an antigen-binding domain that specifically binds CD3 and an antigen-binding domain that specifically binds CD28.

Alternately, in some aspects, the binding pair comprises at least two monospecific antibodies, wherein a first antibody binds to CD3 and a second antibody binds to ICOS. In some aspects, the binding pair comprises an antibody or antigen-binding portion thereof the specifically binds to ICOS. In some aspects, the antibody is an antagonistic antibody or antigen-binding portion that neutralizes ICOS.

In some aspects, the binding pair comprises at least two monospecific antibodies, wherein a first antibody binds to CD3 and a second antibody binds to CD27. In some aspects, both antibodies are stimulatory antibodies. In some aspects, both antibodies are agonist antibodies. In some aspects, the binding scaffold comprises a bispecific antibody comprising an agonist anti-CD3 binding domain and an agonist CD27 binding domain.

In some aspects, the binding pair comprises at least two monospecific antibodies, wherein a first antibody binds to CD3 and a second antibody binds to CD137. In some aspects, both antibodies are stimulatory antibodies. In some aspects, both antibodies are agonist antibodies. In some aspects, the binding scaffold comprises a bispecific antibody comprising an agonist anti-CD3 binding domain and an agonist anti-CD137 binding domain.

In some aspects, the scaffold comprises a plurality of surface cues. In one aspect, the scaffold comprises multiple antibodies where each antibody preferentially binds to a different receptor on the surface of a target cell.

The amount of different surface cue molecules present on the scaffolds, such as surface cue 1/surface cue 2, for example, can be understood as functional molecule density, calculated as either the theoretical number of molecules per surface area or scaffold or calculated based on the mol percent of coating lipid used for functional molecule presentation or stoichiometry of said functional molecules. The surface cue density can be determined by the percentage of the lipids in the layer comprising lipids being used for function molecule affinity pairing, wherein the surface cues are affixed to the layer comprising lipids. The ratio or stoichiometry of said functional molecules can be expressed as the relative proportion of the various functional molecules being affixed. The density of functional molecule presentation can also be determined by the dry weight ratio of the MSR to the dry weight of the combined surface cues.

The term “affinity pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, biotin/biotin binding agent, Ni2+ or Cu2+ chelator (e.g., NTA or other chelator/metal pair)/HisTag (6× histidine or other polyhistidine tag) and virus-receptor interactions. Various other specific binding pairs are contemplated for use in practicing the methods of this disclosure.

As used herein, “biotin-binding agent” encompasses avidin, streptavidin and other avidin analogs such as streptavidin or avidin conjugates, highly purified and fractionated species of avidin or streptavidin, and non or partial amino acid variants, recombinant or chemically synthesized avidin analogs with amino acid or chemical substitutions, which still accommodate biotin binding.

In some aspects, each biotin-binding agent molecule binds at least two biotin moieties. In some aspects, each biotin-binding agent molecule binds at least four biotin moieties. As used herein, “biotin” encompasses biotin in addition to biocytin and other biotin analogs such as biotin amido caproate N-hydroxysuccinimide ester, biotin 4-amidobenzoic acid, biotinamide caproyl hydrazide and other biotin derivatives and conjugates. Other derivatives include biotin-dextran, biotin-disulfide-N-hydroxysuccinimide ester, biotin-6 amido quinoline, biotin hydrazide, d-biotin-N hydroxysuccinimide ester, biotin maleimide, d-biotin p-nitrophenyl ester, biotinylated nucleotides and biotinylated amino acids such as Np-biotinyl-1-lysine.

The ligands that can be functionalized via affinity pairing include, but are not limited to, receptors, monoclonal or polyclonal antibodies, viruses, chemotherapeutic agents, receptor agonists and antagonists, antibody fragments, lectin, albumin, peptides, proteins, hormones, amino sugars, lipids, fatty acids, nucleic acids, and cells prepared or isolated from natural or synthetic sources. Any site-specific ligand for any molecular epitope or receptor to be detected through the practice of the disclosure can be utilized. In some aspects, the ligand is a membrane-anchored protein.

The functional molecules, as noted hereinabove, can be any protein or peptide. In some aspects, the proteins are involved in ligand-receptor interactions. For example, an important event of T cell activation is a result of membrane-membrane contact between T cells and APCs, wherein a variety of ligand-receptor interactions take place between the two opposing membranes, including, MHC-peptide and TCR, LFA-1 and ICAM-1, CD2 and CD48, as well as B7 or CTLA-4 and CD28.

Incorporation of predefined amounts of a biotinylated phospholipid into liposome formulations enables the precise surface attachment of biotinylated surface cues via streptavidin-biotin interactions, mimicking the cell surface presentation of cues by natural APCs to T cells.

In some aspects, the density of surface cues or the combinations of surface cues is determined by percentage of affinity paired (e.g., biotinylated) lipid used in the scaffold. In some aspects, the percentage of biotinylated lipid is between about 0.01% to about 1.1%. In some aspects, the percentage of biotinylated lipid is between about 0.1% to about 0.9%. In some aspects, the percentage of biotinylated lipid is between about 0.1% to about 2.5%. In some aspects, the percentage of biotinylated lipid is about 0.01%. In some aspects, the percentage of biotinylated lipid is about 0.05%. In some aspects, the percentage of biotinylated lipid is about 0.1%. In some aspects, the percentage of biotinylated lipid is about 0.2%. In some aspects, the percentage of biotinylated lipid is about 0.25%. In some aspects, the percentage of biotinylated lipid is about 0.3%. In some aspects, the percentage of biotinylated lipid is about 0.4%. In some aspects, the percentage of biotinylated lipid is about 0.5%. In some aspects, the percentage of biotinylated lipid is about 0.6%. In some aspects, the percentage of biotinylated lipid is about 0.7%. In some aspects, the percentage of biotinylated lipid is about 0.8%. In some aspects, the percentage of biotinylated lipid is about 0.9%. In some aspects, the percentage of biotinylated lipid is about 1.0%. In some aspects, the percentage of biotinylated lipid is about 1.1%. In some aspects, the percentage of biotinylated lipid is about 1.5%. In some aspects, the percentage of biotinylated lipid is about 2.0%. In some aspects, the percentage of biotinylated lipid is about 2.5%.

In some aspects, the density of surface cues or the combination of surface cues is determined by the mass of each affinity paired surface cue added during scaffold loading, provided there is an excess of affinity paired lipid in the scaffold, e.g., when using a metal-chelating lipid and his-tagged surface cue.

In some aspects, the dry weight ratio of the MSR to the surface cues (stoichiometry) is from about 1:1 to about 100:1. In some aspects, the dry weight ratio of the MSR to the surface cues (stoichiometry) is from about 10:1 to about 50:1. In some aspects, the dry weight ratio of the MSR to the surface cues (stoichiometry) is from about 20:1 to about 50:1. In some aspects, the dry weight ratio of the MSR to the surface cue of the scaffolds is from about 10,000:1 to about 1:1. In some aspects, the dry weight ratio of the MSR to the surface cues of the scaffolds is from about 5,000:1 to about 1:1, from about 1,000:1 to about 1:1, from about 500:1 to about 1:1, or from about 100:1 to about 1:1. In some aspects, the dry weight ratio of the MSR to the surface cues of the scaffolds is about 10,000:1, about 5,000:1, about 2,500:1, about 1,000:1, about 750:1, about 500:1, about 250:1, about 100:1, about 75:1, about 50:1, about 40:1, about 30:1, about 25:1, about 20:1, about 10:1, or about 1:1.

II.B.6. Soluble Cues

In some aspects, the scaffolds comprise a plurality of soluble cues. As used herein “soluble cues” refers to cell-signaling molecules in contact with the scaffold structure. In some aspects, the soluble cues are in contact with, or coupled to, the layer comprising MSR of the scaffold structure. In some aspects, the scaffolds of the instant disclosure contain a plurality of soluble cues selected from the group consisting of IL-1, IL-2, IL-4, IL-5, IL-7, IL-10, IL-12, IL-15, IL-17, IL-21, Wnt proteins, and transforming growth factor beta (TGF-β), or an agonist thereof, a mimetic thereof, a variant thereof, a functional fragment thereof, or a combination thereof.

Representative soluble cues, include, but are not limited to, the following NCBI accession numbers of human and/or mouse homologs thereof: IL-1, NP_000566.3 (human); IL-1α, NP_034684.2 (mouse); IL-1, NP_000567.1 (human); IL-1β, NP_032387.1 (mouse); IL-2, NP_000577.2 (human) and NP_032392.1 (mouse); IL-4, NP_000580.1, NP_758858.1 (human) and NP_067258.1 (mouse); IL-5, NP_000870.1 (human) and NP_034688.1 (mouse); IL-7, NP_000871.1, NP_001186815.1, NP_001186816.1, NP_001186817.1 (human) and NP_032397.1 (mouse); IL-10, NP_000563 (human) and NP_034678.1 (mouse); IL-12A, NP_000873.2 (human) and NP_001152896.1, NP_032377.1 (mouse); IL-12B, NP_002178.2 (human) and NP_001290173.1 (mouse); IL-15, NP_000576.1, NP_751915.1 (human) and NP_001241676.1, NP_032383.1 (mouse); IL-17(a), NP_002181.1, NP_034682.1 (human); NP_002181.1; NP_034682.1 (mouse); TGF-beta 1, NP_000651.3 (human) and NP_035707.1 (mouse); TGF-beta 2, NP_001129071.1, NP_003229.1 (human) and NP_033393.2 (mouse); and TGF-beta, NP_003230.1 (human). Non-limiting examples of fragments and variants of the aforementioned soluble cues are presented, for example, in the database UNIPROT.

In some aspects, the soluble cue comprises interleukin-2 (IL-2) or an agonist thereof, a mimetic thereof, a variant thereof, a functional fragment thereof, or a combination thereof with one or more additional soluble cues listed above. Non-limiting examples of IL-2 agonists, mimetics thereof, variants thereof, and functional fragments thereof include those provided in U.S. Pat. Nos. 5,496,924; 6,955,807; Margolin et al, Clin Cancer Res. 1; 13(11):33 12-9 (2007); Eckenberg et al, J Immunol 165:4312-4318 (2000); Levin et al, Nature 484, 529-533, (2012); and Zurawski et al., EMBO Journal, 9(12): 3899-3905 (1990), each of which is incorporated herein by reference in its entirety.

In some aspects, the scaffolds comprise a plurality of soluble cues. In some aspects, the scaffold comprises a first soluble cue comprising IL-2 and a second soluble cue comprising IL-7, IL-21, IL-15, or IL-15 superagonist. IL-15 superagonist (IL-15 SA) is a combination of IL-15 with soluble IL-15 receptor-a, which possesses greater biological activity than IL-15 alone. In some aspects, the scaffold comprises a first soluble cue comprising IL-2, a second soluble cue comprising IL-7, and a third soluble cue comprising IL-15. In some aspects, the scaffold comprises a first soluble cue comprising IL-2 and a second and third soluble cue comprising IL-7, IL-21, IL-15, or IL-15 superagonist.

In some aspects, the total soluble cue input to MSR mass ratio (μg total soluble cue input to μg MSR) is about 0.001 to about 0.005. In some aspects, the total soluble cue input to MSR mass ratio is about 0.001. In some aspects, the total soluble cue input to mass ratio is about 0.002. In some aspects, the total soluble cue input to MSR mass ratio is about 0.003. In some aspects, the total soluble cue input to MSR mass ratio is about 0.004. In some aspects, the total soluble cue input to MSR mass ratio is about 0.005. In some aspects, wherein a scaffold comprises more than one soluble cue, the cues are present in equal amounts. In some aspects, the scaffold comprises more than one soluble cue, wherein the cues are present in unequal amounts.

II.B.7. Further Aspects of Scaffolds

In some aspects the scaffolds comprise a plurality of surface cues and soluble cues. A typical scaffold can comprise at least 2, at least 3, at least 4, at least 5, at least 6, at least 7, at least 8, at least 9, at least 10, at least 11, or more of each of the aforementioned functional molecules.

In some aspects, the functional molecules are recombinant. In some aspects, the functional molecules are humanized derivatives of mammalian counterparts. Exemplary mammalian species from which the functional molecules are derived include, but are not limited to, mouse, rat, hamster, guinea pig, ferret, cat, dog, monkey, or primate. In some aspects, the functional molecules are human or humanized version of the aforementioned functional molecules.

The functional molecules can be modified to increase protein stability in vivo. Alternatively, the functional molecules can be engineered to be more or less immunogenic. For instance, insofar as the structures of the various functional molecules are known, the sequences can be modified at one or more of amino acid residues, e.g., glycosylation sites, to generate immunogenic variants.

Any functional molecule (e.g., any antigen, antibody, protein, enzyme, fragment thereof, recombinant or purified natural ligands or derivatives thereof, or any combination thereof) can be directly or indirectly immobilized onto the layer comprising MSR and/or the layer comprising lipids using routine techniques. In some aspects, the functional molecules are provided in an organelle (e.g., golgi membrane or plasma membrane), a cell, a cell cluster, a tissue, a microorganism, an animal, a plant, or an extract thereof, which in turn is immobilized onto the layer comprising MSR or the layer comprising lipids. In some aspects, the functional molecule is synthesized by genetic engineering or chemical reactions at the desired situs, e.g., outer face of the layer comprising lipids.

Each of the aforementioned functional molecules, e.g., surface cues and soluble cues can, independently from one another, be loaded, adsorbed or integrated into/onto the layer comprising MSR or the layer comprising lipids. Therefore, in some aspects, the surface cues are loaded, adsorbed or integrated into/onto the layer of the scaffold comprising MSR. In one aspect, the surface cues are loaded, adsorbed or integrated into/onto the layer comprising lipids. In some aspects, the surface cues are loaded, adsorbed or integrated into/onto both the layer comprising MSR as well as the layer comprising lipids. In some aspects, the surface cue comprises a co-stimulatory molecule loaded, adsorbed or integrated into/onto the layer comprising MSR. In some aspects, the co-stimulatory molecule is loaded, adsorbed or integrated into/onto the layer comprising lipids. In some aspects, the surface cue comprises a co-stimulatory molecule, which is loaded, adsorbed or integrated into/onto both the layer comprising MSR as well as the layer comprising lipids. In some aspects, the soluble cues are loaded, adsorbed or integrated into/onto the layer comprising MSR. In some aspects, the soluble cues are loaded, adsorbed or integrated into/onto the layer comprising lipids. In some aspects, the soluble cues are loaded, adsorbed or integrated into/onto both the layer comprising MSR as well as the layer comprising lipids.

In general, the functional molecules and the layer comprising MSR and/or the layer comprising lipids, can be linked together through the use of reactive groups, which are typically transformed by the linking process into a new organic functional group or unreactive species. The reactive functional group(s) can be located in any of the aforementioned components. Reactive groups and classes of reactions useful in practicing the present disclosure are generally those that are well known in the art of bioconjugate chemistry. Currently favored classes of reactions available with reactive chelates are those that proceed under relatively mild conditions. These include, but are not limited to, nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). In some aspects, chemical coupling comprises click chemistry, discussed in, for example, clickchemistrytools.com. In some aspects, chemical coupling comprises a click chemistry reagent (e.g., DBCO or azide). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al, Modification of Proteins; vol. 198, American Chemical Society, Washington, D.C., 1982.

In some aspects, the scaffolds comprise one or more additional molecules. In some aspects, the one or more additional molecules are naturally-occurring, synthetically produced, or recombinant compounds. In some aspects, the one or more additional molecules comprise peptides, polypeptides, nucleic acids, small molecules, haptens, carbohydrates, or agents, including fragments thereof or combinations thereof.

In some aspects, an anchor is used to connect a functional molecule to a pore wall. However, the anchor is not an essential component. In some aspects, each pore of the mesoporous silica accommodates at least one functional molecule. The pore size depends on the size of the functional molecule to be immobilized. In some aspects, the functional molecule is immobilized in a pore. In some aspects, the functional molecule is loaded or adsorbed on an inner surface of the pore by electrostatic bonding. In some aspects, the functional molecule is loaded or adsorbed on an inner surface of the pore by a noncovalent bond.

In some aspects, the anchor reduces a large structural change of the functional molecule to hold it stably. In some aspects, the anchor comprises substantially the same component as the mesoporous material. In some aspects, the anchor comprises one or more functional groups to permit binding to a desired functional molecule: a hydroxyl group, an amide group, an amino group, a pyridine group, a urea group, a urethane group, a carboxyl group, a phenol group, an azo group, a hydroxyl group, a maleimide group, a silane derivative, an aminoalkylene group, or a combination thereof.

In some aspects, a scaffold comprises an antigen. In some aspects, the antigen comprises a polypeptide. In some aspects, the antigen is purified. In some aspects, the antigen is a self-antigen. In some aspects, the antigen is a non-self antigen. Self-antigens are specifically associated with a human disease or a disorder including, but not limited to, autoimmune disorders and cancer. Non-self antigens are specifically associated with pathogens including, but not limited to, a virus, a bacteria, a protozoan, a parasite, or a fungus. In some aspects, the antigens are loaded onto MHC molecules, e.g., HLA-A, HLA-B, HLA-C, DP, DQ, and DR, which are then incorporated into/onto the scaffolds.

In some aspects, the antigen is formulated to interact with the immune cell via direct binding or indirect binding. Types of direct binding include, for example, engagement or coupling of the antigen with the cognate receptor, e.g., T-cell receptor. Indirect binding can occur through the intermediacy of one or more secondary agents or cell-types. For example, the antigen can first bind to a B-cell or an antigen-presenting cell (APC), get processed (e.g., degraded) and presented on cell-surface major-histocompatibility complexes (MHC), to which the target cell population, e.g., T-cell, binds. Alternately, the antigen can recruit other intermediary cells that secrete various cytokines, growth factors, chemokines, etc., which in turn attract the target immune cell population. In some aspects, the antigen is CD19, CD22, or a fragment thereof.

In some aspects, the scaffold comprises a membrane-associated protein, which is anchored directly or indirectly to the layer comprising lipids. In some aspects, the membrane-associated protein comprises a selective or non-selective membrane transport protein, ion channel, pore forming protein, membrane-resident receptors, or any combination thereof.

In some aspects, the scaffold comprises a growth factor, a cytokine (e.g., IL-2, IL-7, IL-15, and/or IL-21), a chemokine, an interleukin, an adhesion signaling molecule, an integrin signaling molecule, a fragment thereof, or any combination thereof. In some aspects, these molecules can be used as soluble cues and/or surface cues and can be loaded to either the layer comprising the MSR or the layer comprising the lipids.

In some aspects, the scaffold comprises adhesion molecules. In some aspects, the adhesion molecules further serve as signaling agents. Representative examples of adhesion signaling molecules include, but are not limited to, fibronectin, laminin, collagen, thrombospondin 1, vitronectin, elastin, tenascin, aggrecan, agrin, bone sialoprotein, cartilage matrix protein, fibrinogen, fibrin, fibulin, mucins, entactin, osteopontin, plasminogen, restrictin, serglycin, SPARC/osteonectin, versican, von Willebrand Factor, polysaccharide heparin sulfate, connexins, collagen, RGD (Arg-Gly-Asp) and YIGSR (Tyr-Ile-Gly-Ser-Arg) peptides and cyclic peptides, glycosaminoglycans (GAGs), hyaluronic acid (HA), chondroitin-6-sulfate, integrin ligands, selectins, cadherins, and members of the immunoglobulin superfamily.

In some aspects, the functional molecules are conjugated to membrane-associated proteins, which associate with and/or insert into the layer comprising lipids, e.g. gramicidin; a-helix bundles, e.g. bacteriorhodopsin or K+ channels; β-barrels, e.g., a-hemolysin, leucocidin or E. coli porins; or combinations thereof.

In some aspects, the scaffold further comprises one or more recruiting agents. In some aspects, the recruiting agent comprises an agent selected from the group consisting of a T-cell recruiting agent, a B-cell recruiting agent, a dendritic cell recruiting agent, and a macrophage recruiting agent. Examples of such recruiting agents include, but are not limited to chemokines, chemokine ligands, or fragments, variants, homologs, and combinations thereof. Preferential recruitment is characterized by an accumulation of at least 10%, at least 20%, at least 30%, at least 50%, at least 75%, at least 100%, at least 2-fold, at least 5-fold, at least 8-fold, at least 10-fold, or greater increase in one or more of a particular type of immune cells compared to other types of immune cells.

Depending on need, the scaffolds can be specifically formulated to comprise a subset of recruitment agents and adhesion molecules so as to manipulate a particular subset of immune cells, e.g., pan-T cells or a particular sub-population of T-cells. In some aspects, the scaffolds are formulated/fabricated using agents that specifically bind to cell-surface markers that are expressed in the target cells. For example, in the context of T-cells, the scaffolds can be adapted for the preferential recruitment of helper T-cells (CD4⁺ T cells), cytotoxic T-cells (CD8⁺ T cells), memory T-cells (CD45RO⁺ T cells), suppressor T-cells (Ts which cells), regulatory T-cells (Tregs; further characterized as FOXP3+ Treg cells and FOXP3− Treg), natural killer T-cells (NK cells; differentially express CDld+), mucosal associated invariant (MAITs; differentially express MR1), gamma delta T cells, (γδ T cells; comprise TCRs containing one γ-chain and one δ-chain). Such agents which bind to cell-surface markers can include, for example, haptens, peptides, ligands, antibodies, or the like, or any combination thereof. Other routine techniques for enriching the isolates with one or more cell subtype can be used in situ or ex situ.

II.B.8. Methods of Making

The scaffolds of the disclosure can be generated in a variety of ways and used for various applications, including, but not limited to, modulating the type and abundance of functional molecules or additional agents in accordance with a scaffold, for use in the manipulation of target effector cells, e.g., T-cells, isolation of a specific population of effector cells, e.g., a sub-population of CD8+ T-cells, therapy of diseases, and the production of compositions and kits. Examples of methods of making and using such scaffolds is described in PCT Publication No. WO 2018/013797 A1 and Chung et al. (Nature Biotechnology 36(2): 160-169 (2018)), the entire contents of which are incorporated by reference herein.

For isolation and/or expansion of a desired population of cells, the concentration of cells and scaffold surface can be varied. In some aspects, the volume in which the scaffolds and cells are mixed is decreased (i.e., increasing the concentration of cells), to ensure maximum contact of cells and scaffolds. In some aspects, a concentration of 2 billion cells/ml is used. In some aspects, a concentration of 1 billion cells/ml is used. In a further aspect, greater than 100 million cells/ml is used. In a further aspect, a concentration of cells of 10 million, 15 million, 20 million, 25 million, 30 million, 35 million, 40 million, 45 million, or 50 million cells/ml is used. In yet another aspect, a concentration of cells from 75 million, 80 million, 85 million, 90 million, 95 million, or 100 million cells/ml is used. In further aspects, a concentration of 125 million or 150 million cells/ml is used. Using high concentrations can result in increased cell yield, cell activation, and cell expansion. Further, use of high cell concentrations can allow more efficient capture of cells that can weakly express target antigens of interest, such as CD28− negative T cells, or from samples where there are many tumor cells present (i.e., leukemic blood, tumor tissue, etc.). Such populations of cells can have a therapeutic value and would be desirable to obtain. For example, using high concentration of cells allows more efficient selection of CD8+ T cells that normally have weaker CD28 expression.

In other aspects, it is desirable to use lower concentrations of cells. This can be achieved by lowering the scaffold:cell ratio, such that interactions between the scaffolds and cells are minimized. This method selects for cells that express high amounts of desired antigens to be bound to the scaffolds. For example, CD4+ T cells express higher levels of CD28 and are more efficiently captured than CD8+ T cells in dilute concentrations. In some aspects, the concentration of cells used is 5×10⁶/ml. In other aspects, the concentration used can be from about 1×10⁴/ml to 1×10⁹/ml, and any integer value in between, e.g., 1×10⁵/ml to 1×10⁸/ml, 1×10⁶/ml to 1×10⁷/ml, 1×10⁷/ml to 1×10⁹/ml.

II.C. Cells

Some aspects of the present disclosure are directed to methods of culturing immune cells, e.g., T cells and/or NK cells, comprising contacting the immune cells with programmable cell-signaling scaffolds (PCS) in a medium comprising potassium ion at a concentration of higher than 5 mM, as disclosed herein. The immune cells, e.g., T cells and/or NK cells, that are placed in the medium can be cells that are collected and/or isolated from a subject in need of a therapy. In some aspects, the immune cells, e.g., T cells and/or NK cells, that are placed in the medium have been engineered prior to the culturing. In some aspects, the immune cells, e.g., T cells and/or NK cells, that are placed in the medium have been expanded. The immune cells, e.g., T cells and/or NK cells, that are placed in the medium can be referred to as starting (initial, i.e., patient sample, apheresis sample, buffy coat) cells. The immune cells, e.g., T cells and/or NK cells, that result from culturing them in the metabolic reprogramming media disclosed herein can be referred to as resulting (cultured or expanded) cells.

The methods disclosed herein provide culture conditions that promote a less-differentiated phenotype for cultured immune cells, e.g., T cells and/or NK cells. In some aspects, the starting immune cells, e.g., T cells and/or NK cells, are isolated from a human subject. In some aspects, the starting immune cells, e.g., T cells and/or NK cells, are isolated from a human subject for allogeneic cell therapy. In some aspects, the starting immune cells, e.g., T cells and/or NK cells, are isolated from a human subject for autologous cell therapy. In some aspects, the immune cells are T cells. In some aspects, the immune cells are NK cells. In some aspects, the immune cells are TILs. In some aspects, the immune cells are Tregs. In some aspects, the immune cells, e.g., T cells and/or NK cells, are isolated from a human subject. In some aspects, the immune cells are tumor-infiltrating T cells or tumor-infiltrating NK cells. In certain aspects, the immune cells, e.g., T cells and/or NK cells, are engineered. In some aspects, the immune cells, e.g., T cells and/or NK cells, are engineered to comprise a chimeric antigen receptor (CAR). In some aspects, the immune cells, e.g., T cells and/or NK cells, are engineered to comprise an engineered T cell receptor (TCR).

In some aspects, the cells, e.g., T cells, NK cells, and/or TILs, are engineered before culturing according to the methods disclosed herein. In some aspects, the cells, e.g., T cells, NK cells, and/or TILs, are engineered after culturing according to the methods disclosed herein. In some aspects, the cells, e.g., T cells, NK cells, and/or TILs, are cultured according to the methods disclosed herein, e.g., in a hypotonic or isotonic medium comprising at least 5 mM potassium ion, prior to, during, and after cell engineering. In some aspects, the cells, e.g., T cells, NK cells, and/or TILs, are engineered to express a chimeric antigen receptor (CAR). In some aspects, the cells, e.g., T cells, NK cells, and/or TILs, are engineered to express an engineered T cell receptor (TCR). In certain aspects, culturing the cells, e.g., T cells, NK cells, and/or TILs, under the conditions disclosed herein, e.g., in a hypotonic or isotonic medium comprising at least about 5 mM potassium ion, results in higher transduction efficiency. In some aspects, transduction efficiency is at least about 2-fold greater in cells, e.g., T cells, NK cells, and/or TILs, cultured in hypotonic or isotonic medium comprising at least about 60 mM potassium ion, according to the methods disclosed herein, as compared to cells, e.g., T cells, NK cells, and/or TILs, cultured in medium comprising 4 mM potassium ion or less. In some aspects, transduction efficiency is at least about 2.5-fold greater in cells, e.g., T cells, NK cells, and/or TILs, cultured in hypotonic or isotonic medium comprising at least about 65 mM potassium ion, according to the methods disclosed herein, as compared to cells, e.g., T cells, NK cells, and/or TILs, cultured in medium comprising 4 mM potassium ion or less.

In some aspects, the cell comprises a construct expressing an antigen receptor and/or another additional polypeptide. In some aspects, the antigen receptor comprises an antibody, an engineered antibody such as scFv, a CAR, an engineered TCR, a TCR mimic (e.g., an antibody-T cell receptor (abTCR) or a chimeric antibody-T cell receptor (caTCR)), or a chimeric signaling receptor (CSR). By way of example, a TCR can comprise an engineered TCR in which the antigen-binding domain of a TCR (e.g., an alpha/beta TCR or a gamma/delta TCR) has been replaced by that of an antibody (with or without the antibody's constant domains); the engineered TCR then becomes specific for the antibody's antigen while retaining the TCR's signaling functions. A chimeric signaling receptor can comprise (1) an extracellular binding domain (e.g., natural/modified receptor extracellular domain, natural/modified ligand extracellular domain, scFv, nanobody, Fab, DARPin, and affibody), (2) a transmembrane domain, and (3) an intracellular signaling domain (e.g., a domain that activates transcription factors, or recruits and/or activates JAK/STAT, kinases, phosphatases, and ubiquitin; SH3; SH2; and PDZ). See, e.g., EP340793B1, WO 2017/070608, WO 2018/200582, WO 2018/200583, WO 2018/200585, and Xu et al., Cell Discovery (2018) 4:62.

In some aspects, the construct expressing an antigen receptor and/or another additional polypeptide comprises a regulatory element, and wherein a vector comprises the exogenous polynucleotide. In some aspects, the vector is a polycistronic expression vector. In some aspects, the regulatory element comprises a promoter. In some aspects, the promoter comprises a dl587rev primer-binding site substituted (MND) promoter, EF1a promoter, ubiquitin promoter, or combinations thereof. In some aspects, the vector comprises a viral vector, a mammalian vector, or a bacterial vector. In some aspects, the vector comprises an adenoviral vector, a lentivirus, a Sendai virus vector, a baculoviral vector, an Epstein Barr viral vector, a papovaviral vector, a vaccinia viral vector, a herpes simplex viral vector, a hybrid vector, or an adeno associated virus (AAV) vector. In some aspects, the vector is a lentivirus.

In some aspects, the antigen receptor targets an antigen of interest (e.g., a tumor antigen or an antigen of a pathogen). The antigens can include, without limitation, AFP (alpha-fetoprotein), avP6 or another integrin, BCMA, B7-H3, B7-H6, Braf, CA9 (carbonic anhydrase 9), CCL-1 (C—C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens.

In certain aspects, the antigen receptor targets hTERT. In some aspects, the antigen receptor targets KRAS. In some aspects, the antigen receptor targets Braf. In some aspects, the antigen receptor targets TGFβRII. In some aspects, the antigen receptor targets MAGE A10/A4. In some aspects, the antigen receptor targets AFP. In some aspects, the antigen receptor targets PRAME. In some aspects, the antigen receptor targets MAGE A1. In some aspects, the antigen receptor targets WT-1. In some aspects, the antigen receptor targets NY-ESO. In some aspects, the antigen receptor targets PRAME. In some aspects, the antigen receptor targets NY-ESO. In some aspects, the antigen receptor targets CD19.

In some aspects, the antigen receptor targets BCMA. In some aspects, the antigen receptor targets CD147. In some aspects, the antigen receptor targets CD19. In some aspects, the antigen receptor targets CD19 and CD22. In some aspects, the antigen receptor targets CD19 and CD28. In some aspects, the antigen receptor targets CD20. In some aspects, the antigen receptor targets CD20 and CD19. In some aspects, the antigen receptor targets CD22. In some aspects, the antigen receptor targets CD30. In some aspects, the antigen receptor targets CEA. In some aspects, the antigen receptor targets DLL3. In some aspects, the antigen receptor targets EGFRvIII. In some aspects, the antigen receptor targets GD2. In some aspects, the antigen receptor targets HER2. In some aspects, the antigen receptor targets IL-1RAP. In some aspects, the antigen receptor targets mesothelin. In some aspects, the antigen receptor targets methothelin. In some aspects, the antigen receptor targets NKG2D. In some aspects, the antigen receptor targets PSMA. In some aspects, the antigen receptor targets TnMUC1.

In some aspects, the cells, e.g., T cells and/or NK cells, are engineered before culturing according to the methods disclosed herein. In some aspects, the cells, e.g., T cells and/or NK cells, are engineered after culturing according to the methods disclosed herein. In some aspects, the cells, e.g., T cells and/or NK cells, are cultured according to the methods disclosed herein, e.g., in a hypotonic or isotonic medium comprising at least 5 mM potassium ion, prior to, during, and after cell engineering. In some aspects, the cells, e.g., T cells and/or NK cells, are engineered to express a chimeric antigen receptor (CAR). In some aspects, the cells, e.g., T cells and/or NK cells, are engineered to express an engineered T cell receptor (TCR). In certain aspects, culturing the cells, e.g., T cells and/or NK cells, under the conditions disclosed herein, e.g., in a hypotonic or isotonic medium comprising at least about 5 mM potassium ion, results in higher transduction efficiency. In some aspects, transduction efficiency is at least about 2-fold greater in cells, e.g., T cells and/or NK cells, cultured in hypotonic or isotonic medium comprising at least about 60 mM potassium ion, according to the methods disclosed herein, as compared to cells, e.g., T cells and/or NK cells, cultured in medium comprising 4 mM potassium ion or less. In some aspects, transduction efficiency is at least about 2.5-fold greater in cells, e.g., T cells and/or NK cells, cultured in hypotonic or isotonic medium comprising at least about 65 mM potassium ion, according to the methods disclosed herein, as compared to cells, e.g., T cells and/or NK cells, cultured in medium comprising 4 mM potassium ion or less.

Primary immune cells, including primary T cells, can be obtained from a number of tissue sources, including peripheral blood mononuclear cells (PBMCs), bone marrow, lymph node tissue, cord blood, thymus tissue, tissue from a site of infection, ascites, pleural effusion, spleen tissue, and/or tumor tissue. Leukocytes, including PBMCs, can be isolated from other blood cells by well-known techniques, e.g., FICOLL™ separation and leukapheresis. Leukapheresis products typically contain lymphocytes (including T and B cells), monocytes, granulocytes, and other nucleated white blood cells. T cells are further isolated from other leukocytes, for example, by centrifugation through a PERCOLL™ gradient or by counterflow centrifugal elutriation. A specific subpopulation of T cells, such as CD3⁺, CD25⁺, CD28⁺, CD4⁺, CD8⁺, CD45RA⁺, GITR⁺, and CD45RO⁺ T cells, can be further isolated by positive or negative selection techniques (e.g., using fluorescence-based or magnetic-based cell sorting). For example, T cells can be isolated by incubation with any of a variety of commercially available antibody-conjugated beads, such as Dynabeads®, CELLection™, DETACHaBEAD™ (Thermo Fisher) or MACS® cell separation products (Miltenyi Biotec), for a time period sufficient for positive selection of the desired T cells or negative selection for removal of unwanted cells.

In some instances, autologous T cells are obtained from a cancer patient directly following cancer treatment. It has been observed that following certain cancer treatments, in particular those that impair the immune system, the quality of T cells collected shortly after treatment can have an improved ability to expand ex vivo and/or to engraft after being engineered ex vivo.

II.C.1. Chimeric Antigen Receptor (CAR)

In some aspects, the cell, e.g., human immune cell, e.g., T cell and/or NK cell, comprises a CAR. In some aspects, the cell that can be prepared to express a CAR (e.g., a CAR T cell) is, e.g., a CD8+ T cell or CD4+ T cell. In some aspects, a CAR-expressing cell disclosed herein is a CAR T cell, e.g., a mono CAR T cell, a genome-edited CAR T cell, a dual CAR T cell, or a tandem CAR T cell. Examples of such CAR T cells are provided in International Application No. PCT/US2019/044195.

In some aspects, the CAR is designed as a standard CAR, a split CAR, an off-switch CAR, an on-switch CAR, a first-generation CAR, a second-generation CAR, a third-generation CAR, or a fourth-generation CAR. In some aspects, the CAR comprises antigen-binding domain, a transmembrane domain, a costimulatory domain, an intracellular signaling domain, or combinations thereof.

In some aspects, the CAR specifically binds (i.e., target) one or more antigens expressed on a tumor cell, such as a malignant B cell, a malignant T cell, or a malignant plasma cell.

In some aspects, the CAR specifically binds to (i.e., targets) an antigen selected from the group consisting of AFP (alpha-fetoprotein), αvβ6 or another integrin, BCMA, Braf, B7-H3, B7-H6, CA9 (carbonic anhydrase 9), CCL-1 (C—C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof.

In some aspects, the CAR specifically binds ROR1. An exemplary anti-ROR1 CAR that can be expressed in an immune cell described herein is described in Hudecek, et al., Clin. Cancer Res. 19.12(2013):3153-64, which is incorporated herein by reference in its entirety. In some aspects, an immune cell modified to comprise an anti-ROR1 CAR is generated as described in Hudecek et al. (for example, as described in Hudecek et al. at page 3155, first full paragraph, incorporated herein by reference in its entirety). In some aspects, the spacer disclosed in Hudecek has been replaced by a different spacer (e.g., such as those described herein). In some aspect, an anti-ROR1 CAR useful for the present disclosure comprises an antibody or fragment thereof, which comprises the VH and/or VL sequences of the 2A2, R11, and R12 anti-ROR1 monoclonal antibodies described in Hudecek et al. at paragraph bridging pages 3154-55; Baskar et al. MAbs 4(2012):349-61; and Yang et al. PLoS ONE 6(2011): e21018, each of which is incorporated herein by reference in their entirety.

In some aspects, the CAR specifically binds GPC2.

In some aspects, the costimulatory domain comprises a costimulatory domain of an interleukin-2 receptor (IL-2R), interleukin-12 receptor (IL-12R), IL-7, IL-21, IL-23, IL-15, CD2, CD3, CD4, CD7, CD8, CD27, CD28, CD30, CD40, 4-1BB/CD137, ICOS, lymphocyte function-associated antigen-1 (LFA-1), LIGHT, NKG2C, OX40, DAP10, or any combination thereof. In some aspects, the costimulatory domain comprises a 4-1BB/CD137 costimulatory domain.

In some aspects, the transmembrane domain comprises a transmembrane domain of KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, CD19, or any combination thereof. In some aspects, the transmembrane domain comprises a CD28 transmembrane domain.

In some aspects, the intracellular signaling domain comprises an intracellular signaling domain derived from CD3 zeta, FcR gamma, common FcR gamma (FCER1G), Fc gamma RIIa, FcR beta (Fc Epsilon Rib), CD3 gamma, CD3 delta, CD3 epsilon, CD22, CD79a, CD79b, CD278 (“ICOS”), FcεRI, CD66d, CD32, DAP10, DAP12, or any combination thereof. In some aspects, the intracellular signaling domain comprises a CD3 zeta intracellular signaling domain.

II.C.2. T Cell Receptor-Engineered (TCR) Cells

In some aspects, an immune cell, e.g., a T cell and/or an NK cell, disclosed herein comprises a T cell receptor (TCR), e.g., an engineered TCR. In some aspects, the TCR specifically binds to a tumor antigen. As used herein, the term “engineered TCR” or “engineered T-cell receptor” refers to a T-cell receptor (TCR) engineered to specifically bind with a desired affinity to a major histocompatibility complex (MHC)/peptide target antigen that is selected, cloned, and/or subsequently introduced into a population of immune cells, e.g., T cells, NK cells, and/or TILs. In some aspects, the TCR specifically binds a neoantigen identified from a cancer patient.

In some aspects, the TCR specifically binds (i.e., targets) one or more antigens expressed on a tumor cell, such as a malignant B cell, a malignant T cell, or a malignant plasma cell. In some aspects, the TCR specifically binds a tumor antigen/MHC complex. In some aspects, the tumor antigen is derived from AFP, CD19, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3F, CD4, CD5, CD7, the extracellular portion of the APRIL protein, neoantigen, or any combinations thereof. In some aspects, the TCR specifically binds (i.e., targets) a tumor antigen derived from NY-ESO-1.

In certain aspects, an engineered cell of the present disclosure can express a T cell receptor (TCR) targeting an antigen. In some aspects, the TCR engineered cells can target main types: shared tumor-associated antigens (shared TAAs) and unique tumor-associated antigens (unique TAAs), or tumor-specific antigens. The former can include, without any limitation, cancer-testis (CT) antigens, overexpressed antigens, and differentiation antigens, while the latter can include, without any limitation, neoantigens and oncoviral antigens. Human papillomavirus (HPV) E6 protein and HPV E7 protein belong to the category of oncoviral antigens.

In some aspects, the TCR engineered cells can target a CT antigen, e.g., melanoma-associated antigen (MAGE) including, but not limited to, MAGE-A1, MAGE-A2, MAGE-A3, MAGE-A4, MAGE-A6, MAGE-A8, MAGE-A9.23, MAGE-A10, and MAGE-A12. In some aspects, the TCR engineered cells can target glycoprotein (gp100), melanoma antigen recognized by T cells (MART-1), and/or tyrosinase, which are mainly found in melanomas and normal melanocytes. In some aspects, the TCR engineered cells can target Wilms tumor 1 (WT1), i.e., one kind of overexpressed antigen that is highly expressed in most acute myeloid leukemia (AML), acute lymphoid leukemia, almost every type of solid tumor and several critical tissues, such as heart tissues. In some aspects, the TCR engineered cells can target mesothelin, another kind of overexpressed antigen that is highly expressed in mesothelioma but is also present on mesothelial cells of several tissues, including trachea.

In some aspects, the TCR engineered cells can target any neoantigen, which can be formed by random somatic mutations specific to individual tumors. In some aspects, the TCR specifically binds to (i.e., targets) a cancer antigen selected from the group consisting of AFP, Braf, CD19, TRAC, TCRβ, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA4, CD20, folate receptor alpha, ERBB2 (Her2/neu), MUC1, MUC16, EGFR, NCAM, prostase, PAP, ELF2M, Ephrin B2, IGF-1 receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, or any combinations thereof.

In certain aspects, the TCR specifically binds (i.e., targets) hTERT. In some aspects, the TCR specifically binds (i.e., targets) KRAS. In some aspects, the TCR specifically binds (i.e., targets) Braf. In some aspects, the TCR specifically binds (i.e., targets) TGFβRII. In some aspects, the TCR specifically binds (i.e., targets) MAGE A10/A4. In some aspects, the TCR specifically binds (i.e., targets) AFP. In some aspects, the TCR specifically binds (i.e., targets) PRAME. In some aspects, the TCR specifically binds (i.e., targets) MAGE A1. In some aspects, the TCR specifically binds (i.e., targets) WT-1. In some aspects, the TCR specifically binds (i.e., targets) NY-ESO. In some aspects, the TCR specifically binds (i.e., targets) PRAME. In some aspects, the TCR specifically binds (i.e., targets) NY-ESO. In some aspects, the TCR specifically binds (i.e., targets) CD19. In certain aspects, the TCR specifically binds a neoantigen identified from a cancer patient.

In some aspects, the TCR comprises an intracellular gamma/delta domain. In some aspects, the TCR is an antibody-T-cell receptor (AbTCR) (see, e.g., Xu et al., Cell Discovery 4:62 (2018), which is incorporated by reference herein in its entirety.

II.C.3. T Cell Receptor Mimics (TCRm)

In some aspects, an immune cell, e.g., a T cell and/or an NK cell, disclosed herein comprises a T cell receptor mimic (TCRm), also known as a TCR-like antibody. TCRm are a type of antibody that recognize epitopes comprising both the peptide and the MHC-I molecule, similar to the recognition of such complexes by the TCR on T cells (see, e.g., Traneska et al., Front. Immunol. 8(1001):1-12 (2017), which is incorporated by reference herein in its entirety). In some aspects, the TCRm specifically binds to a tumor antigen. In certain aspects, the TCRm specifically binds a neoantigen identified from a cancer patient.

In some aspects, the TCRm specifically binds (i.e., target) one or more antigens expressed on a tumor cell, such as a malignant B cell, a malignant T cell, or a malignant plasma cell. In some aspects, the TCRm is a monoclonal antibody. In some aspects, the TCRm specifically binds to WT1. In some aspects, the TCRm specifically binds to a fragment of WT1. In some aspects, the TCRm comprises ESK1 (see, e.g., Ataie et al., J. Mol. Biol. 428(1):194-205 (2016), which is incorporated by reference herein in its entirety). In some aspects, the TCRm specifically binds to MAGE-A1. In some aspects, the TCRm specifically binds to p68 RNA helicase/HLA-A*02:01. In some aspects, the TCRm specifically binds to hCG-b/HLAA*02:01. In some aspects, the TCRm specifically binds to Her2-E75/HLA-A*02:01. In some aspects, the TCRm specifically binds to PR-1 in context of HLA-A*02:01 (see, e.g., Oncoimmunology 5(1): e1049803 (June 2015), which is incorporated by reference herein in its entirety). In some aspects, the TCRm specifically binds to the survivin-2B-derived nonamer peptide, AYACNTSTL (SV2B80-88), presented on HLA-A*24 (SV2B80-88/HLA-A*24) (see, e.g., Kurosawa et al., Nature Scientific Reports 9(9827):1-11 (2019), which is incorporated by reference herein in its entirety). In some aspects, the TCRm specifically binds one or more tumor-associated PRAME peptide/HLA-I antigens (see, e.g., J Clin Invest. 127(7):2705-18 (2017), which is incorporated by reference herein in its entirety). In some aspects, the TCRm specifically binds to tyrosinase. In some aspects, the TCRm specifically binds telomerase catalytic subunit. In some aspects, the TCRm specifically binds to glycoprotein 100 (gp100). In some aspects, the TCRm specifically binds to mucin 1 (MUC1). In some aspects, the TCRm specifically binds to human telomerase reverse transcriptase (hTERT). In some aspects, the TCRm specifically binds to NYESO-1. In some aspects, the TCRm specifically binds to MART-1. In some aspects, the TCRm specifically binds to PRAME.

In some aspects, the TCRm specifically binds to a viral antigen. In some aspects, the TCRm specifically binds to Env183/A2 (Hep B/HLA-A*02:01). In some aspects, the TCRm specifically binds to KP14/1 and KP15/11 (HIV envelope gp160/HLAA*02:01). In some aspects, the TCRm specifically binds to RL36A (West Nile Virus/mouse H-2db). In some aspects, the TCRm specifically binds to a viral epitope derived from HTLV. In some aspects, the TCRm specifically binds to a viral epitope derived from influenza. In some aspects, the TCRm specifically binds to a viral epitope derived from CMV. In some aspects, the TCRm specifically binds to a viral epitope derived from HIV.

II.C.4. c-Jun Polypeptides

In some aspects, immune cells described herein (e.g., cultured using the methods provided herein) comprise, or are capable of overexpressing, a c-Jun polypeptide. In some cases, expression of the endogenous c-Jun protein is induced thereby resulting in increased or overexpression of the c-Jun polypeptide. In some aspects, an immune cell disclosed herein, e.g., a T cell and/or an NK cell, is engineered or modified with a transcription activator (e.g., CRISPR/Cas system-based), wherein the transcription activator is capable of inducing and/or increasing the endogenous expression of a c-Jun polypeptide. In some aspects, the c-Jun polypeptide is exogenously added to the cell (wild type human c-Jun available at GenBank under accession number AAA59197.1 or at UniProtKB (under accession number P05412.2). In some aspects, the c-Jun polypeptide is recombinantly expressed in the immune cell (e.g., T cell and/or NK cell). In some aspects, a c-Jun polypeptide is overexpressed in an immune cell (e.g., T cell and/or NK cell) that has been engineered to express a CAR, TCR, TCR mimic, or other transgene as described herein. Thus, in some aspects the immune cells (e.g., T cells and/or NK cells) described herein (e.g., cultured using the methods provided herein) express a higher level (e.g., at least 10, 20, 30, 40, 50, 60, 70, 80, 90, or 100% more, or at least 1.5-, 2-, 3-, 4-, 5-, or 10-fold more) of c-Jun polypeptide than corresponding cells that have not been modified to overexpress a c-Jun polypeptide. In some aspects, the engineered cells express at least about 2-100 fold more, about 5-50 fold more, about 5-40 fold more, about 5-30 fold more, about 5-20 fold more, about 8-20 fold more, or about 10-20 fold more c-Jun polypeptide than a reference cell. Overexpression of c-Jun renders CAR T cells less susceptible to exhaustion and thus enhances both anti-tumor efficacy and persistence/expansion in various heme and solid tumor models (Lynn et al., Nature 2019, 576:293-300).

### III. Compositions of the Disclosure

Certain aspects of the present disclosure are directed to a cell composition comprising a population of immune cells (e.g., T cell and/or NK cell) cultured according to the methods disclosed herein. Cell populations cultured according to the methods and/or in a metabolic reprogramming medium disclosed herein have an increased number of less-differentiated cells as compared to comparable cells cultured according to conventional methods, e.g., in media containing less than 5 mM K⁺. In some aspects, the cells cultured according to the methods disclosed herein exhibit increased expression of one or more marker typical of a stem-like phenotype. In some aspects, cell populations cultured according to the methods and/or in a metabolic reprogramming medium disclosed herein have an increased number of effector-like cells as compared to comparable cells cultured according to conventional methods, e.g., in media containing less than 5 mM K⁺. In some aspects, cell populations cultured according to the methods and/or in a metabolic reprogramming medium disclosed herein have both an increased number of stem-like and effector-like cells as compared to comparable cells cultured according to conventional methods, e.g., in media containing less than 5 mM K⁺. In some aspects, the cells cultured according to the methods disclosed herein exhibit greater proliferative potential compared to cells cultured according to conventional methods. In some aspects, the cells cultured according to the methods disclosed herein exhibit increased transduction efficiency. In some aspects, the cells cultured according to the methods disclosed herein exhibit increased in vivo viability upon transplantation in a subject. In some aspects, the cells cultured according to the methods disclosed herein exhibit increased cell potency. In some aspects, the cells cultured according to the methods disclosed herein exhibit decreased cell exhaustion. In some aspects, the cells cultured according to the methods disclosed herein exhibit increased in vivo persistence upon transplantation in a subject. In some aspects, the cells cultured according to the methods disclosed herein exhibit increased in vivo activity upon transplantation in a subject. In some aspects, the cells cultured according to the methods disclosed herein exhibit a more durable in vivo response upon transplantation in a subject. In some aspects, the subject is a human.

In some aspects, at least about 5% of the cells in the cell composition have a stem-like phenotype. In some aspects, at least about 10% of the cells in the cell composition have a stem-like phenotype. In some aspects, at least about 15% of the cells in the cell composition have a stem-like phenotype. In some aspects, at least about 20% of the cells in the cell composition have a stem-like phenotype. In some aspects, at least about 25% of the cells in the cell composition have a stem-like phenotype. In some aspects, at least about 30% of the cells in the cell composition have a stem-like phenotype. In some aspects, at least about 35% of the cells in the cell composition have a stem-like phenotype. In some aspects, at least about 40% of the cells in the cell composition have a stem-like phenotype. In some aspects, at least about 45% of the cells in the cell composition have a stem-like phenotype. In some aspects, at least about 50% of the cells in the cell composition have a stem-like phenotype. In some aspects, at least about 55% of the cells in the cell composition have a stem-like phenotype. In some aspects, at least about 60% of the cells in the cell composition have a stem-like phenotype. In some aspects, at least about 65% of the cells in the cell composition have a stem-like phenotype. In some aspects, at least about 70% of the cells in the cell composition have a stem-like phenotype.

In some aspects, following culture of T cells according to the methods disclosed herein, stem-like T cells constitute at least about 10% to at least about 70% of the total number of T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD8⁺ T cells in the culture. In some aspects, following culture of T cells according to the methods disclosed herein, stem-like T cells constitute at least about 10%, at least about 20%, at least about 30%, at least about 40%, at least about 50%, at least about 60%, or at least about 70% of the total number of CD4⁺ T cells in the culture.

In some aspects, the number of cells having a stem-like phenotype in the cell composition is increased at least about 1.5-fold as compared to the number of cells in the cell composition prior to the culture. In some aspects, the number of cells having a stem-like phenotype in the cell composition is increased at least about 2.0-fold as compared to the number of cells in the cell composition prior to the culture. In some aspects, the number of cells having a stem-like phenotype in the cell composition is increased at least about 2.5-fold as compared to the number of cells in the cell composition prior to the culture. In some aspects, the number of cells having a stem-like phenotype in the cell composition is increased at least about 3.0-fold as compared to the number of cells in the cell composition prior to the culture. In some aspects, the number of cells having a stem-like phenotype in the cell composition is increased at least about 3.5-fold as compared to the number of cells in the cell composition prior to the culture. In some aspects, the number of cells having a stem-like phenotype in the cell composition is increased at least about 4.0-fold as compared to the number of cells in the cell composition prior to the culture. In some aspects, the number of cells having a stem-like phenotype in the cell composition is increased at least about 4.5-fold as compared to the number of cells in the cell composition prior to the culture. In some aspects, the number of cells having a stem-like phenotype in the cell composition is increased at least about 5.0-fold as compared to the number of cells in the cell composition prior to the culture. In some aspects, the number of cells having a stem-like phenotype in the cell composition is increased at least about 5.5-fold as compared to the number of cells in the cell composition prior to the culture. In some aspects, the number of cells having a stem-like phenotype in the cell composition is increased at least about 6.0-fold as compared to the number of cells in the cell composition prior to the culture. In some aspects, the number of cells having a stem-like phenotype in the cell composition is increased at least about 6.5-fold as compared to the number of cells in the cell composition prior to the culture. In some aspects, the number of cells having a stem-like phenotype in the cell composition is increased at least about 7.0-fold as compared to the number of cells in the cell composition prior to the culture. In some aspects, the number of cells having a stem-like phenotype in the cell composition is increased at least about 7.5-fold as compared to the number of cells in the cell composition prior to the culture. In some aspects, the number of cells having a stem-like phenotype in the cell composition is increased at least about 8.0-fold as compared to the number of cells in the cell composition prior to the culture. In some aspects, the number of cells having a stem-like phenotype in the cell composition is increased at least about 9.0-fold as compared to the number of cells in the cell composition prior to the culture. In some aspects, the number of cells having a stem-like phenotype in the cell composition is increased at least about 10-fold as compared to the number of cells in the cell composition prior to the culture. In some aspects, the number of cells having a stem-like phenotype in the cell composition is increased at least about 15-fold as compared to the number of cells in the cell composition prior to the culture. In some aspects, the number of cells having a stem-like phenotype in the cell composition is increased at least about 20-fold as compared to the number of cells in the cell composition prior to the culture. In some aspects, the number of cells having a stem-like phenotype in the cell composition is increased at least about 30-fold as compared to the number of cells in the cell composition prior to the culture. In some aspects, the number of cells having a stem-like phenotype in the cell composition is increased at least about 40-fold as compared to the number of cells in the cell composition prior to the culture. In some aspects, the number of cells having a stem-like phenotype in the cell composition is increased at least about 50-fold as compared to the number of cells in the cell composition prior to the culture. In some aspects, the number of cells having a stem-like phenotype in the cell composition is increased at least about 75-fold as compared to the number of cells in the cell composition prior to the culture. In some aspects, the number of cells having a stem-like phenotype in the cell composition is increased at least about 100-fold as compared to the number of cells in the cell composition prior to the culture. In some aspects, the number of cells having a stem-like phenotype in the cell composition is increased at least about 500-fold as compared to the number of cells in the cell composition prior to the culture. In some aspects, the number of cells having a stem-like phenotype in the cell composition is increased at least about 1000-fold as compared to the number of cells in the cell composition prior to the culture.

In some aspects, following culture of T cells according to the methods disclosed herein, at least about 10% to at least about 70% of the total number of T cells in the culture are CD39⁻/TCF7⁺ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/TCF7+ T cells. In some aspects the T cells are CD4⁺ T cells. In some aspects the T cells are CD8⁺ T cells.

In some aspects, the cell composition comprises immune cells, e.g., T cells and/or NK cells. In some aspects, the cell composition comprises an increase in the percent of immune cells, e.g., T cells and/or NK cells, which express CD95. In some aspects, the cell composition comprises an increase in the percent of immune cells, e.g., T cells and/or NK cells, which do not express CD45RO. In some aspects, the cell composition comprises an increase in the percent of immune cells, e.g., T cells and/or NK cells, which express CD45RA. In some aspects, the cell composition comprises an increase in the percent of immune cells, e.g., T cells and/or NK cells, which express CCR7. In some aspects, the cell composition comprises an increase in the percent of immune cells, e.g., T cells and/or NK cells, which express CD62L. In some aspects, the cell composition comprises an increase in the percent of immune cells, e.g., T cells and/or NK cells, which express TCF7. In some aspects, the cell composition comprises an in the increase percent of immune cells, e.g., T cells and/or NK cells, which express CD3. In some aspects, the cell composition comprises an increase in the percent of immune cells, e.g., T cells and/or NK cells, which express CD27. In some aspects, the cell composition comprises an in the increase percent of immune cells, e.g., T cells and/or NK cells, which express CD95 and CD45RA. In some aspects, the cell composition comprises an increase in the percent of immune cells, e.g., T cells and/or NK cells, which express CD45RA and CCR7. In some aspects, the cell composition comprises an increase in the percent of immune cells, e.g., T cells and/or NK cells, which express CD95, CD45RA, and CCR7. In some aspects, the cell composition comprises an increase in the percent of immune cells, e.g., T cells and/or NK cells, which express CD45RA, CCR7, and CD62L. In some aspects, the cell composition comprises an increase in the percent of immune cells, e.g., T cells and/or NK cells, which express CD95, CD45RA, CCR7, and CD62L. In some aspects, the cell composition comprises an increase in the percent of immune cells, e.g., T cells and/or NK cells, which express CD45RA, CCR7, CD62L, and TCF7. In some aspects, the cell composition comprises an increase in the percent of immune cells, e.g., T cells and/or NK cells, which express CD95, CD45RA, CCR7, CD62L, and TCF7. In some aspects, the cell composition comprises an increase in the percent of immune cells, e.g., T cells and/or NK cells, which express CD45RA, CCR7, CD62L, TCF7, and CD27. In some aspects, the cell composition comprises an increase in the percent of immune cells, e.g., T cells and/or NK cells, which express CD95, CD45RA, CCR7, CD62L, TCF7, and CD27. In some aspects, the cell composition comprises an increase in the percent of immune cells, e.g., T cells and/or NK cells, which express, CD45RA, CCR7, CD62L, TCF7, and CD27, and which do not express CD45RO or which are CD45RO^{low}. In some aspects, the cell composition comprises an increase in the percent of immune cells, e.g., T cells and/or NK cells, which express CD95, CD45RA, CCR7, CD62L, TCF7, and CD27, and which do not express CD45RO or which are CD45RO^{low}.

In some aspects, the cell composition comprises an increase in the percent of immune cells, e.g., T cells and/or NK cells, which do not express CD39 and CD69. In some aspects, the cell composition comprises an increase in the percent of immune cells, e.g., T cells and/or NK cells, which express CD8, and which do not express CD39 and CD69. In some aspects, following culture of T cells according to the methods disclosed herein, at least about 10% to at least about 40% of the total number of T cells in the culture are CD39⁻/CD69⁻ T cells. In some aspects, following culture of T cells according to the methods disclosed herein, at least about 10%, at least about 15%, at least about 20%, at least about 25%, at least about 30%, at least about 35%, or at least about 40% of the total number of T cells in the culture are CD39⁻/CD69⁻ T cells.

In some aspects, the cell composition comprises an increased percentage of immune cells, e.g., T cells and/or NK cells, which express both (i) one or more stem-like markers and (ii) one or more effector-like markers. In some aspects, the cell composition comprises an increased percentage of immune cells, e.g., T cells and/or NK cells, which express at least two stem-like markers and one or more effector-like markers. In some aspects, the cell composition comprises an increase percent of immune cells, e.g., T cells and/or NK cells, which express at least three stem-like markers and one or more effector-like markers. In some aspects, the cell composition comprises an increased percentage of immune cells, e.g., T cells and/or NK cells, which express at least four stem-like markers and one or more effector-like markers. In some aspects, the cell composition comprises an increased percentage of immune cells, e.g., T cells and/or NK cells, which express one or more stem-like markers and at least two effector-like markers.

In some aspects, the stem-like markers are selected from CD45RA+, CD62L+, CCR7+, CD27+, CD28+, BACH2+, LEF1+, TCF7+, and any combination thereof. In some aspects the stem-like markers comprise CD45RA+, CD62L+, CCR7+, and TCF7+, or any combination thereof. In some aspects, the cell expresses CD45RO^{low}. In some aspects, the stem-like markers comprise one or more genes listed herein as part of a gene-signature (see supra; see, e.g., Gattinoni, L., et al., Nat Med 17(10): 1290-97 (2011) or Galletti et al. Nat Immunol 21, 1552-62 (2020)).

In some aspects, the stem-like markers comprise a gene expressed in the WNT signaling pathway. In some aspects, the stem-like markers comprise one or more genes selected from GNG2, PSMC3, PSMB10, PSMC5, PSMB8, PSMB9, AKT1, MYC, CLTB, PSME1, DVL2, PFN1, H2AFJ, LEF1, CTBP1, MOV10, HIST1H2BD, FZD3, ITPR3, PARD6A, LRP5, HIST2H4A, HIST2H3C, HIST1H2AD, HIST2H2BE, HIST3H2BB, DACT1, and any combination thereof. In some aspects, the stem-like markers comprise one or more genes selected from MYC, AKT1, LEF1, and any combination thereof.

In some aspects, the effector-like markers are selected from pSTAT5+, STAT5+, pSTAT3+, STAT3+, and any combination thereof. In some aspects, the effector-like marker comprises a STAT target selected from the group consisting of AKT1, AKT2, AKT3, BCL2L1, CBL, CBLB, CBLC, CCND1, CCND2, CCND3, CISH, CLCF1, CNTF, CNTFR, CREBBP, CRLF2, CSF2, CSF2RA, CSF2RB, CSF3, CSF3R, CSH1, CTF1, EP300, EPO, EPOR, GH1, GH2, GHR, GRB2, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNLR1, IFNW1, IL10, IL10RA, IL10RB, IL11, IL11RA, IL12A, IL12B, IL12RB1, IL12RB2, IL13, IL13RA1, IL13RA2, IL15, IL15RA, IL19, IL2, IL20, IL20RA, IL20RB, IL21, IL21R, IL22, IL22RA1, IL22RA2, IL23A, IL23R, IL24, IL26, IL2RA, IL2RB, IL2RG, IL3, IL3RA, IL4, IL4R, IL5, IL5RA, IL6, IL6R, IL6ST, IL7, IL7R, IL9, IL9R, IRF9, JAK1, JAK2, JAK3, LEP, LEPR, LIF, LIFR, MPL, MYC, OSM, OSMR, PIAS1, PIAS2, PIAS3, PIAS4, PIK3CA, PIK3CB, PIK3CD, PIK3CG, PIK3R1, PIK3R2, PIK3R3, PIK3R5, PIM1, PRL, PRLR, PTPN11, PTPN6, SOCS1, SOCS2, SOCS3, SOCS4, SOCS5, SOCS7, SOS1, SOS2, SPRED1, SPRED2, SPRY1, SPRY2, SPRY3, SPRY4, STAM, STAM2, STAT1, STAT2, STAT3, STAT4, STAT5A, STAT5B, STAT6, TPO, TSLP, TYK2, and any combination thereof.

In some aspects, the effector-like markers are effector memory-associated genes that comprise one or more genes selected from TBCD, ARL4C, KLF6, LPGAT1, LPIN2, WDFY1, PCBP4, PIK343, FAS, LLGL2, PPP2R2B, TTC39C, GGA2, LRP8, PMAIP1, MVD, IL15RA, FHOD1, EML4, PEA15, PLEKHA5, WSB2, PAM, CD68, MSC, TLR3, S1PR5, KLRB1, CYTH3, RAB27B, SCD5, and any combination thereof. In some aspects, the effector-like markers comprise one or more genes selected from KLF6, FAS, KLRB1, TLR3, and any combination thereof.

In some aspects, the cell composition comprises an increase in the percent of immune cells, e.g., T cells and/or NK cells, that are CD45RA+, STAT5+, and STAT3+. In some aspects, the cell composition comprises an increase in the percent of immune cells e.g., T cells and/or NK cells, that are CD62L+, STAT5+, and STAT3+. In some aspects, the cell composition comprises an increase in the percent of immune cells, e.g., T cells and/or NK cells, that are TCF7+, STAT5+, and STAT3+. In some aspects, the cell composition comprises an increase in the percent of immune cells, e.g., T cells and/or NK cells, that are CD45RA+, CD62L+, CCR7+, CD27+, CD28+, BACH2+, LEF1+, TCF7+, STAT5+, and STAT3+. In some aspects, the cell composition comprises an increase in the percent of immune cells, e.g., T cells and/or NK cells, that are CD45RA+, CD62L+, CCR7+, CD27+, CD28+, BACH2+, LEF1+, TCF7+, pSTAT5+, STAT5+, pSTAT3+, and STAT3+. In some aspects, the cell composition comprises an increase in the percent of immune cells, e.g., T cells and/or NK cells, that are CD45RA+, CD45RO−, CD62L+, CCR7+, CD27+, CD28+, BACH2+, LEF1+, TCF7+, pSTAT5+, STAT5+, pSTAT3+, and STAT3+.

In some aspects, an immune cell, e.g., T cells and/or NK cells, comprises one or more markers selected from CD45RA+, CD62L+, CCR7+, CD27+, CD28+, BACH2+, LEF1+, TCF7+, and any combination thereof and one or more markers selected from pSTAT5+, STAT5+, pSTAT3+, STAT3+, and any combination thereof. In some aspects, the immune cell, e.g., T cells and/or NK cells, expresses CD45RO^{low}. In some aspects, an immune cell, e.g., T cells and/or NK cells, comprises one or more markers selected from CD45RA+, CD62L+, CCR7+, CD27+, CD28+, BACH2+, LEF1+, TCF7+, and any combination thereof and one or more effector-like markers. In some aspects, an immune cell, e.g., T cells and/or NK cells, comprises one or more stem-like markers and one or more markers selected from pSTAT5+, STAT5+, pSTAT3+, STAT3+, and any combination thereof. In some aspects, the immune cell, e.g., T cells and/or NK cells, expresses CD45RO^{low}.

Some aspects of the present disclosure are directed to a cell composition comprising a population of immune cells, wherein the population of immune cells comprises (i) a first sub-population of immune cells expressing one or more stem-like markers (e.g., stem-like immune cells) and (ii) a second sub-population of immune cells expressing one or more effector-like marker (e.g., effector-like immune cells), wherein the population of immune cells comprises a higher percentage (i.e., the number of stem-like immune cells/the total number of immune cells) of the first sub-population of immune cells expressing one or more stem-like markers, as compared to a population of immune cells cultured using conventional methods, e.g., in a medium having less than 5 mM potassium ion. In some aspects the immune cells are T cells. In some aspects the immune cells are NK cells. In some aspects, the immune cells, e.g., T cells and/or NK cells, cultured according to the methods disclosed herein result in these cell compositions.

In some aspects, immune cells, e.g., T cells and/or NK cells, cultured according to the methods disclosed herein have increased expression, e.g., a higher percentage of immune cells, e.g., T cells and/or NK cells, that express, GZMB, MHC-II, LAG3, TIGIT, and/or NKG7, and decreased expression, e.g., a lower percentage of immune cells, e.g., T cells and/or NK cells, that express, IL-32. Cells highest for NKG7 have been shown to be better killers (Malarkannan et al. 2020 Nat. Immuno.), whereas cells higher in IL-32 have been shown to have activation-induced cell death (Goda et al., 2006 Int. Immunol). In some aspects the immune cells, e.g., T cells and/or NK cells, with higher expression of GZMB, MHC-II, LAG3, TIGIT, and/or NKG7 are CD8+ T cells expressing effector-like markers. In some aspects the immune cells, e.g., T cells and/or NK cells, with lower expression of IL-32 are CD8+ T cells expressing effector-like markers.

In some aspects, the cell composition comprises one or more immune cell, e.g., T cells, NK cells, and/or TILs, which is genetically engineered. In some aspects, the cell composition comprises one or more immune cell, e.g., T cells, NK cells, and/or TILs, which is engineered to express a chimeric antigen receptor (CAR). Any CAR disclosed herein, e.g., in section II.G.1, above, can be used in the cells of the cell composition.

In some aspects, the cell composition comprises one or more immune cell, e.g., T cells, NK cells, and/or TILs, which is engineered to express a T cell receptor (TCR), e.g., an engineered TCR. Any TCR disclosed herein, e.g., in section II.C.2, below, can be used in the cells of the cell composition.

In some aspects, the cell composition comprises one or more immune cell, e.g., T cells, NK cells, and/or TILs, which is engineered to express a TCRm. Any TCRm disclosed herein, e.g., in section II.C.3, below, can be used in the cells of the cell composition.

In some aspects, the cell composition, obtained by any method described herein (e.g., the yield of the final cell product for use as a therapy), comprises at least about 1×10⁵, 5×10⁵, 1×10⁶, 5×10⁶, 1×10⁷, 5×10⁷, 1×10⁸, 5×10⁸, 1×10⁹, or 5×10⁹ cells. In some aspects, the cell composition, obtained by any method described herein, comprises at least about 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, or 5×10⁹ stem-like cells. In some aspects, the cell composition, obtained by any method described herein, comprises at least about 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 10×10¹⁰, 11×10¹⁰, 12×10¹⁰, 13×10¹⁰, 14×10¹⁰, or 15×10¹⁰ cells. In some aspects, the cell composition, obtained by any method described herein, comprises at least about 1×10⁶ cells. In some aspects, the cell composition, obtained by any method described herein, comprises at least about 1×10⁶ stem-like cells. In some aspects, the cell composition, obtained by any method described herein, comprises at least about 1×10¹⁰ cells. In some aspects, the cell composition, obtained by any method described herein, comprises at least about 2×10¹⁰ cells. In some aspects, the cell composition, obtained by any method described herein, comprises at least about 3×10¹⁰ cells. In some aspects, the cell composition, obtained by any method described herein, comprises at least about 4×10¹⁰ cells. In some aspects, the cell composition, obtained by any method described herein, comprises at least about 5×10¹⁰ cells. In some aspects, the cell composition, obtained by any method described herein, comprises at least about 6×10¹⁰ cells. In some aspects, the cell composition, obtained by any method described herein, comprises at least about 7×10¹⁰ cells. In some aspects, the cell composition, obtained by any method described herein, comprises at least about 8×10¹⁰ cells. In some aspects, the cell composition, obtained by any method described herein, comprises at least about 9×10¹⁰ cells. In some aspects, the cell composition, obtained by any method described herein, comprises at least about 10×10¹⁰ cells. In some aspects, the cell composition, obtained by any method described herein, comprises at least about 11×10¹⁰ cells. In some aspects, the cell composition, obtained by any method described herein, comprises at least about 12×10¹⁰ cells. In some aspects, the cell composition, obtained by any method described herein, comprises at least about 13×10¹⁰ cells. In some aspects, the cell composition, obtained by any method described herein, comprises at least about 14×10¹⁰ cells. In some aspects, the cell composition, obtained by any method described herein, comprises at least about 15×10¹⁰ cells. In some aspects, cell yield represents the total number of CD3+ cells.

In some aspects, the methods disclosed herein yield a composition comprising at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ cells by at least about day 10 of culturing in the presently disclosed medium. In some aspects, the methods disclosed herein yield a composition comprising at least about 1.8×10¹⁰ cells by at least about day 10 of culturing in the presently disclosed medium.

In some aspects, the cell composition comprises at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ stem-like cells. In some aspects, the methods disclosed herein yield a composition comprising at least about 1×10¹⁰, at least about 1.1×10¹⁰, at least about 1.2×10¹⁰, at least about 1.3×10¹⁰, at least about 1.4×10¹⁰, at least about 1.5×10¹⁰, at least about 1.6×10¹⁰, at least about 1.7×10¹⁰, at least about 1.8×10¹⁰, at least about 1.9×10¹⁰, or at least about 2.0×10¹⁰ stem-like cells by at least about day 10 of culture. In some aspects, the methods disclosed herein yield a composition comprising at least about 1.8×10¹⁰ stem-like cells by at least about day 10 of culturing in the presently disclosed medium.

In some aspects, the methods disclosed herein yield a composition comprising immune cells that are at least about 80%, at least about 85%, at least about 90%, at least about 94%, at least about 95%, at least about 96%, at least about 97%, at least about 98%, or at least about 99% viable. In some aspects, the methods disclosed herein yield a composition comprising at least about 1.8×10¹⁰ stem-like cells with at least about 94% cell viability.

### IV. Methods of Treatment

Some aspects of the present disclosure are directed to methods of treating a subject in need thereof comprising administering to the subject a population of immune cells, e.g., T cells and/or NK cells, cultured according to the methods disclosed herein (e.g., in a medium comprising potassium ion at a concentration higher than 5 mM).

The present disclosure also provides methods of stimulating a T cell-mediated immune response to a target cell population or tissue in a subject, comprising administering an effective amount of a population of immune cells, e.g., T cells and/or NK cells, cultured according to the methods disclosed herein (e.g., in a medium comprising potassium ion at a concentration higher than 5 mM).

The present disclosure also provides methods of providing an anti-tumor immunity in a subject in need thereof, comprising administering a population of immune cells, e.g., T cells and/or NK cells, cultured according to the methods disclosed herein (e.g., in a medium comprising potassium ion at a concentration higher than 5 mM).

In some aspects, the population of immune cells administered comprises T cells. In some aspects, the T cells are autologous T cells. In some aspects the T cells are allogeneic T cells. In some aspects, the T cells comprise a CAR, i.e., CAR-T cells, as described herein. In some aspects, the T cells comprise a heterologous TCR, as described herein. In some aspects, the T cells comprise an engineered TCR, as described herein. In some aspects, the T cells comprise an TCR mimic, as described herein.

In some aspects, the subject is afflicted with a cancer, e.g., a tumor. In some aspects, administering the population of immune cells cultured according to the methods disclosed herein (e.g., by contacting the immune cells with PCS in a medium comprising potassium ion at a concentration higher than 5 mM) reduces a tumor volume in the subject compared to a reference tumor volume. In some aspects, the reference tumor volume is the tumor volume in the subject prior to the administration of the engineered cell. In further aspects, the reference tumor volume is the tumor volume in a corresponding subject that did not receive the administration. In some aspects, the tumor volume in the subject is reduced by at least about 5%, at least about 10%, at least about 15%, at least about 30%, at least about 40%, at least about 50%, at least about 60%, at least about 70%, at least about 80%, at least about 90%, or at least about 100% after the administration compared to the reference tumor volume.

In some aspects, treating a tumor comprises reducing a tumor weight in the subject. In certain aspects, administering the population of immune cells cultured according to the methods disclosed herein (e.g., by contacting the immune cells with PCS in a medium comprising potassium ion at a concentration higher than 5 mM) reduces the tumor weight in a subject when administered to the subject. In some aspects, the tumor weight is reduced by at least about 5%, at least about 10%, at least about 15%, at least about 30%, at least about 40%, at least about 50%, at least about 60%, at least about 70%, at least about 80%, at least about 90%, or at least about 100% after the administration compared to a reference tumor weight. In some aspects, the reference tumor weight is the tumor weight in the subject prior to the administration of the population of immune cells of the disclosure. In further aspects, the reference tumor weight is the tumor weight in a corresponding subject that did not receive the administration.

In some aspects, administering the population of immune cells cultured according to the methods disclosed herein (e.g., by contacting the immune cells with PCS in a medium comprising potassium ion at a concentration higher than 5 mM) to a subject, e.g., suffering from a tumor, increases the number and/or percentage of TILs (e.g., CD4⁺ or CD8⁺) in a tumor and/or a tumor microenvironment (TME) of the subject. In certain aspects, the number and/or percentage of TILs in a tumor and/or TME is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, at least about 200%, at least about 210%, at least 220%, at least about 230%, at least about 240%, at least about 250%, at least about 260%, at least about 270%, at least about 280%, at least about 290%, or at least about 300% or more compared to a reference (e.g., corresponding value in a subject that did not receive the cell composition of the present disclosure or the same subject prior to the administration of the cell composition of the present disclosure).

In some aspects, administering the population of immune cells cultured according to the methods disclosed herein (e.g., by contacting the immune cells with PCS in a medium comprising potassium ion at a concentration higher than 5 mM) to a subject, e.g., suffering from a tumor, can increase the duration of an immune response in a subject relative to the duration of an immune response in a subject administered a similar cell therapy comprising cells prepared according to conventional methods, e.g., cultured in a medium not comprising a potassium ion concentration of at least 50 mM. In certain aspects, the duration of the immune response is increased by at least about 10%, at least about 20%, at least about 30%, at least about 40%, at least about 50%, at least about 75%, at least about 100%, at least about 150%, at least about 200%, at least about 300%, at least about 400%, at least about 500%, or at least about 1000% or more compared to a reference (e.g., a subject administered a similar cell therapy comprising cells prepared according to conventional methods, e.g., cultured in a medium not comprising a potassium ion concentration of at least 50 mM). In certain aspects, the duration of the immune response is increased by at least about 2-fold, at least about 3-fold, at least about 4-fold, at least about 5-fold, at least about 6-fold, at least about 7-fold, at least about 8-fold, at least about 9-fold, or at least about 10-fold or more compared to a reference (e.g., a subject administered a similar cell therapy comprising cells prepared according to conventional methods, e.g., cultured in a medium not comprising a potassium ion concentration of at least 50 mM).

In addition to the above, administering the population of immune cells cultured according to the methods disclosed herein (e.g., by contacting the immune cells with PCS in a medium comprising potassium ion at a concentration higher than 5 mM) has other effects which are conducive for the treatment of a tumor.

As described herein, the population of immune cells cultured according to the methods disclosed herein (e.g., by contacting the immune cells with PCS in a medium comprising potassium ion at a concentration higher than 5 mM) can be used to treat variety of cancer types, e.g., a tumor derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

In some aspects, the population of immune cells cultured according to the methods disclosed herein (e.g., by contacting the immune cells with PCS in a medium comprising potassium ion at a concentration higher than 5 mM) can be used in combination with other therapeutic agents (e.g., anti-cancer agents and/or immunomodulating agents). Accordingly, in certain aspects, a method of treating a tumor disclosed herein comprises administering the population of immune cells of the disclosure in combination with one or more additional therapeutic agents.

In some aspects, the population of immune cells cultured according to the methods disclosed herein (e.g., by contacting the immune cells with PCS in a medium comprising potassium ion at a concentration higher than 5 mM) is used in combination with one or more anti-cancer agents, such that multiple elements of the immune pathway can be targeted. In some aspects, an anti-cancer agent comprises an immune checkpoint inhibitor (i.e., blocks signaling through the particular immune checkpoint pathway).

Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. In some aspects, the checkpoint inhibitor is a PD-1 antagonist. In some aspects, the checkpoint inhibitor is an anti-PD-1 antibody. In some aspects, the checkpoint inhibitor is an anti-PD-L1 antibody. A comprehensive and non-limiting list of combination treatment is disclosed in detail elsewhere in this application.

In some aspects, the population of immune cells cultured according to the methods disclosed herein (e.g., by contacting the immune cells with PCS in a medium comprising potassium ion at a concentration higher than 5 mM) is administered to the subject prior to or after the administration of the additional therapeutic agent. In other aspects, the population of immune cells cultured according to the methods disclosed herein is administered to the subject concurrently with the additional therapeutic agent. In certain aspects, the population of immune cells cultured according to the methods disclosed herein and the additional therapeutic agent can be administered concurrently as a single composition in a pharmaceutically acceptable carrier. In other aspects, the population of immune cells of the disclosure and the additional therapeutic agent are administered concurrently as separate compositions.

In some aspects, the subject is a nonhuman animal such as a rat or a mouse. In some aspects, the subject is a human.

In some aspects, the population of immune cells cultured according to the methods disclosed herein (e.g., by contacting the immune cells with PCS in a medium comprising potassium ion at a concentration higher than 5 mM) is used in combination with other therapeutic agents (e.g., anti-cancer agents and/or immunomodulating agents). Accordingly, in certain aspects, a method of treating a tumor disclosed herein comprises administering a population of immune cells of the present disclosure in combination with one or more additional therapeutic agents to a subject. Such agents can include, for example, chemotherapeutic drug, targeted anti-cancer therapy, oncolytic drug, cytotoxic agent, immune-based therapy, cytokine, surgical procedure, radiation procedure, activator of a costimulatory molecule, immune checkpoint inhibitor, a vaccine, a cellular immunotherapy, or any combination thereof.

In some aspects, the population of immune cells cultured according to the methods disclosed herein (e.g., by contacting the immune cells with PCS in a medium comprising potassium ion at a concentration higher than 5 mM) is used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, the population of immune cells cultured according to the methods disclosed herein (e.g., by contacting the immune cells with PCS in a medium comprising potassium ion at a concentration higher than 5 mM) is used in combination with one or more anti-cancer agents, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD-1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically engineered cells, e.g., cells engineered to express a chimeric antigen receptor (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; blocking of immuno repressive cytokines; or any combination thereof.

In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors include the following: anti-PD1 antibody (e.g., nivolumab (OPDIVO®), pembrolizumab (KEYTRUDA®; MK-3475), pidilizumab (CT-011), PDR001, MEDI0680 (AMP-514), TSR-042, REGN2810, JS001, AMP-224 (GSK-2661380), PF-06801591, BGB-A317, BI 754091, SHR-1210, and combinations thereof); anti-PD-L1 antibody (e.g., atezolizumab (TECENTRIQ®; RG7446; MPDL3280A; RO5541267), durvalumab (MEDI4736, IMFINZI®), BMS-936559, avelumab (BAVENCIO®), LY3300054, CX-072 (Proclaim-CX-072), FAZ053, KN035, MDX-1105, and combinations thereof); and anti-CTLA-4 antibody (e.g., ipilimumab (YERVOY®), tremelimumab (ticilimumab; CP-675,206), AGEN-1884, ATOR-1015, and combinations thereof).

In some aspects, an anti-cancer agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), TIM3 agonist (e.g., anti-TIM3 antibody), or combinations thereof.

In some aspects, the population of immune cells cultured according to the methods disclosed herein (e.g., by contacting the immune cells with PCS in a medium comprising potassium ion at a concentration higher than 5 mM) is administered to the subject prior to or after the administration of the additional therapeutic agent. In other aspects, the population of immune cells disclosed herein is administered to the subject concurrently with the additional therapeutic agent. In certain aspects, the population of immune cells disclosed herein and the additional therapeutic agent can be administered concurrently as a single composition in a pharmaceutically acceptable carrier. In other aspects, the population of immune cells disclosed herein and the additional therapeutic agent are administered concurrently as separate compositions. In some aspects, the additional therapeutic agent and the population of immune cells disclosed herein are administered sequentially.

Certain aspects of the present disclosure are directed to methods of treating an autoimmune disease, comprising administering a population of immune cells, e.g., comprising a Treg cell, cultured according to any of the methods disclosed herein. Other aspects of the present disclosure are directed to methods of treating an inflammatory pathology, comprising administering a population of immune cells, e.g., comprising a Treg cell, cultured according to any of the methods disclosed herein. In some aspects, the inflammatory pathology comprises cytokine release syndrome. In some aspects, the inflammatory pathology comprises sepsis. In some aspects, the inflammatory pathology comprises graft-versus host disease. In some aspects, the immune cell, e.g., the Treg cell, is engineered.

### EXAMPLES

### Example 1

To assess the effect of anti-CD3/28 density of PCS products on anti-ROR1 CAR T cell expansion during production in MRM, the following anti-CD3/28 densities of PCS products were prepared according to Table 1.

TABLE 1

| | | OKT3- | CD28.2- | IL2-to- | IL7-to- | IL15-to- |
|---|---|---|---|---|---|---|
| | | to-MSR | to-MSR | MSR | MSR | MSR |
| PCS | PCS Formulation | mass | mass | mass | mass | mass |
| ID | Description | ratio | ratio | ratio | ratio | ratio |
| PCS-1 | 3/28 1:1 0.1% biotin | 0.005 | 0.005 | 0.0013 | 0.0013 | 0.0013 |
| | POPC IL2/7/15 | | | | | |
| | 0.0013:0.0013:0.0013 | | | | | |
| PCS-2 | 3/28 1:1 0.3% biotin | 0.015 | 0.015 | 0.0013 | 0.0013 | 0.0013 |
| | POPC IL2/7/15 | | | | | |
| | 0.0013:0.0013:0.0013 | | | | | |
| PCS-3 | 3/28 1:1 0.5% biotin | 0.025 | 0.025 | 0.0013 | 0.0013 | 0.0013 |
| | POPC IL2/7/15 | | | | | |
| | 0.0013:0.0013:0.0013 | | | | | |
| PCS-4 | 3/28 1:1 0.75% biotin | 0.0375 | 0.0375 | 0.0013 | 0.0013 | 0.0013 |
| | POPC IL2/7/15 | | | | | |
| | 0.0013:0.0013:0.0013 | | | | | |
| PCS-5 | 3/28 1:1 1% biotin | 0.05 | 0.05 | 0.0013 | 0.0013 | 0.0013 |
| | POPC IL2/7/15 | | | | | |
| | 0.0013:0.0013:0.0013 | | | | | |

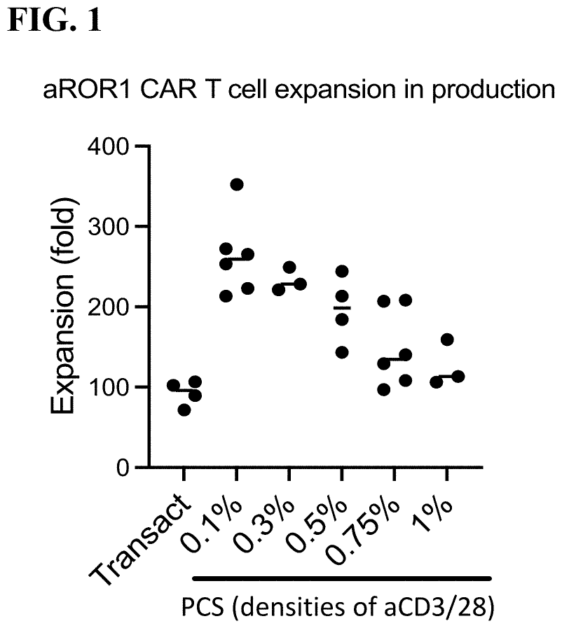

Healthy donor T cells were activated using either TRANSACT™ or PCS formulations with varying anti-CD3/28 density (0.10%-1%; Table 1) in MRM. T cells were transduced with an R12 construct and cultured in MRM for 8 days. On day 8, the T cells were counted and the total expansion from day 0 was determined (FIG. 1). All PCS-activated T cell products showed higher expansion compared to the TRANSACT™-activated T cell product. Lower density PCS formulations produced 2-3 fold more cells when compared to TRANSACT™ activation. Within the PCS products produced in MRM, higher anti-CD3/28 density showed reduced T cell expansion in a density-dependent manner.

### Example 2

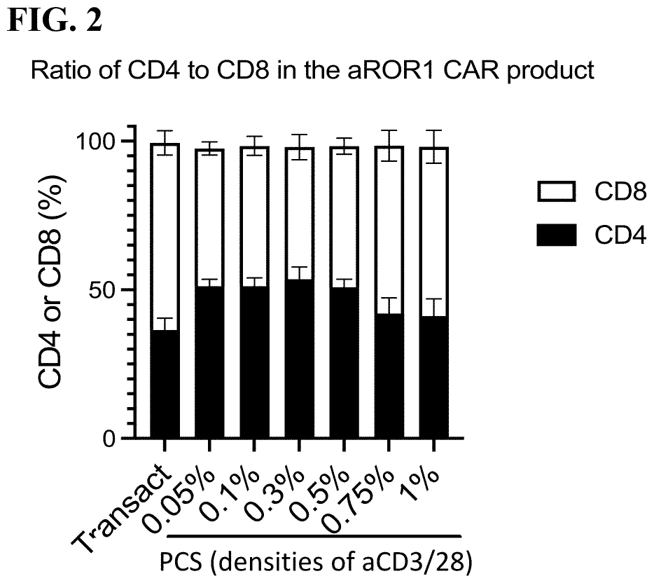

To assess the effect of anti-CD3/CD28 density on PCS on the CD4 to CD8 ratio in anti-ROR1 CAR-T cell products in MRM, healthy donor T cells were activated using either TRANSACT™ or PCS formulations with varying anti-CD3/28 density (0.1%-1%; Table 1). T cells were transduced with the R12 construct (see, e.g., Hudecek, et al., Clin. Cancer Res. 19.12(2013):3153-64) and cultured in MRM for 8 days. On day 8, the T cell products were stained for surface markers related to T cell phenotype and stemness. The ratio of CD4-positive T cells to CD8-positive T cells was determined (FIG. 2). All PCS-activated T cell products showed heavier CD4-bias compared to TRANSACT™-activated T cell product. Within the PCS products, 0.3% a-CD3/28 (PCS-2) showed the highest CD4-bias, which decreased with increasing a-CD3/28 density. At higher densities, at 0.75% and 1%, the CD4-bias was markedly reduced from the lower densities, and was more comparable with the TRANSACT™ product. These data indicated that PCS has the ability to tune CD4 and CD8 ratios in the context of MRM.

### Example 3

To determine if an optimal range of anti-CD3/CD28 densities on PCS products that can enrich “stem-like” T cells in the MRM anti-ROR1 CAR T cell products, healthy donor T cells were activated using either TRANSACT™ or PCS products with varying anti-CD3/28 densities (0.1%-1%; Table 1). T cells were transduced with the R12 construct and cultured in MRM for 8 days. On day 8, the T cell products were stained for surface markers related to T cell phenotype and stemness.

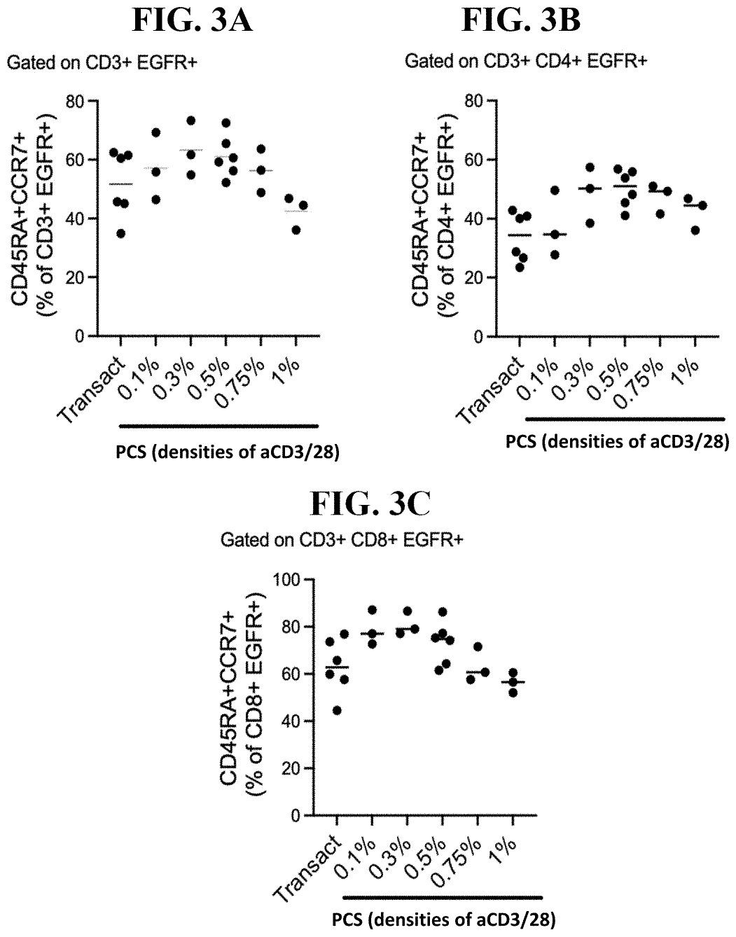

Overall, PCS products at 0.3% and 0.5% densities (PCS-2 and PCS-3, respectively) showed more enrichment in “stem-like” T cells compared to the TRANSACT™ product (FIG. 3A). When looking within the PCS products, 0.3%-0.5% densities (PCS-2 and PCS-3, respectively) showed the highest enrichment of “stem-like” T cells compared to the other densities. These observations were shown in the CD4 (FIG. 3B) and CD8 populations as well (FIG. 3C).

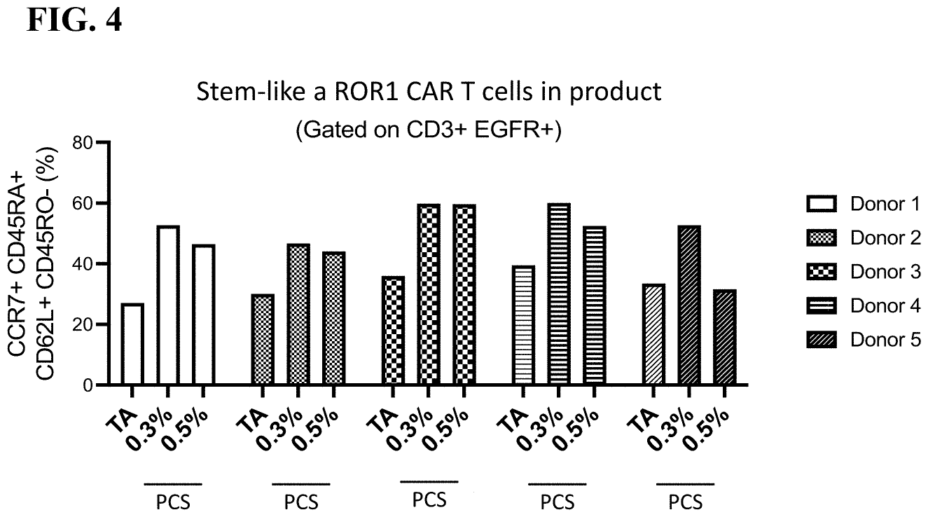

Furthermore, PCS products at 0.3% and 0.5% aCD3/28 densities (PCS-2 and PCS-3, respectively) were evaluated in additional healthy donors (n=5) using a more rigorous surface marker panel to identify “stem-like” T cells in the product. Both PCS products (PCS-2 and PCS-3) showed a stronger enrichment in stem-like T cells compared to the TRANSACT™ product. Specifically, PCS-2 showed stronger enrichment in all 5 donors and PCS-3 showed stronger enrichment in 4 out of 5 donors (FIG. 4). Collectively, these data suggest that PCS can synergize with MRM to enhance T cell sternness.

### Example 4

To further assess the effect that conditions comprising a metabolic reprogramming media in combination with PCS has on T cells, healthy donor T cells were activated using either TRANSACT™ or PCS products at 0.3% or 0.5% a-CD3/28 density (PCS-2 and PCS-3, respectively). T cells were transduced with the R12 construct and cultured in MRM for 7 days. On day 7, the T cell products were cryopreserved. Subsequently, the T cells were thawed and evaluated for their ability to upregulate cytokine expression in response to target stimulation.

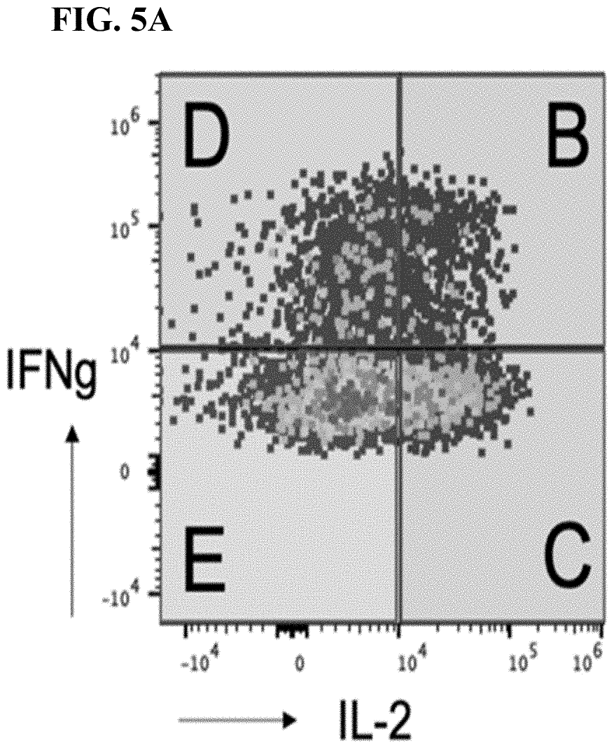

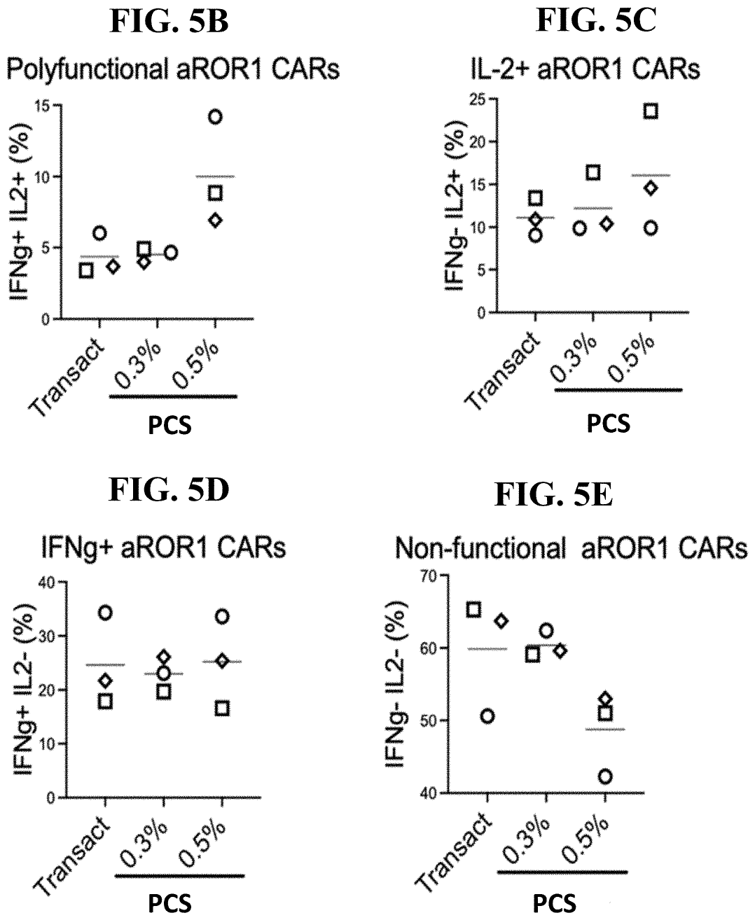

T cell functionality can be described as their ability to express IL-2 and IFNg in response to target stimulation. FIG. 5A shows a representative flow cytometry plot of intracellular IL-2 and IFN-gamma (IFNg) and gating strategy for intracellular cytokine analysis. T cells were first gated on live EGFR+ CD45+ CD3+ T CAR-T cells, and subsequently gated by IFNg and IL-2 expression. The PCS product at 0.5% aCD3/28 (PCS-3) showed better polyfunctionality (IFN+ and IL2+) compared to the TRANSACT™ product in all donors tested (FIG. 5B). The PCS product using PCS-3 also showed higher IL2+ alone (FIG. 5C) and comparable IFNg+ alone (FIG. 5D) T cells compared to the TRANSACT™ product. Consequently, the PCS product using PCS-3 showed the lowest non-functional, or T cells that express neither IL2 nor IFNg, T cells (FIG. 5E). Collectively, these data suggest that PCS can synergize with MRM to generate more polyfunctional CAR T cells.

### Example 5

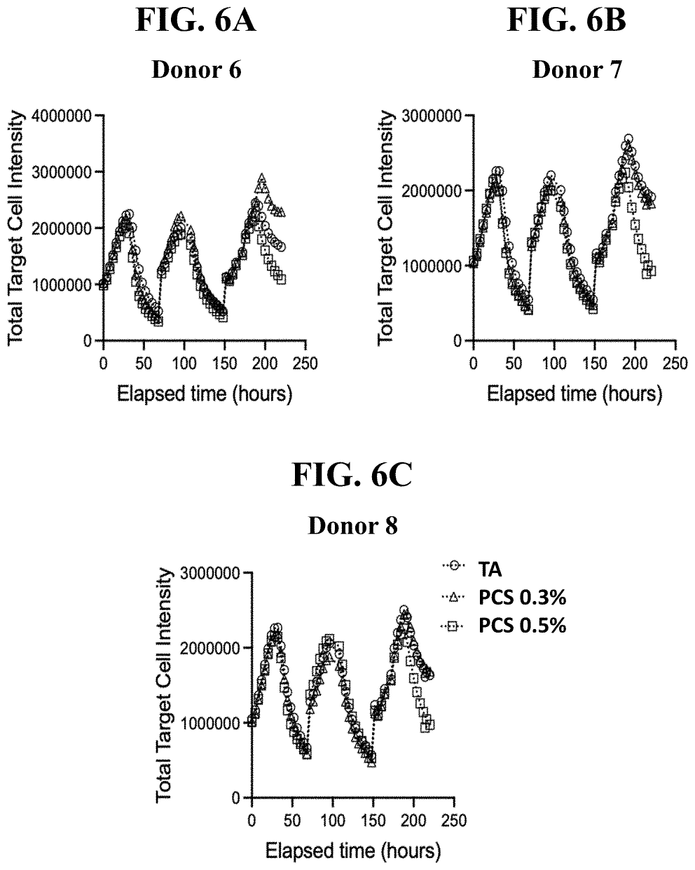

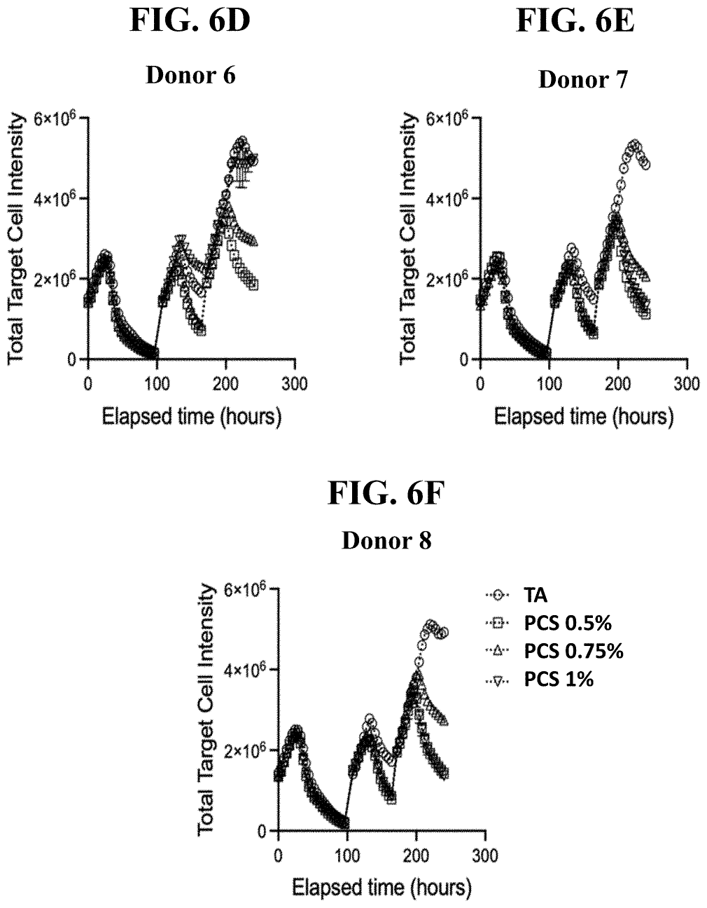

To assess target clearance by PCS produced CAR-T cells in vitro, healthy donor T cells were activated using either TRANSACT™ or PCS products at 0.3% or 0.5% a-CD3/28 density (PCS-2 and PCS-3, respectively) (FIGS. 6A-6C). In a separate study, healthy donor T cells were activated using either TRANSACT™ or PCS products at 0.5%, 0.75% or 1% a-CD3/28 density (PCS-3, PCS-4 and PCS-5, respectively) (FIGS. 6D-6F). T cells were transduced with the R12 construct and cultured in MRM for 7 days. On day 7, the T cell products were cryopreserved. Subsequently, the T cells were thawed and evaluated for their ability to repeatedly kill target cells using a sequential stimulation assay. Briefly, cryopreserved T cells were thawed, rested and resuspended in Full RP10 media. T cells were counted. 4,000 EGFR+ CAR T cells were co-cultured with 20,000 ROR1+targets (H1975) at 1:5 E:T in flat bottom 96-well plates. Every 3-4 days, 25% of the previous culture was transferred into a new plate with fresh targets plated at the initial seeding density. Separately, every 3-4 days, the number of EGFR+ CAR T cells was determined using flow cytometry. Target clearance was quantified using INCUCYTE®.

In both studies, PCS at 0.5% aCD3/28 density (PCS-3) showed better persistent target clearance compared to the TRANSACT™ product in 3/3 donors (FIGS. 6A-6F). PCS at 0.5% aCD3/28 density showed better target clearance than the 0.3% aCD3/28 density, which was comparable to the TRANSACT™ product (FIGS. 6A-6C). PCS at 0.5% aCD3/28 density also showed better target clearance than the 0.75% and 1% formulations, which also both outperformed the TRANSACT™ product (FIGS. 6D-6F). Collectively, these studies suggest that PCS can synergize with MRM to generate highly functionally potent CAR-T cells.

### Example 6

To further validate that PCS products show superior potency for CAR-T cell mediated target clearance, healthy donor T cells were activated using either TRANSACT™ or PCS at 0.5% a-CD3/28 density (PCS-3). T cells were transduced with the R12 construct and cultured in MRM for 7 days. On day 7, the T cell products were.

In Example 5, PCS products showed superior repeated target clearance as compared to the TRANSACT™ product. Subsequently, CAR-T cells were stress-tested to assess their short-term potency. To do this, the T cells were thawed and co-cultured with targets at low effector to target ratios (E:T) as described briefly as follows: cryopreserved T cells were thawed, rested and resuspended in Full RP10 media. T cells were counted. 20,000 per well NLR+ ROR1+ target cells (NLR+H1975) were plated in a flat-bottom 96 well plate and allowed to adhere for 2 hours prior to adding T cells. EGFR+ CAR T cells were then added to the NLR+H1975 targets at 1:125 (1 T cell for 125 target cell, E:T) in total 200 ul media and transferred to the INCUCYTE®. At least 2 replicates were used. Target clearance was quantified using INCUCYTE® over 4 days

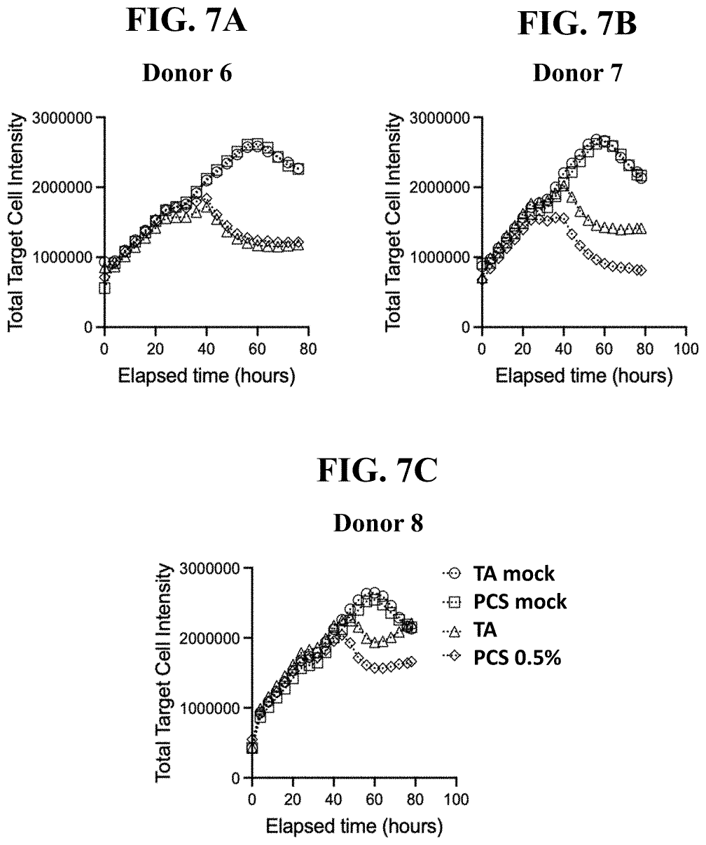

Consistent with what was demonstrated in Example 5, the PCS CAR-T product showed superior short-term potency compared to the TRANSACT™ product in 2/3 donors (FIGS. 7A-7B). In the other donor, the PCS product performed similarly to the TRANSACT™ process (FIG. 7C).

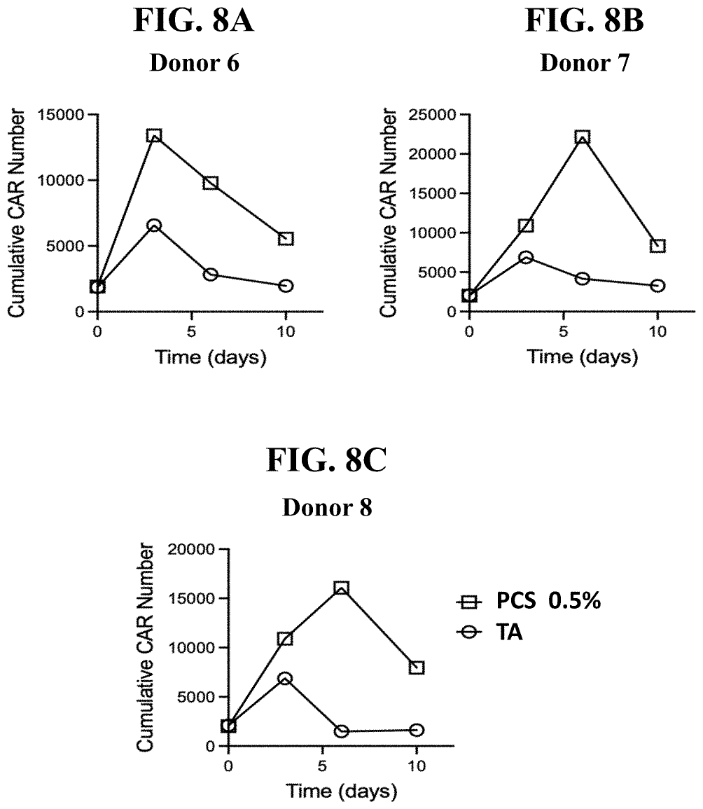

Consistent with the observation that PCS products showed superior target clearance in the sequential stimulation assay, PCS T cells also showed more rapid expansion over time compared to the TransAct product in 3/3 donors (FIGS. 8A-8C).

### Example 7

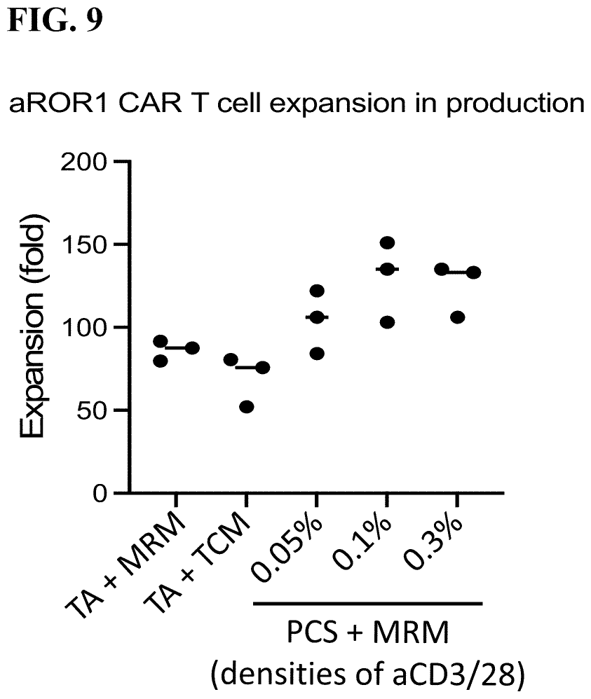

To determine if PCS+MRM cultured T cells exhibit superior expansion compared to either TRANSACT™+MRM or TRANSACT™+TCM, healthy donor T cells were activated using either TRANSACT™ or PCS formulations with varying anti-CD3/28 density (0.1%-0.3%) in MRM. T cells were transduced with the R12 construct and cultured in TCM (control T cell media comprising about 5 mM potassium ion) or MRM for 8 days. On day 8, the T cells were counted and the total expansion from day 0 was determined (FIG. 9). T cells produced in TRANSACT™+MRM showed modestly reduced expansion compared to TRANSACT™+TCM. On the other hand, T cells produced in PCS+MRM showed higher expansion compared to the TRANSACT™-activated T cell product in TCM or MRM.

### Example 8

To determine if PCS+MRM cultured T cells exhibit an increase in the “stem-like” population in the anti-ROR1 CAR T cell product compared to either TRANSACT™+MRM or TRANSACT™+TCM, healthy donor T cells were activated and cultured using TRANSACT™ in TCM, TRANSACT™ in MRM, or PCS formulations with varying anti-CD3/28 density in MRM. T cells were transduced with the R12 construct and cultured in TCM or MRM for 8 days. On day 8, the T cell products were stained for surface markers related to T cell phenotype and stemness as described in Example 3.

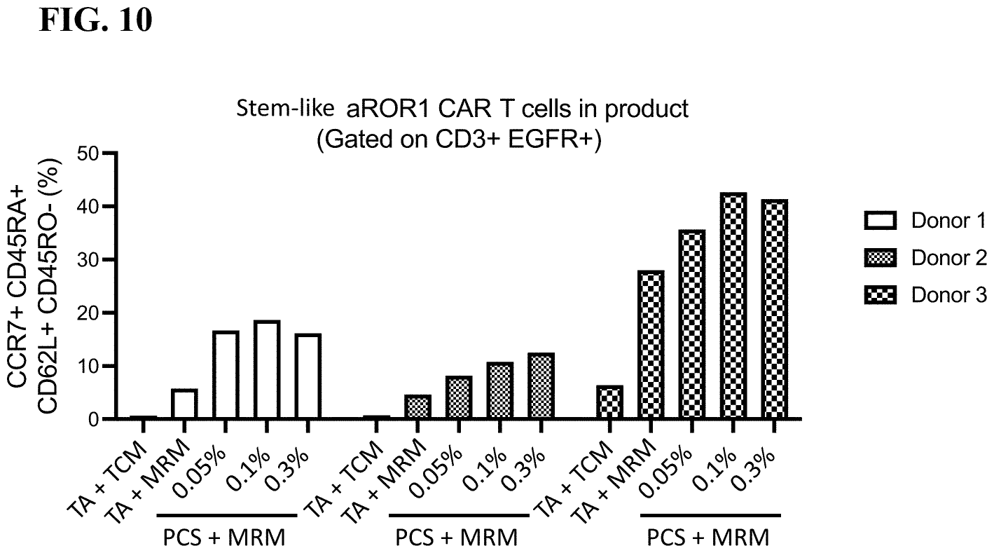

In 3 out of 3 donors tested, the TRANSACT™+MRM product showed a higher “stem-like” T cell population compared to TRANSACT™+TCM. PCS+MRM products further showed an enrichment in the “stem-like” T cell population over the TRANSACT™+MRM product (FIG. 10). These data suggest that PCS+MRM shows at least an additive benefit over the TRANSACT™ and MRM controls.

### Example 9

To assess whether PCS+MRM shows an enhancement of polyfunctional CAR-T cells in response to target stimulation, healthy donor T cells were activated and cultured using TRANSACT™ in TCM, TRANSACT™ in MRM, or PCS at 0.5% anti-CD3/28 density in MRM (PCS-3) at the 1M scale. T cells were transduced with the R12 construct and cultured in either TCM or MRM for 7 days. On day 7, the T cell products were cryopreserved. Subsequently, the T cells were thawed and evaluated for their ability to upregulate cytokine expression in response to target stimulation.

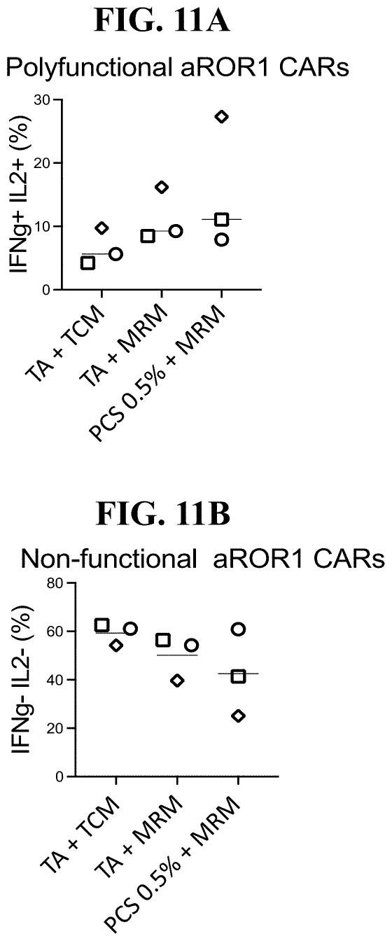

T cell functionality can be described as their ability to express IL-2 and IFNg in response to target stimulation (FIG. 5A) and T cell polyfunctionality has been implicated in positive correlation with clinical outcomes. The TRANSACT™+MRM product showed higher polyfunctionality (IFN+ and IL-2+) than the TRANSACT™+TCM product. Furthermore, the PCS+MRM product showed higher polyfunctionality than the TRANSACT™+MRM product (FIG. 11A). Conversely, the PCS+MRM product showed the least “non-functional” T cells (IFN- and IL-2) in a stepwise manner compared to the TRANSACT™+MRM product and the TRANSACT™+TCM product (FIG. 11B).

### Example 10

To assess whether PCS+MRM shows an enhancement in persistent target clearance by CAR-T cells in vitro, healthy donor T cells were activated and cultured using TRANSACT™ in TCM, TRANSACT™ in MRM, or PCS at 0.5% anti-CD3/28 density in MRM (PCS-3) at the 1M scale. T cells were transduced with the R12 construct and cultured in either TCM or MRM for 7 days. On day 7, the T cell products were cryopreserved. Subsequently, T cells were thawed and evaluated for their ability to repeatedly kill target cells using a sequential stimulation and a serial stimulation assay. The goal of the sequential stimulation assay is to assess the functional potency of the T cell population as a whole, whereas the goal of the serial stimulation assay is to assess the potency of individual T cells over time. The sequential stimulation assay was performed as follows. Cryopreserved T cells were thawed, rested and resuspended in Full RP10 media. T cells were counted. In the serial-stimulation plate, EGFR+ CAR T cells were co-cultured with ROR1+targets (H1975) at the effector:target ratio of 1:5. The number of cells was determined using the size of the culture vessel. Separately, in the INCUCYTE® plate used to visualize and quantify target clearance, 4,000 EGFR+ CAR T cells were co-cultured with 20,000 ROR1+targets (H1975) at 1:5 E:T in flat bottom 96-well plates. Every 3-4 days, the number of EGFR+ CAR T cells in the serial-stimulation plate was determined using flow cytometry. Fresh target cells were again plated with EGFR+ CAR T cells at the effector:target ratio of 1:5 (resetting the E:T) in the serial-stimulation plate. Separately, 4,000 serially-stimulated CAR T cells were co-cultured with 20,000 ROR1+targets (H1975) at 1:5 E:T in flat bottom 96-well plates for INCUCYTE® analysis. Target clearance was quantified using INCUCYTE®.

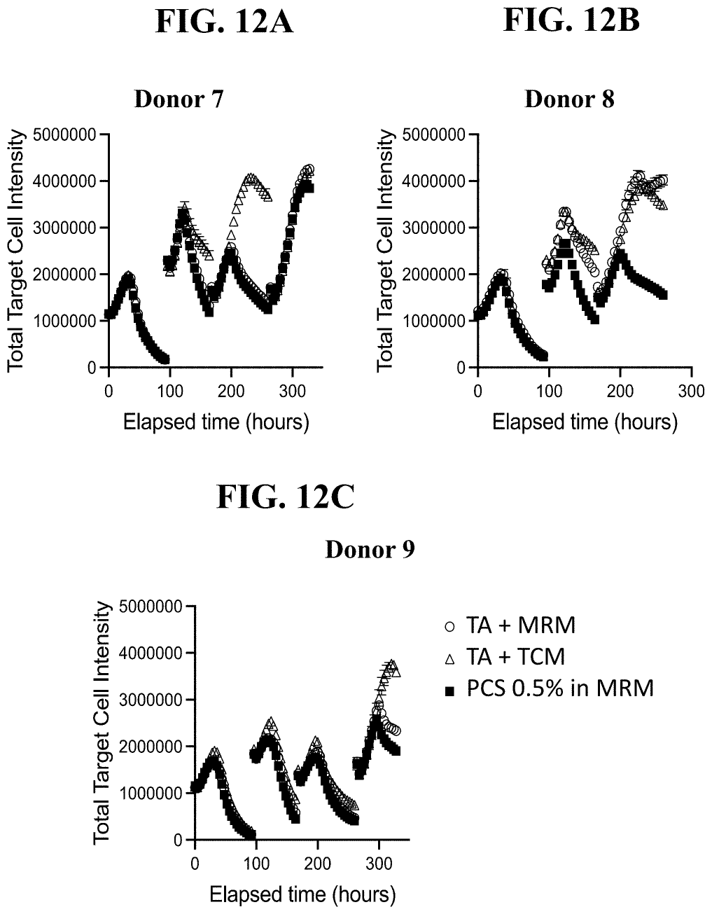

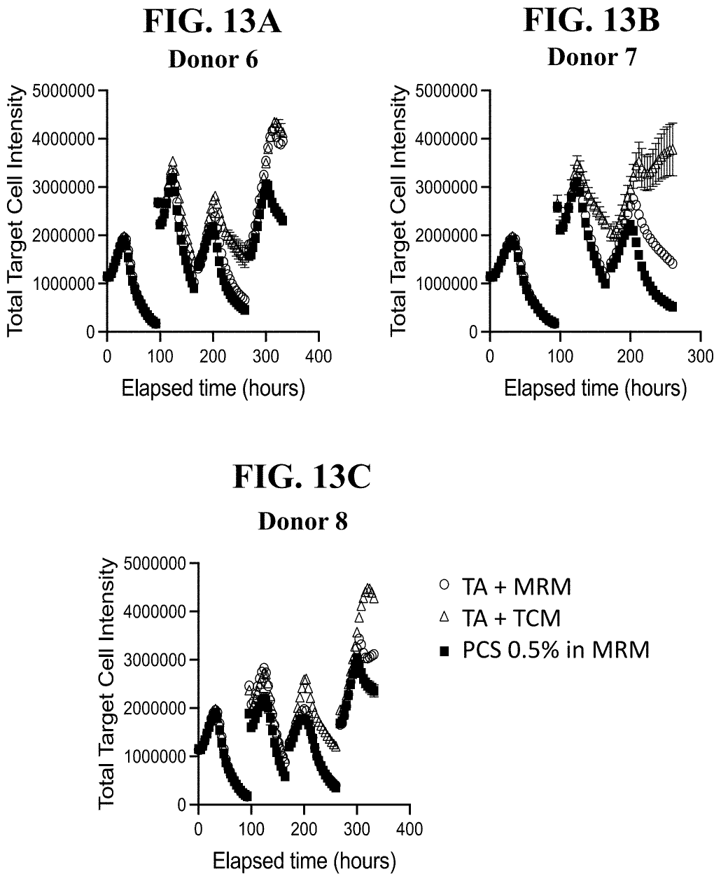

In 3 out of 3 donors tested, the TRANSACT™+MRM product outperformed the TRANSACT™+TCM product, in both sequential and serial stimulation settings (FIGS. 12A-13C). The PCS+MRM product further showed superior target clearance over TRANSACT™+MRM in the sequential stimulation assay in 2 out of 3 donors (FIGS. 12B-12C), with the last donor showing comparable effects with TRANSACT™+MRM (FIG. 12A). In the serial stimulation assay, the PCS+MRM product outperformed the TRANSACT™+MRM product in 3 out of 3 donors (FIGS. 13A-13C).

### Example 11

To evaluate PCS in MRM variants, two PCS formulations (0.1% density and 0.5% density), five MRM variants (MRM-1, MRM-2, MRM-3, MRM-4, and standard MRM), and 1 TCM media were used. T cells were stimulated with either PCS formulation, transduced with R12, and cultured in TCM, MRM-1 to MRM-4, or standard MRM. MRM variants differed from each other only by the concentrations of K⁺ ion and NaCl. K ion levels decreased from MRM-1 (highest K⁺ ion level) to MRM-4 (lowest K⁺ ion levels), while the concentration of NaCl increased from MRM-1 (lowest NaCl concentration) to MRM-4 (highest NaCl concentration). T cells were analyzed for activation following stimulation, and expansion and transduction efficiency following 7 days in culture.

Healthy human donor CD4 and CD8 T cells were thawed, washed once and resuspended in pre-warmed MRM. CD4 and CD8 T cells were then mixed at a 1:1 ratio and then spun down and resuspended to 2e6 total T cells/ml in the respective media.

Pre-lyophilized PCS materials of two different PCS formulations were resuspended in Complete TCM to 10 mg/ml. Subsequently, 1e6 T cells at 1:1 CD4:CD8 were activated by mixing with 20 ul of PCS. Finally, T cells were transduced with the R12 construct at MOI 7.5 and left undisturbed for 72 hours.

T cells were plated in complete TCM, MRM-1, MRM-2, MRM-3, MRM-4, or standard MRM, all supplemented with IL2/7/15, stimulated using either PCS formulation, and transduced on Day 0. On day 2, T cells were transferred into GRex24 in 5 ml of complete TCM, MRM-1, MRM-2, MRM-3, MRM-4, or standard MRM, all supplemented with IL2/7/15. On day 5, T cells were transferred into GRex6 in total of 15 or 20 ml of complete TCM, MRM-1, MRM-2, MRM-3, MRM-4, or standard MRM, all supplemented with IL2/7/15. T cells were collected for phenotype analysis on day 2 and day 7.

At the end of T cell production, T cells were counted. 500,000 T cells were collected for flow cytometry. T cells were stained with surface antibodies for 20 minutes at 4 C. Cells were washed and visualized on the BioRad ZE5 or the Cytek Aurora flow cytometer.

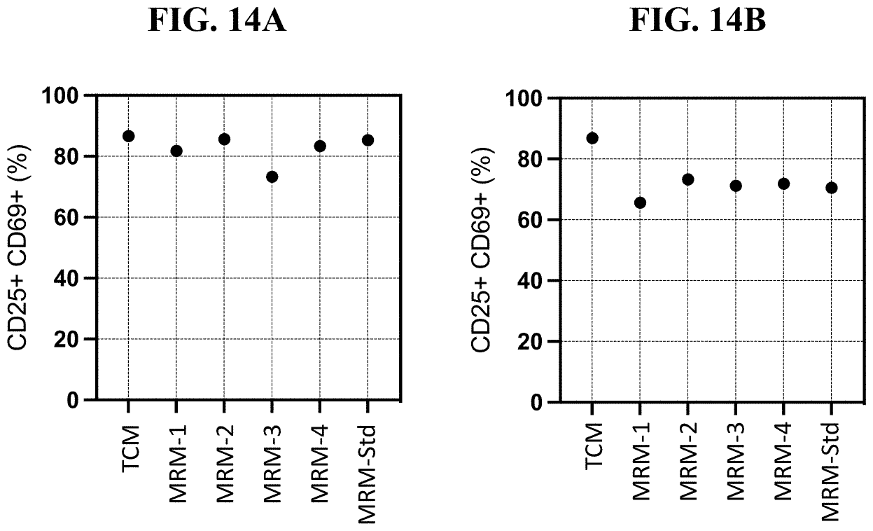

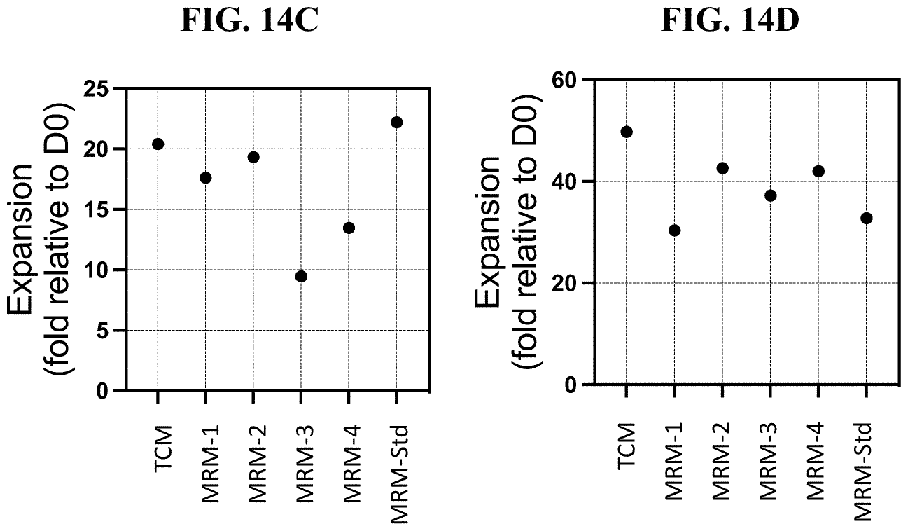

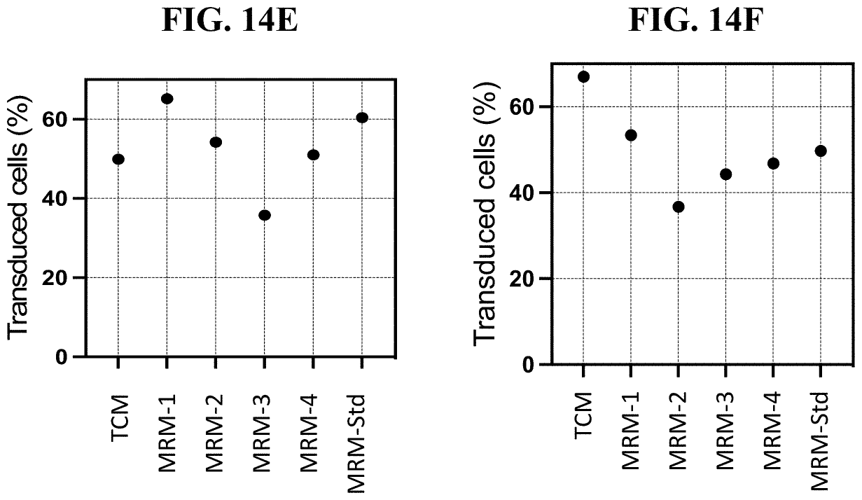

T cells stimulated with either PCS formulation showed successful activation, expansion, and transduction in all media formulations. At day 2 after T cell stimulation with either PCS formulation, T cells cultured in the various MRM formulations showed similar expression of the activation markers CD25 and CD69, and all media formulations in both PCS formulations resulted in successful T cell activation (FIGS. 14A-14B).

At 7 days following T cell stimulation with either PCS formulation, cells were counted on the Cellaca MX High-Throughput automatic counter. Expansion was calculated as the total number of viable cells on day 7 divided by the total number of viable cells on day 0. All media formulations in both PCS formulations resulted in successful T cell expansion (FIGS. 14C-14D).

At 7 days following T cell stimulation with either PCS formulation, cells were collected and evaluated for transduction efficiency. Dead cells were excluded from the analysis using the viability dye. Transduced T cells were defined as T cells that are CD3+EGFR+. All media formulations in both PCS formulations resulted in successful T cell transduction (FIGS. 14E-14F).

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The contents of all cited references (including literature references, U.S. or foreign patents or patent applications, and websites) that are cited throughout this application are hereby expressly incorporated by reference as if written herein in their entireties for any purpose, as are the references cited therein. Where any inconsistencies arise, material literally disclosed herein controls.

1. A method of preparing a population of human immune cells for immunotherapy comprising contacting human immune cells with a programmable cell-signaling scaffold (PCS) in a medium comprising potassium ion at a concentration higher than 5 mM.

2. A method of activating a population of human immune cells for immunotherapy comprising contacting human immune cells with a programmable cell-signaling scaffold (PCS) in a medium comprising potassium ion at a concentration higher than 5 mM.

3. A method of increasing the yield and/or stemness of activated human immune cells during ex vivo or in vitro culture comprising contacting human immune cells with a programmable cell-signaling scaffold (PCS) in a medium comprising potassium ion at a concentration higher than 5 mM.

4-5. (canceled)

6. The method of claim 1 5, wherein the PCS comprises (i) high surface area mesoporous silica micro-rods (MSR); (ii) a fluid-supported lipid bilayer (SLB) layered on the MSR base layer; (iii) a plurality of surface cues presented on the SLB; and (iv) a plurality of soluble cues presented on the MSR.

7-8. (canceled)

9. The method of claim 6, wherein the soluble cue is released from the scaffold in a controlled-release manner.

10. (canceled)

11. The method of claim 6, wherein the plurality of soluble cues comprises IL-1, IL-2, IL-4, IL-5, IL-7, IL-10, IL-12, IL-15, IL-17, IL-21, transforming growth factor beta (TGF-β), or an agonist thereof, a mimetic thereof, a variant thereof, a functional fragment thereof, or a combination thereof.

12. The method of claim 6, wherein the plurality of soluble cues comprises:
(a) (i) IL-2, an agonist thereof, a mimetic thereof, a variant thereof, a functional fragment thereof, or a combination thereof and (ii) a second soluble cue comprising IL-7, IL-21, IL-15, IL-15 superagonist, or any combination thereof;
(b) (i) IL-2, an agonist thereof, a mimetic thereof, a variant thereof, a functional fragment thereof, or a combination thereof, (ii) a second soluble cue comprising IL-7, IL-21, IL-15, IL-15 superagonist, or any combination thereof, and (iii) a third soluble cue comprising IL-7, IL-21, IL-15, IL-15 superagonist, or any combination thereof;
(c) an N-terminal IL-2 fragment comprising the first 30 amino acids of IL-2 (pl-30), an IL-2 superkine peptide, an IL-2 partial agonist peptide, or a combination thereof;
(d) a T-cell stimulatory molecule, a T-cell co-stimulatory molecule, or both a T-cell stimulatory molecule and a T cell co-stimulatory molecule; or
(e) any combination of (a)-(d).

13-15. (canceled)

16. The method of claim 12, wherein the T-cell stimulatory molecule and the T-cell co-stimulatory molecule are each, independently,
(a) (i) loaded, (ii) coated, and/or (iii) partly embedded onto the lipid bilayer (SLB);
(b) presented on the mesoporous silica micro-rods (MSR); or
(c) both (a) and (b).

17-23. (canceled)

24. The method of claim 12, wherein the T-cell stimulatory molecule comprises:
(a) an anti-CD3 antibody or an antigen-binding portion thereof, an anti-macrophage scavenger receptor (MSR1) antibody or an antigen-binding portion thereof, an anti-T-cell receptor (TCR) antibody or an antigen-binding portion thereof, an anti-CD2 antibody or an antigen-binding portion thereof, an anti-CD47 antibody or an antigen-binding portion thereof, a major histocompatibility complex (MHC) molecule loaded with an MHC peptide or a multimer thereof, an MHC-immunoglobulin (Ig) conjugate or a multimer thereof, or a combination thereof;
(b) an antibody, or an antigen-binding portion thereof, which specifically binds to a co-stimulatory antigen comprising CD28, 4.1BB (CD137), OX40 (CD134), CD27 (TNFRSF7), GITR (CD357), CD30 (TNFRSF8), HVEM (CD270), LTfiR (TNFRSF3), DR3 (TNFRSF25), ICOS (CD278), CD226 (DNAM1), CRTAM (CD355), TIM1 (HAVCR1, KIM1), CD2 (LFA2, 0X34), SLAM (CD150, SLAMF1), 2B4 (CD244, SLAMF4), Ly108 (NTBA, CD352, SLAMF6), CD84 (SLAMF5), Ly9 (CD229, SLAMF3), CRACC (CD319, BLAME), or any combination thereof; or
(c) any combination of (a) and (b).

25. (canceled)

26. The method of claim 12, wherein the T-cell stimulatory molecule and the T-cell co-stimulatory molecule comprise
(a) bispecific antibodies or antigen binding portions thereof;
(b) a pair comprising CD3/CD28, CD3/ICOS, CD3/CD27, CD3/CD137, or a combination thereof; or
(c) any combination of (a) and (b).

27. (canceled)

28. The method of claim 6, wherein the scaffold further comprises
(a) an immunoglobulin molecule that binds specifically to an Fc-fusion protein;
(b) a recruitment compound comprising granulocyte macrophage-colony stimulating factor (GM-CSF), chemokine (C—C motif) ligand 21 (CCL-21), chemokine (C—C motif) ligand 19 (CCL-19), Chemokine (C—X—C Motif) ligand 12 (CXCL12), interferon gamma (IFNy), a FMS-like tyrosine kinase 3 (Flt-3) ligand, or any combination thereof;
(c) an antigen;
(d) any combination of (a) to (c).

29-38. (canceled)

39. The method of claim 1, wherein the immune cells comprise a polynucleotide encoding an antigen receptor selected from an antibody, an engineered antibody such as scFv, a CAR, an engineered TCR, a TCR mimic, a chimeric signaling receptor (CSR), or any combination thereof.

40-41. (canceled)

42. The method of claim 39, wherein the antigen receptor comprises:
(a) (i) an antigen-binding domain, (ii) a transmembrane domain, (iii) a costimulatory domain, (iv) an intracellular signaling domain, or (v) any combination of (i)-(iv);
(b) an engineered TCR.

43. The method of claim 42, wherein
(a) the antigen-binding domain specifically binds an antigen selected from the group consisting of AFP (alpha-fetoprotein), αvβ6 or another integrin, BCMA, Braf, B7-H3, B7-H6, CA9 (carbonic anhydrase 9), CCL-1 (C—C motif chemokine ligand 1), CD5, CD19, CD20, CD21, CD22, CD23, CD24, CD30, CD33, CD38, CD40, CD44, CD44v6, CD44v7/8, CD45, CD47, CD56, CD66e, CD70, CD74, CD79a, CD79b, CD98, CD123, CD138, CD171, CD352, CEA (carcinoembryonic antigen), Claudin 18.2, Claudin 6, c-MET, DLL3 (delta-like protein 3), DLL4, ENPP3 (ectonucleotide pyrophosphatase/phosphodiesterase family member 3), EpCAM, EPG-2 (epithelial glycoprotein 2), EPG-40, ephrinB2, EPHa2 (ephrine receptor A2), ERBB dimers, estrogen receptor, ETBR (endothelin B receptor), FAP-α (fibroblast activation protein α), fetal AchR (fetal acetylcholine receptor), FBP (a folate binding protein), FCRL5, FR-α (folate receptor alpha), GCC (guanyl cyclase C), GD2, GD3, GPC2 (glypican-2), GPC3, gp100 (glycoprotein 100), GPNMB (glycoprotein NMB), GPRC5D (G Protein Coupled Receptor 5D), HER2, HER3, HER4, hepatitis B surface antigen, HLA-A1 (human leukocyte antigen A1), HLA-A2 (human leukocyte antigen A2), HMW-MAA (human high molecular weight-melanoma-associated antigen), IGF1R (insulin-like growth factor 1 receptor), Ig kappa, Ig lambda, IL-22Ra (IL-22 receptor alpha), IL-13Ra2 (IL-13 receptor alpha 2), KDR (kinase insert domain receptor), LI cell adhesion molecule (LI-CAM), Liv-1, LRRC8A (leucine rich repeat containing 8 Family member A), Lewis Y, melanoma-associated antigen (MAGE)-A1, MAGE-A3, MAGE-A6, MART-1 (melan A), murine cytomegalovirus (MCMV), MCSP (melanoma-associated chondroitin sulfate proteoglycan), mesothelin, mucin 1 (MUC1), MUC16, MHC/peptide complexes (e.g., HLA-A complexed with peptides derived from AFP, KRAS, NY-ESO, MAGE-A, and WT1), NCAM (neural cell adhesion molecule), Nectin-4, NKG2D (natural killer group 2 member D) ligands, NY-ESO, oncofetal antigen, PD-1, PD-L1, PRAME (preferentially expressed antigen of melanoma), progesterone receptor, PSA (prostate specific antigen), PSCA (prostate stem cell antigen), PSMA (prostate specific membrane antigen), ROR1, ROR2, SIRPα (signal-regulatory protein alpha), SLIT, SLITRK6 (NTRK-like protein 6), STEAP1 (six transmembrane epithelial antigen of the prostate 1), survivin, TAG72 (tumor-associated glycoprotein 72), TPBG (trophoblast glycoprotein), Trop-2, VEGFR1 (vascular endothelial growth factor receptor 1), VEGFR2, and antigens from HIV, HBV, HCV, HPV, and other pathogens, and any combination thereof; or
(b) the engineered TCR specifically binds a tumor antigen/MHC complex derived from AFP, CD19, BCMA, CLL-1, CS1, CD38, CD19, TSHR, CD123, CD22, CD30, CD171, CD33, EGFRvIII, GD2, GD3, Tn Ag, PSMA, ROR1, ROR2, GPC1, GPC2, FLT3, FAP, TAG72, CD44v6, CEA, EPCAM, B7H3, KIT, IL-13Ra2, mesothelin, IL-1 1Ra, PSCA, PRSS21, VEGFR2, LewisY, CD24, PDGFR-beta, SSEA-4, CD20, folate receptor alpha, ERBB2 (Her2/neu), MUC1, MUC16, EGFR, NCAM, prostase, PAP, ELF2M, Ephrin B2, IGF-I receptor, CAIX, LMP2, gplOO, bcr-abl, tyrosinase, EphA2, fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, surviving, telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, CD2, CD3ε, CD4, CD5, CD7, the extracellular portion of the APRIL protein, neoantigen, or any combinations thereof.

44-64. (canceled)

65. The method of claim 1, wherein the concentration of potassium ion is about 50 mM, about 60 mM, or about 70 mM.

66. (canceled)

67. The method of claim 1, wherein the medium further comprises
(a) NaCl; wherein the sum of the potassium ion concentration and the NaCl concentration, multiplied by two is (i) more than 240 and less than 280 or (ii) is more than or equal to 280 and less than 300;
(b) one or more cytokines, optionally selected from the group consisting of interleukin-2 (IL-2), interleukin-7 (IL-7), interleukin-21 (IL-21), interleukin-15 (IL-15), or any combination thereof;
(c) calcium ion;
(d) glucose;
(e) a cell expansion agent, optionally selected from the group consisting of a GSK3B inhibitor, an ACLY inhibitor, a PI3K inhibitor, an AKT inhibitor, or any combination thereof; or
(f) any combination of (a)-(e).

68-109. (canceled)

110. A population of human immune cells prepared by the method of claim 1.

111-116. (canceled)

117. A pharmaceutical composition comprising the population of human immune cells of claim 110, and a pharmaceutically acceptable carrier.

118. A method of killing target cells, comprising contacting the target cells with the population of immune cells of claim 110 under conditions that allow killing of the target cells by the immune cells.

119. A method of treating a patient in need thereof, comprising administering the population of human immune cells of claim 110 to the patient.
